(19) United States
(12) Patent Application Publication
(10) Pub. No.: US 2025/0051339 A1
(43) Pub. Date: Feb. 13, 2025

(54) METHODS FOR THE SYNTHESIS OF COMPLEMENT FACTOR D INHIBITORS

(71) Applicant: Alexion Pharmaceuticals, Inc., Boston, MA (US)

(72) Inventor: Akihiro Hashimoto, Guilford, CT (US)

(73) Assignee: Alexion Pharmaceuticals, Inc., Boston, MA (US)

(21) Appl. No.: 18/719,261

(22) PCT Filed: Dec. 13, 2022
(86) PCT No.: PCT/US2022/052690

§ 371 Date: Jun. 13, 2024

Related U.S. Application Data
(60) Provisional application No. 63/289,736, filed on Dec. 15, 2021.

(51) Int. Cl. C07D 487/04; C07D 403/04

(52) U.S. Cl. CPC C07D 487/04; C07D 403/04

(57) ABSTRACT

The present disclosure provides methods for the synthesis of complement factor D inhibitors and intermediates thereof. The methods involve subjecting an intermediate in the synthesis of a complement factor D inhibitor to a t-butyl ester deprotection reaction using a weak base.

### BACKGROUND

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. Complement Factor D plays an early and central role in activation of the alternative pathway of the complement cascade. Activation of the alternative complement pathway is initiated by spontaneous hydrolysis of a thioester bond within the C3 protein to produce C3(H₂O), which associates with Factor B to form the C3(H₂O)B complex. Complement Factor D acts to cleave Factor B within the C3(H₂O)B complex to form Ba and Bb. The Bb fragment remains associated with C3(H₂O) to form the alternative pathway C3 convertase C3(H₂O)Bb. Additionally, C3b generated by any of the C3 convertases also associates with Factor B to form C3bB, which Factor D cleaves to generate the later stage alternative pathway C3 convertase C3bBb. This latter form of the alternative pathway C3 convertase may provide important downstream amplification within all three of the defined complement pathways, leading ultimately to the recruitment and assembly of additional factors in the complement cascade pathway, including the cleavage of C5 to C5a and C5b. C5b acts in the assembly of factors C6, C7, C8, and C9 into the membrane attack complex, which can destroy pathogenic cells by lysing the cell.

The dysfunction of or excessive activation of complement has been linked to certain autoimmune, inflammatory, and neurodegenerative diseases, as well as ischemia-reperfusion injury and cancer. For example, activation of the alternative pathway of the complement cascade contributes to the production of C3a and C5a, both potent anaphylatoxins, which also have roles in a number of inflammatory disorders. Therefore, in some instances, it is desirable to decrease the response of the complement pathway, including the alternative complement pathway. Some examples of disorders mediated by the complement pathway include age-related macular degeneration (AMD), paroxysmal nocturnal hemoglobinuria (PNH), multiple sclerosis, and rheumatoid arthritis.

Additional complement-mediated disorders include those classified under component 3 glomerulopathy (C3G). C3G is a recently defined entity comprised of dense deposit disease (DDD) and C3 glomerulonephritis (C3GN) which encompasses a population of chronic kidney diseases wherein elevated activity of the alternative complement pathway and terminal complement pathway results in glomerular deposits made solely of complement C3 and no immunoglobulin (Ig).

Immune-complex membranoproliferative glomerulonephritis (IC-MPGN) is a renal disease which shares many clinical, pathologic, genetic and laboratory features with C3G, and therefore can be considered a sister disease of C3G. In the majority of patients with IC-MPGN, an underlying disease or disorder—most commonly infections, autoimmune diseases, or monoclonal gammopathies—are identified to which the renal disease is secondary. Patients with idiopathic IC-MPGN can have low C3 and normal C4 levels, similar to those observed in C3G, as well as many of the same genetic or acquired factors that are associated with abnormal alternative pathway activity. Although there are current hypotheses suggesting that the majority of IC-MPGN is attributable to over activity of the classical pathway, those patients with a low C3 and a normal C4 are likely to have significant overactivity of the alternative pathway. IC-MPGN patients with a low C3 and a normal C4 may benefit from alternative pathway inhibition.

Other disorders that have been linked to the complement cascade include atypical hemolytic uremic syndrome (aHUS), hemolytic uremic syndrome (HUS), abdominal aortic aneurysm, hemodialysis complications, hemolytic anemia, or hemodialysis, neuromyelitis optica (NMO), myasthenia gravis (MG), fatty liver, nonalcoholic steatohepatitis (NASH), liver inflammation, cirrhosis, liver failure, dermatomyositis, and amyotrophic lateral sclerosis.

Factor D is an attractive target for inhibition or regulation of the complement cascade due to its early and essential role in the alternative complement pathway, and for its potential role in signal amplification within the classical and lectin complement pathways. Inhibition of Factor D effectively interrupts the pathway and attenuates the formation of the membrane attack complex.

To this end, a number of small molecule Factor D inhibitors have been developed and investigated for potential therapeutic uses. Examples of these Factor D inhibiting compounds methods of preparing them are described in PCT patent publications WO2015/130838, WO2017/035353, WO2017/035409, WO2018/160891, and WO2018/160892.

New methods for the synthesis of small molecule Factor D inhibitors and intermediates thereof are desirable.

### SUMMARY OF THE DISCLOSURE

The present disclosure generally relates to an improved method of preparing compounds useful for treating disorders mediated by complement factor D and intermediates thereof.

Provided herein is a method of preparing a compound of formula (I), which includes reacting a compound of formula (II) with a weak base in a water-miscible organic solvent:

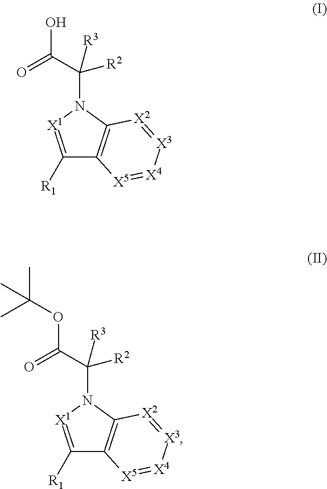

in which R¹ is H; halo; OH; NH₂; cyano; optionally substituted C₁-C₆ alkyl; optionally substituted C₂-C₆ alkenyl; optionally substituted 3- to 8-membered heterocyclyl; —C(O)NRₐRₐ′, wherein each of Rₐ and Rₐ′ is, independently, H, optionally substituted C₁-C₆ alkyl, optionally substituted C₂-C₆ alkenyl, optionally substituted C₂-C₆ alkynyl, or optionally substituted C₃-C₈ cycloalkyl; —C(O)R_{b}; —OC(O)R_{b}; or —C(O)OR_{b}; wherein R_{b}, in each instance, is selected from H, optionally substituted C₁-C₆ alkyl, optionally substituted C₁-C₆ alkoxy, and optionally substituted C₃-C₈ carbocyclyl; each of R² and R³ is, independently, H or optionally substituted C₁-C₆ alkyl; X¹ is N or CR_{c}, wherein R_{c} is H, halo, optionally substituted C₁-C₆ alkyl, or optionally substituted C₁-C₆ alkoxy; each of X² and X⁵ is independently N or CR_{d}, wherein each R_{d} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and each of X³ and X⁴ is independently selected from N, CRₑ, and CR_{f}, wherein Rₑ is selected from H, halo, cyano, optionally substituted C₁-C₆ alkyl, optionally substituted C₁-C₆ alkoxy, and —C(O)OR_{g}, wherein R_{g} is H or optionally substituted C₁-C₆ alkyl; and R_{f} is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S; wherein at least one of X³ and X⁴ is CR_{f}.

In some embodiments, the method further includes preparing a compound of formula (III):

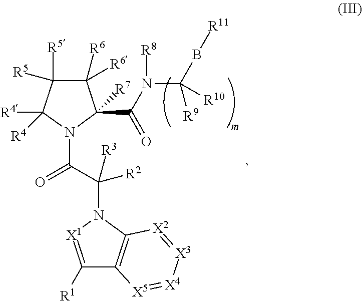

or a pharmaceutically acceptable salt thereof from the compound of formula (I), in which each of R⁴, R^{4′}, R⁵, R^{5′}, R⁶, R^{6′}, and R⁷ is, independently, H; cyano; halo; OH; nitro; optionally substituted C₁-C₆ alkyl; optionally substituted C₂-C₆ alkenyl; C₁-C₆ alkoxy; C₁-C₆ thioalkyl; optionally substituted C₃-C₈ carbocyclyl; optionally substituted C₃-C₈ carbocyclyloxy; —NR_{g}R_{g}′; —C(O)NR_{g}R_{g}′; —OC(O)NR_{g}R_{g}′; —NR_{g}C(O)Rₕ; —NR_{g}C(O)ORₕ; —C(O)Rₕ; —C(O)ORₕ; or —C═NRₕ, wherein each of R_{g}, R_{g}′, and R^{h}, in each instance, is independently selected from H, optionally substituted C₁-C₆ alkyl, optionally substituted C₂-C₆ alkenyl, optionally substituted C₂-C₆ alkynyl, and optionally substituted C₃-C₈ carbocyclyl; or R⁴ and R⁵, together with the atoms to which each is attached, form an optionally substituted C₃-C₆ cycloalkyl; or R⁵ and R⁶, together with the atoms to which each is attached, form an optionally substituted C₃-C₆ cycloalkyl; or R⁴ and R⁶ or R⁵ and R⁷ combine to form an optionally substituted C₁-C₂ alkylene; or R⁴ and R^{4′}, R⁵ and R^{5′}, or R⁶ and R^{6′} combine to form oxo; R⁸ is H or optionally substituted C₁-C₆ alkyl; each of R⁹ and R¹⁰ is, independently, H or methyl; R¹¹ is H; or R⁵ and R¹¹ combine to form a group of formula —Y¹-Y²-Y³—; each of Y¹ and Y² is selected from optionally substituted methylene; optionally substituted ethylene; —CH₂O—; —CH₂NRᵢ; —CH₂NRᵢC(O)—; —CH₂NRᵢS(O)₂—; —CH₂S(O)₂NRᵢ—; —CH₂(4- to 6-membered heterocyclylene)-; —CH₂O(4- to 6-membered heterocyclylene)-, wherein Rᵢ, in each instance, is H or optionally substituted C₁-C₆ alkyl; Y³ is optionally substituted C₁-C₆ alkylene or optionally substituted C₂-C₆ alkenylene; m is 0, 1, or 2; B is optionally substituted C₁-C₆ alkylene, optionally substituted C₂-C₆ alkenylene, optionally substituted C₃-C₁₀ carbocyclylene, optionally substituted C₆-C₁₄ arylene, or optionally substituted 5- to 10-membered heterocyclylene; and all other variables are as defined for formula (I) above.

In some embodiments, said preparing the compound of formula (III) or the pharmaceutically acceptable salt thereof comprises coupling the compound for formula (I) to the compound of formula (IV):

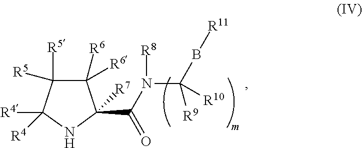

or a salt thereof, wherein all variables are as defined for formula (III). In some embodiments, said preparing the compound of formula (III) or the pharmaceutically acceptable salt thereof includes coupling the compound of formula (I) to the hydrochloride salt of the compound of formula (IV), e.g., in dimethylformamide in the presence of 1-[bis(dimethylamino)methylene]-1H-1,2,3-triazolo[4,5-b]pyridinium 3-oxide hexafluorophosphate and N,N-diisopropylethylamine. In some embodiments, said preparing the compound of formula (III) or the pharmaceutically acceptable salt thereof includes coupling the compound of formula (I) to the hydrobromide salt of the compound of formula (IV), e.g., in acetonitrile in the presence of propanephosphonic acid anhydride and N,N-diisopropylethylamine. In some embodiments, said preparing the compound of formula (III) or the pharmaceutically acceptable salt thereof includes coupling the compound of formula (I) to the trifluoroacetic acid salt of the compound of formula (IV), e.g., in dimethylformamide in the presence of N, N-diisopropylethylamine and 1-[bis(dimethylamino)-methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

In some embodiments, R⁸ is H.

In some embodiments, R⁸ is CH₃.

In some embodiments, m is 1.

In some embodiments, m is 2.

In some embodiments, m is 0.

In some embodiments, each of R⁹ and R¹⁰ is H.

In some embodiments, R⁹ is H and R¹⁰ is CH₃.

In some embodiments, each of R⁹ and R¹⁰ is CH₃.

In some embodiments, R⁵ is fluoro. In some embodiments, R⁵ is fluoro and each of R⁴, R^{4′}, R⁶, R^{6′}, and R⁷ is hydrogen, e.g.,

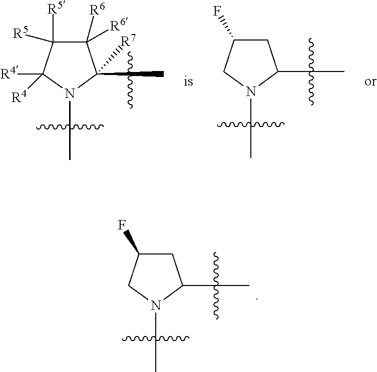

In some embodiments,

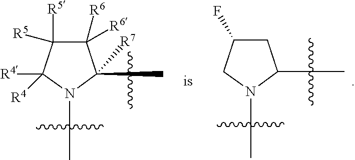

In some embodiments, R⁵ is fluoro and R^{5′} is optionally substituted C₁-C₆ alkyl, e.g.,

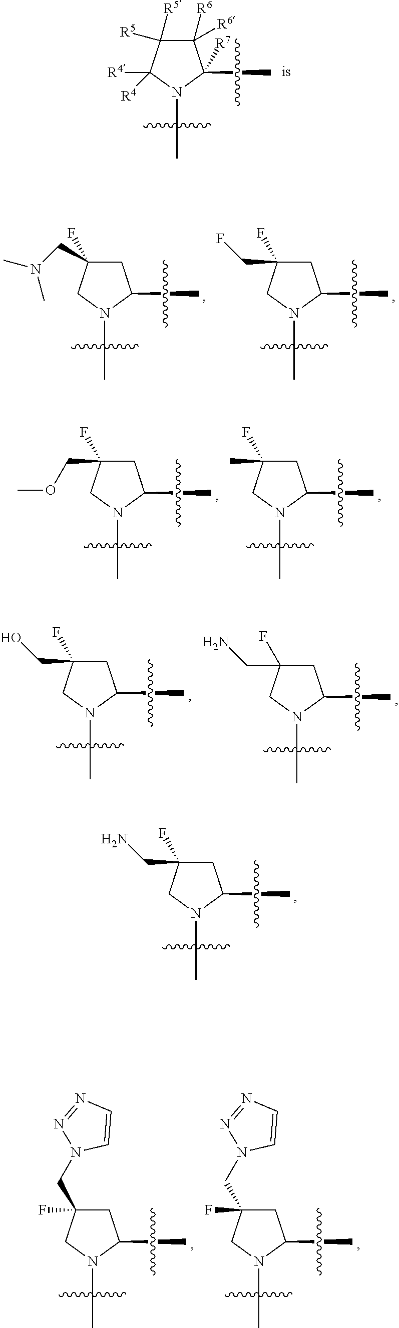

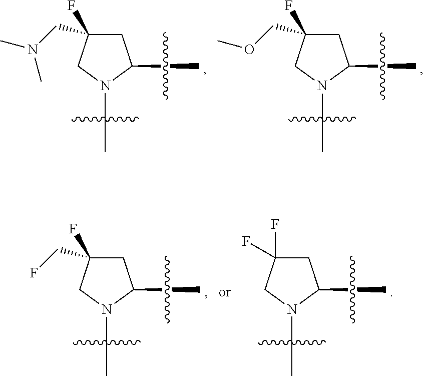

In some embodiments in which R⁵ is fluoro,

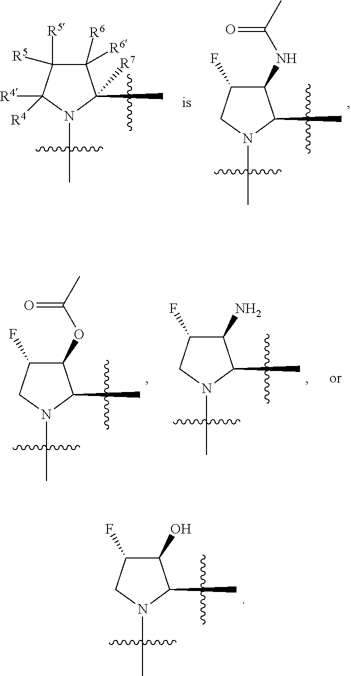

In some embodiments, R⁴ and R⁵, together with the atoms to which each is attached, form an optionally substituted C₃-C₆ cycloalkyl (e.g., optionally substituted cyclopropyl); and each of R⁴, R⁶, R^{6′}, and R⁷ is H. In some embodiment, R^{5′} is H, e.g.,

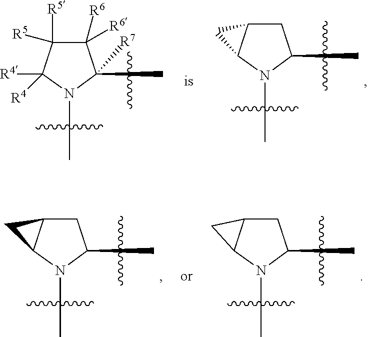

In some embodiments, R^{5′} is optionally substituted C₁-C₆ alkyl, e.g.,

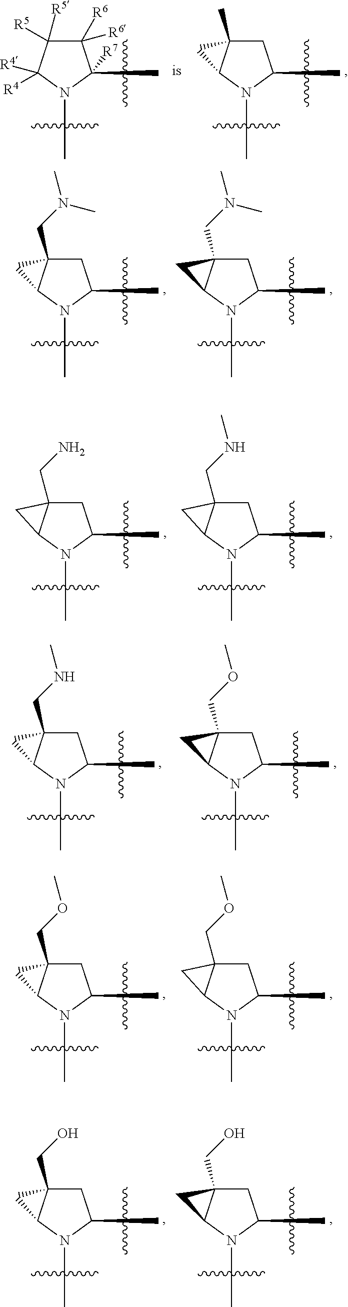

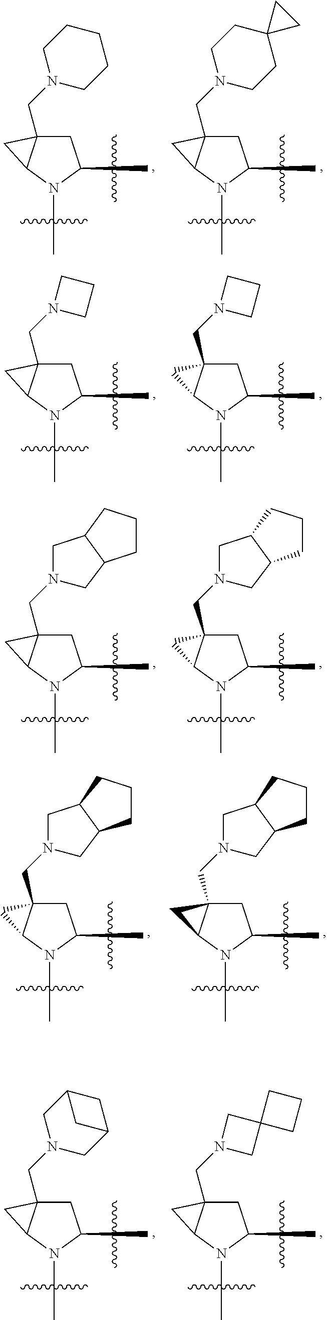

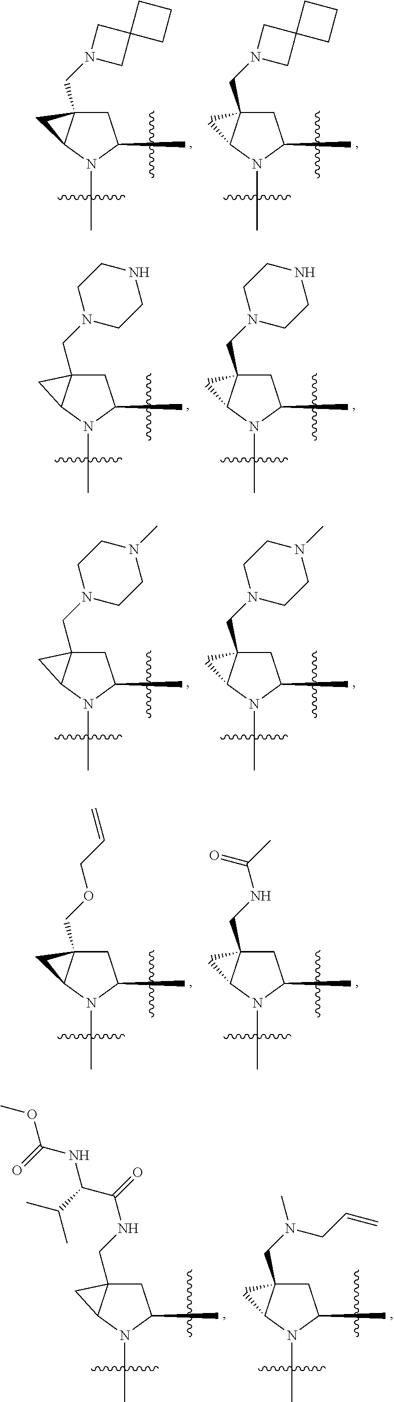

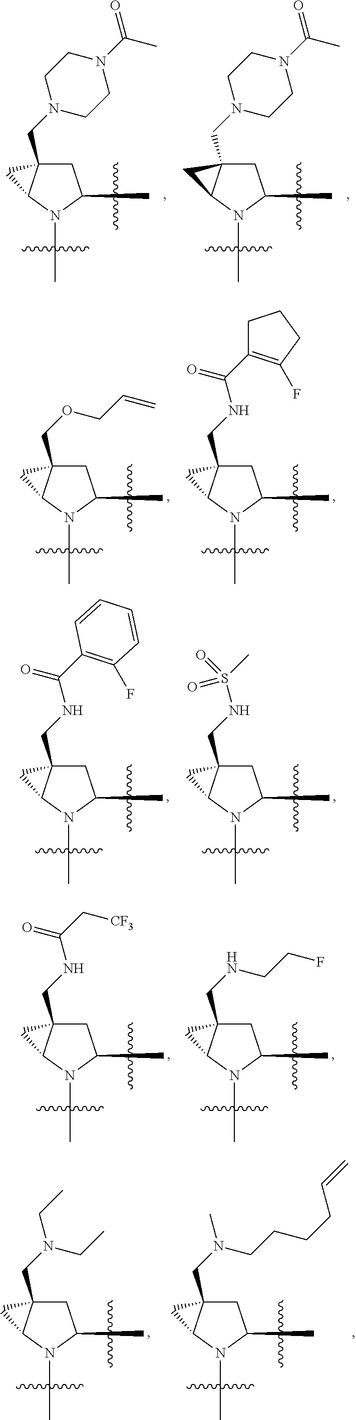

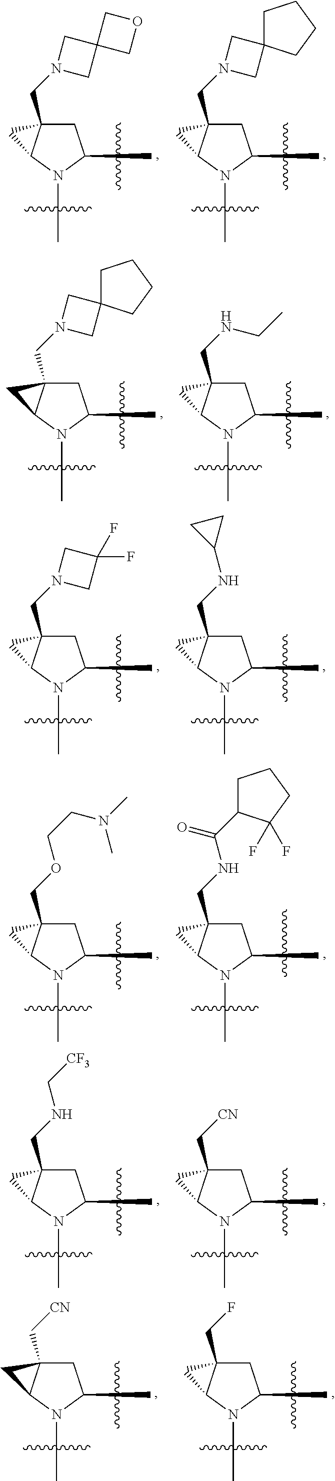

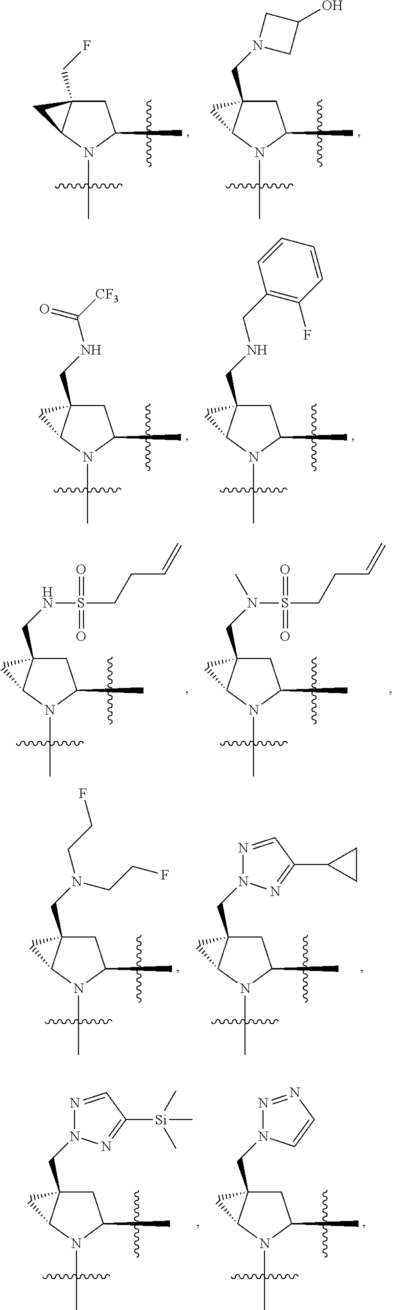

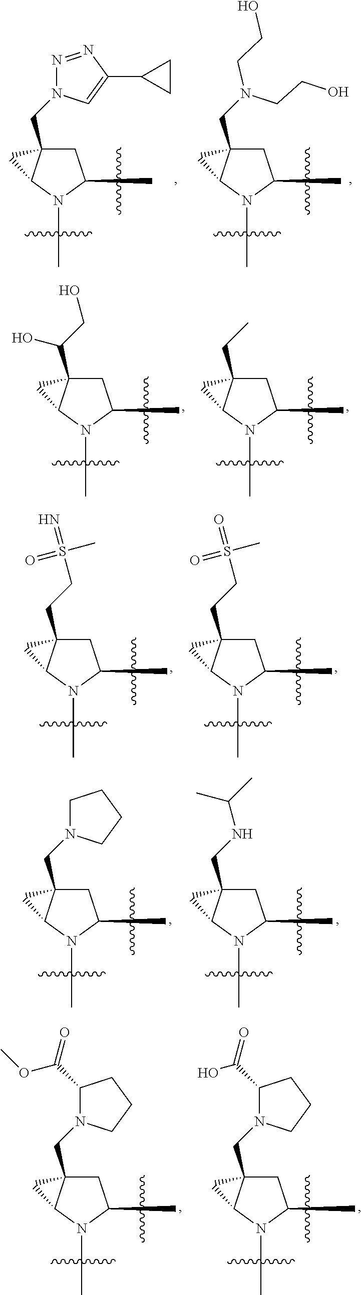

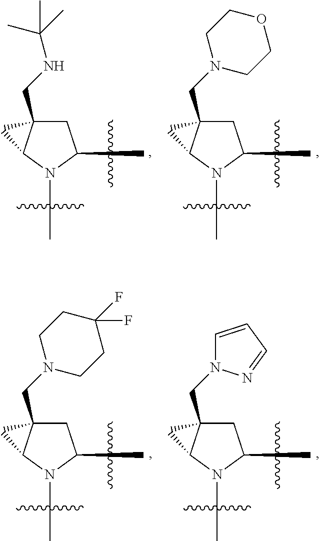

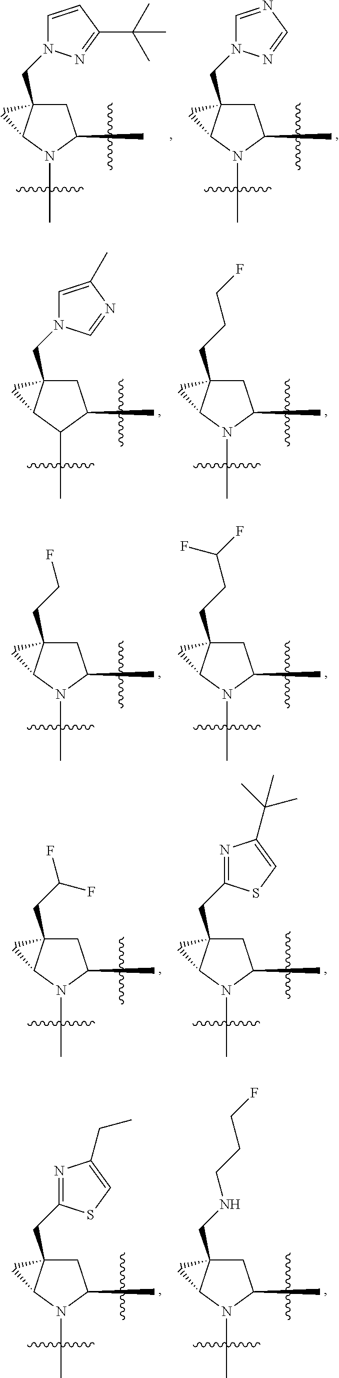

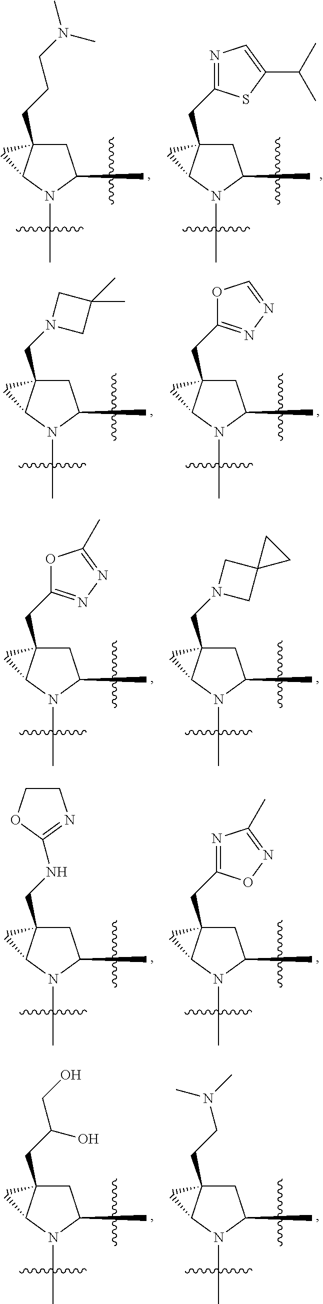

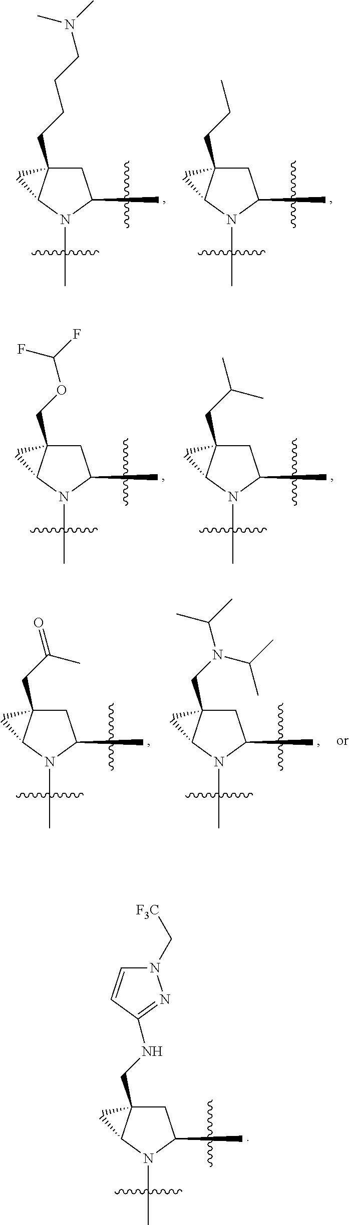

In some embodiments

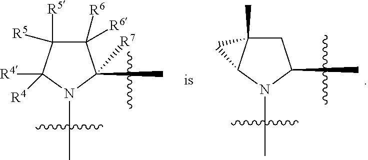

In some embodiments,

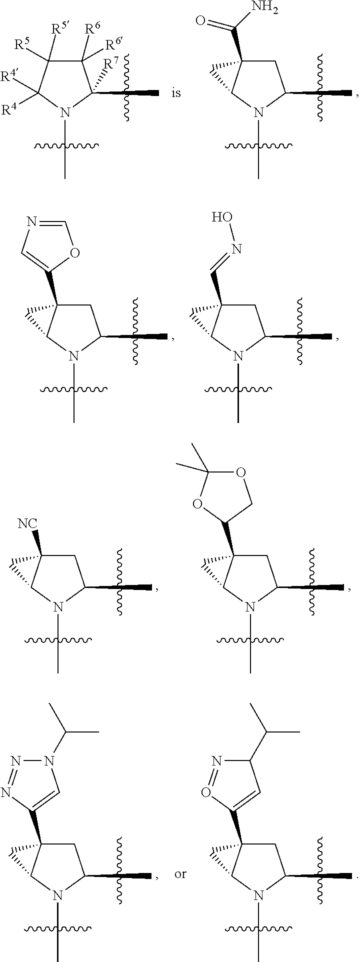

In some embodiments, R⁵ and R⁶, together with the atoms to which each is attached, form an optionally substituted C₃-C₆ cycloalkyl (e.g., optionally substituted cyclopropyl), and each of R⁴, R^{4′}, R^{5′}, R^{6′}, and R⁷ is H, e.g.,

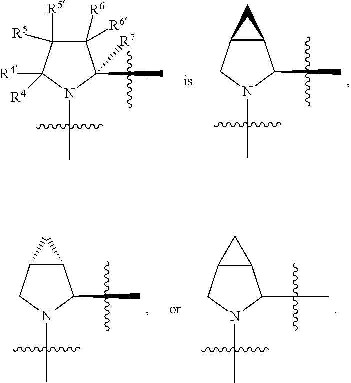

In some embodiments, R⁵ and R⁷ combine to form optionally substituted C₁-C₂ alkylene, e.g.,

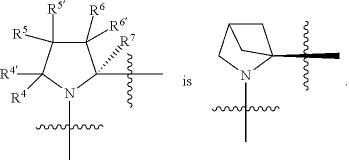

In some embodiments,

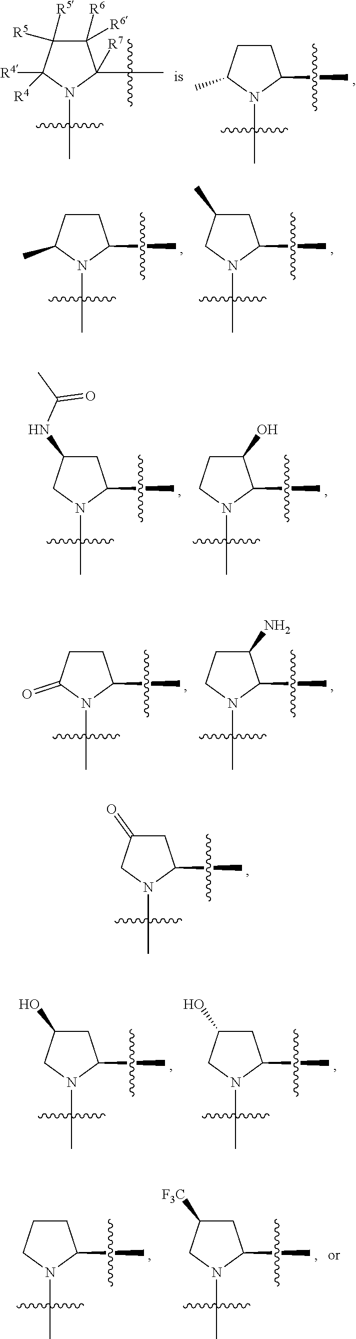

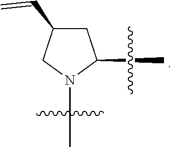

In some embodiments, BR¹¹ is optionally substituted 5- to 10-membered heteroaryl, such as 6-membered heteroaryl, e.g., optionally substituted pyridyl, optionally substituted pyridazinyl, optionally substituted pyrimidinyl, or optionally substituted pyrazinyl.

In some embodiments, BR¹¹ is optionally substituted pyridyl, e.g.,

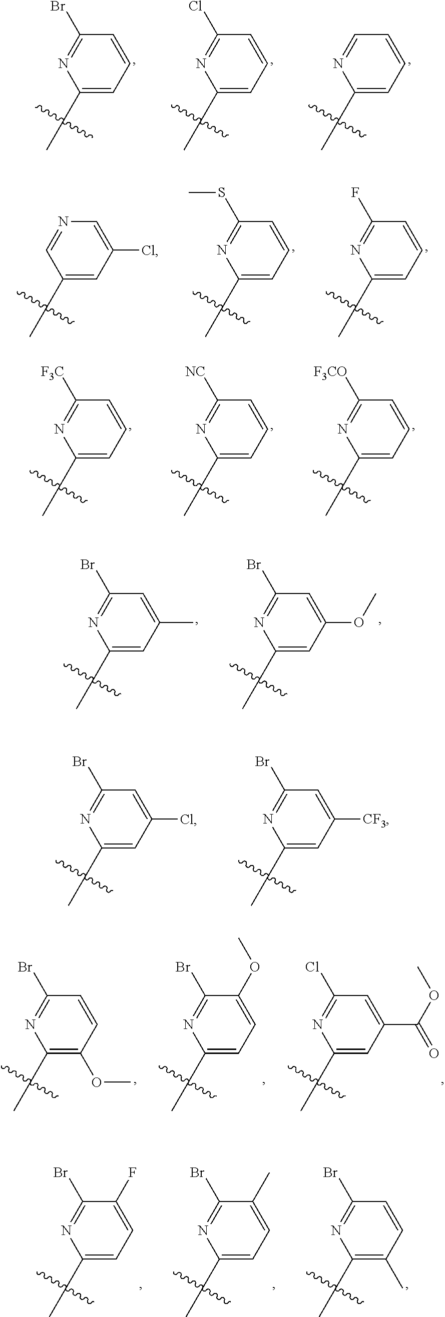

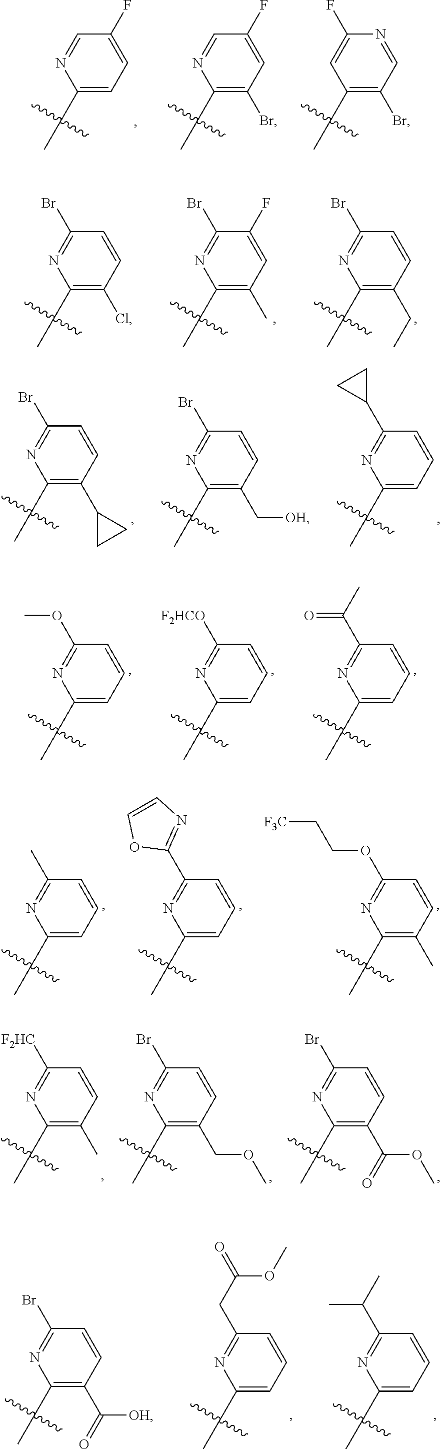

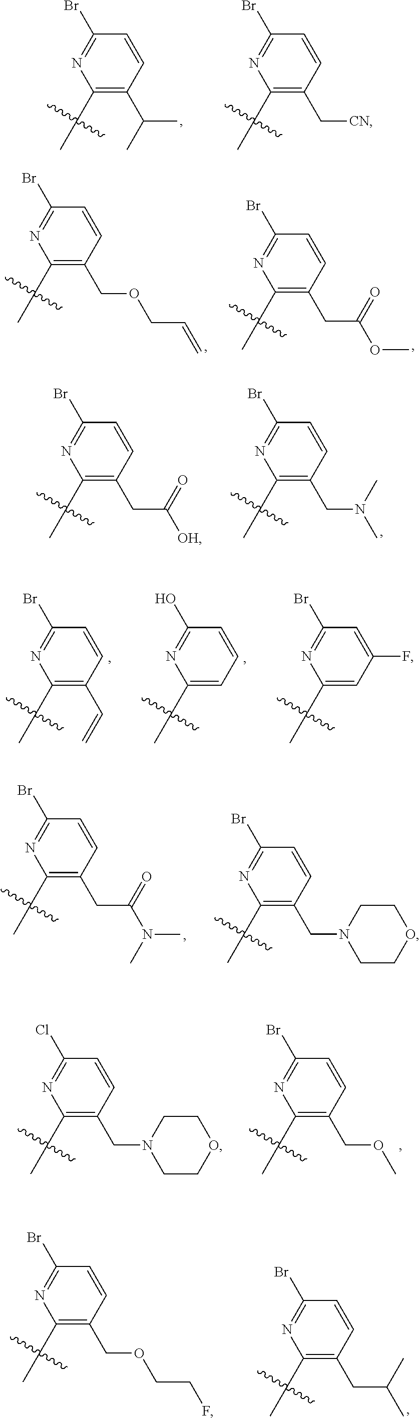

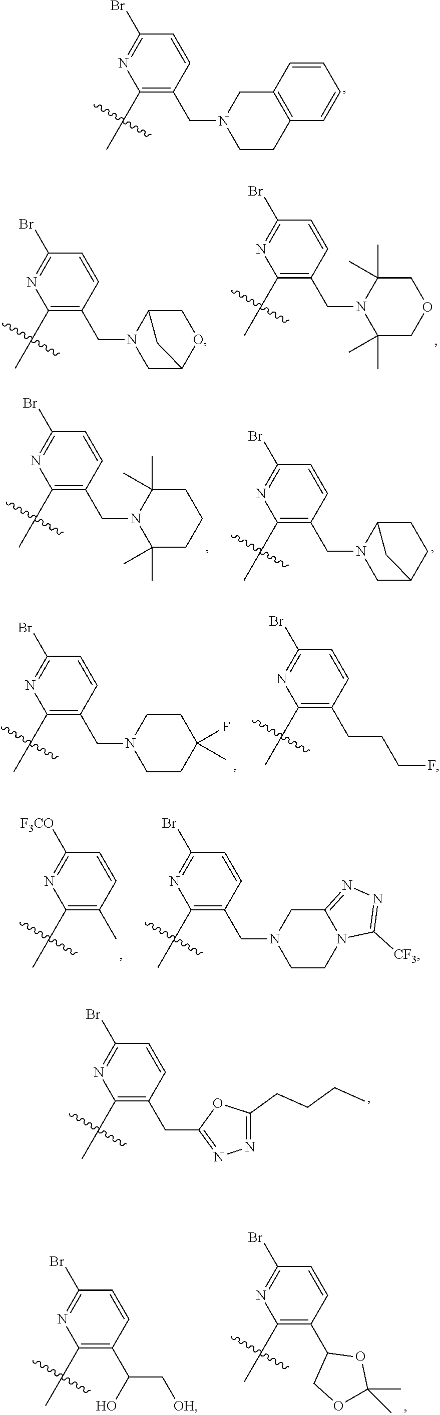

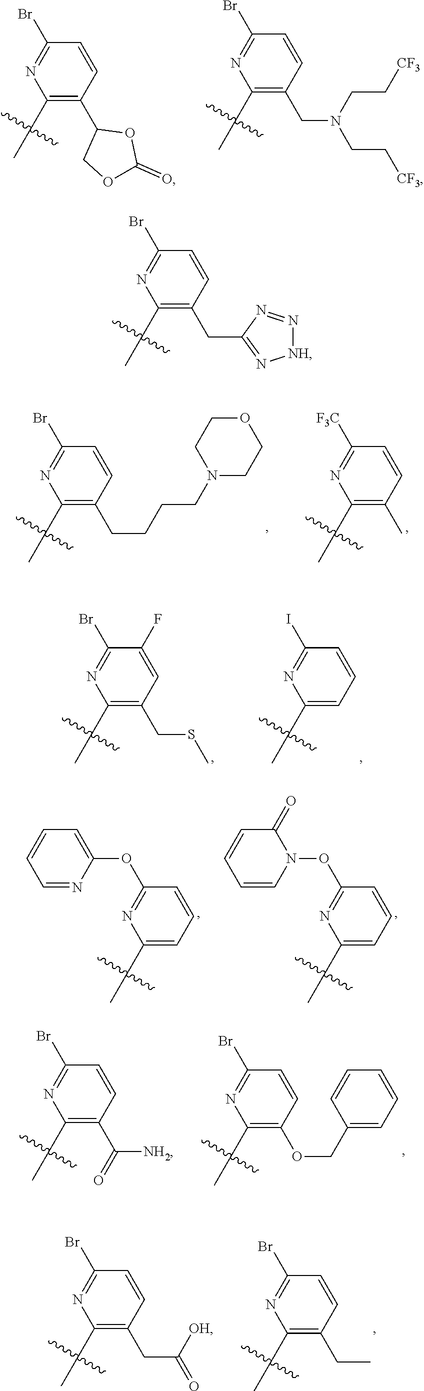

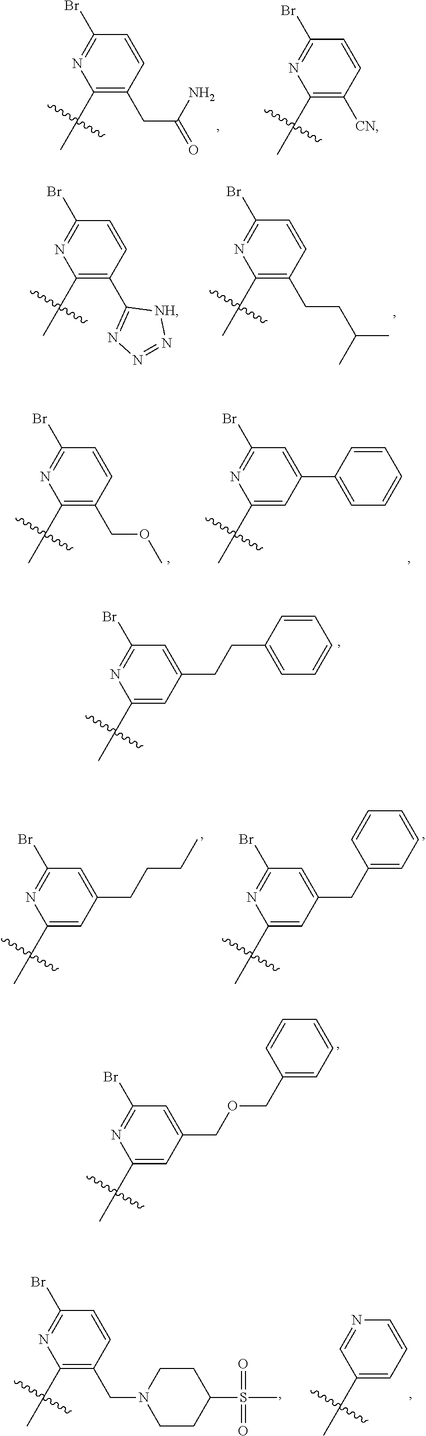

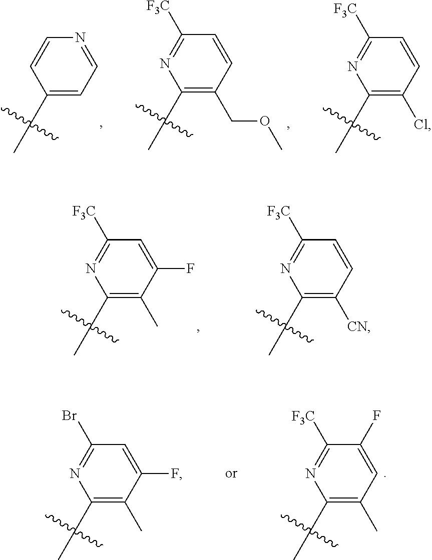

In some embodiments, BR¹¹ is

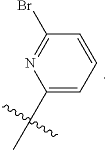 [SMILES: *c1cccc(Br)n1]

In some embodiments, BR¹¹ is w

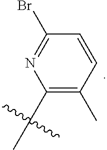 [SMILES: *c1nc(Br)ccc1C]

In some embodiments, BR¹¹ is optionally substituted pyrazinyl, e.g.,

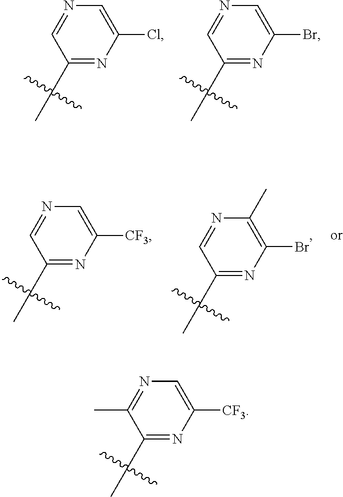

In some embodiments, BR¹¹ is optionally substituted pyrimidinyl, e.g.,

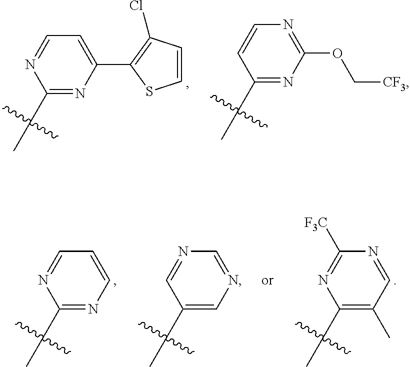 [SMILES: *c1nccc(-c2sccc2Cl)n1 ; *c1ccnc(OCC(F)(F)F)n1 ; *c1ncccn1 ; *c1cncnc1 ; *c1nc(C(F)(F)F)ncc1C]

In some embodiments, BR¹¹ is optionally substituted pyridazinyl, e.g.,

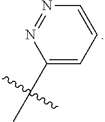 [SMILES: *c1cccnn1]

In some embodiments, BR¹¹ is optionally substituted five-membered heteroaryl, e.g.,

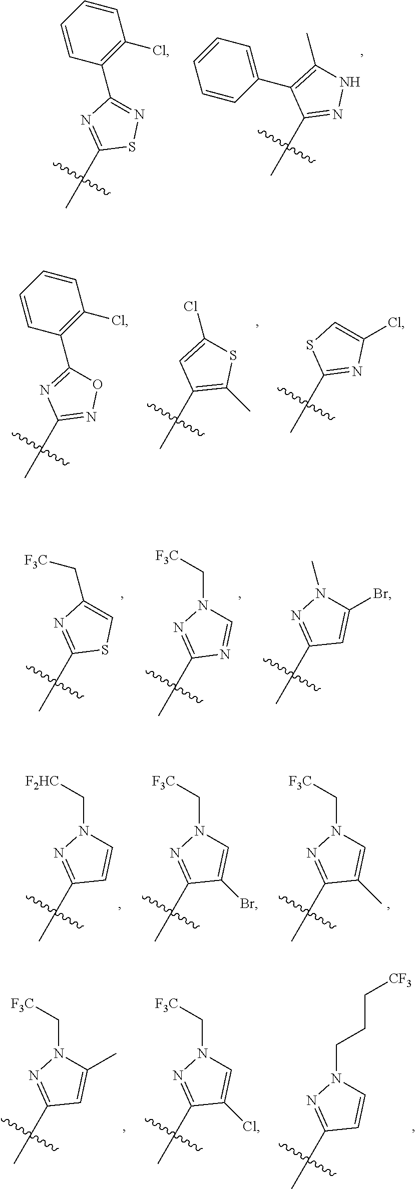

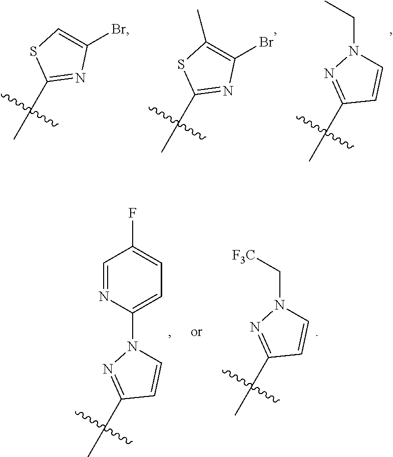

In some embodiments, BR¹¹ is bicyclic 9- or 10-membered bicyclic heteroaryl, e.g.,

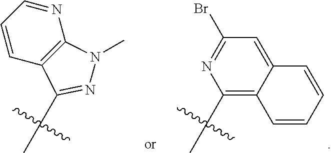 [SMILES: *c1nn(C)c2ncccc12 ; *c1nc(Br)cc2ccccc12]

In some embodiments, BR¹¹ is optionally substituted C₆-C₁₄ aryl, such as optionally substituted phenyl, e.g.

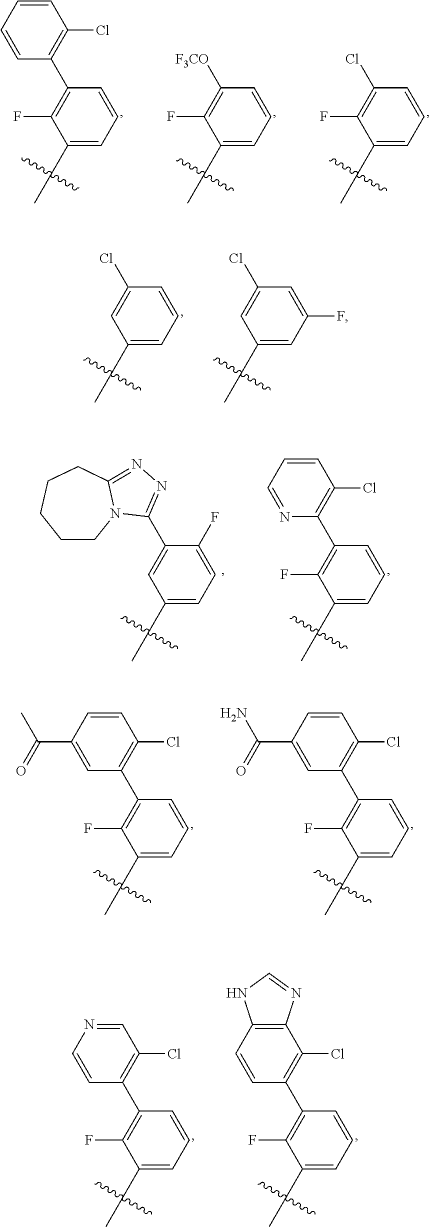

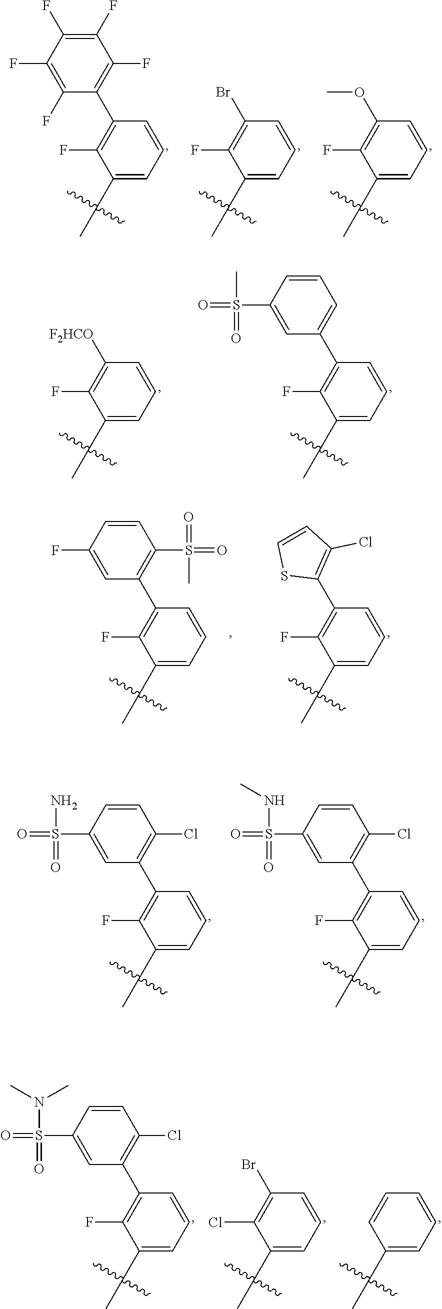

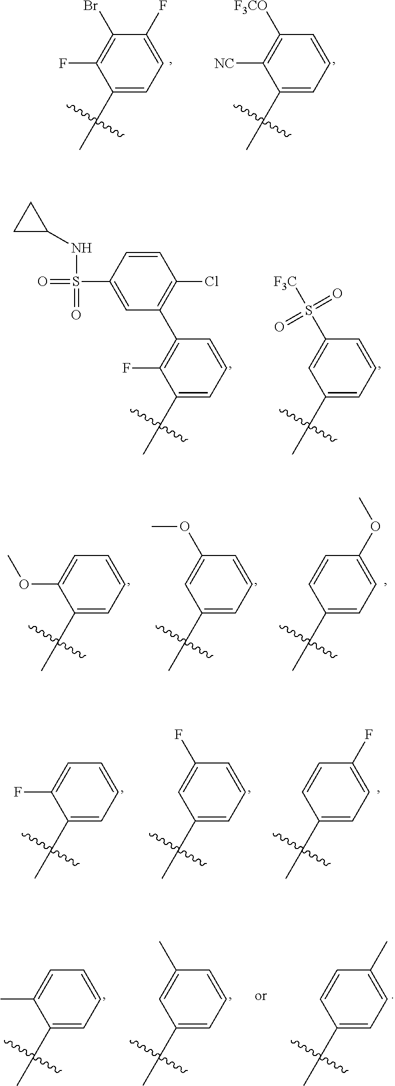

In some embodiments, BR¹¹ is optionally substituted 5- to 9-membered unsaturated heterocyclyl, e.g.,

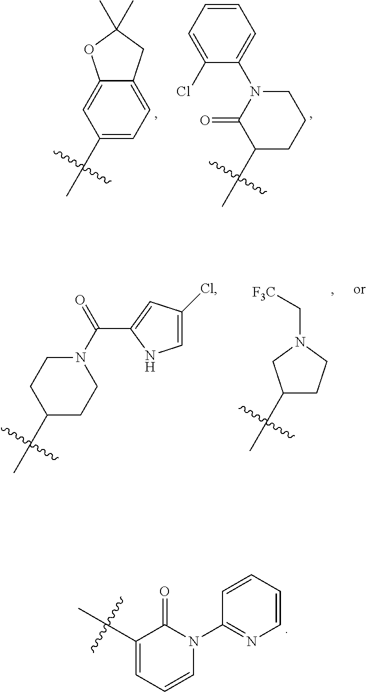

In some embodiments, BR¹¹ is optionally substituted C₃-C₁₀ cycloalkyl, e.g.,

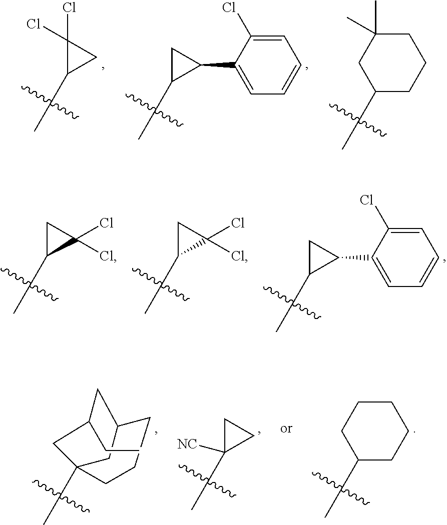

In some embodiments, BR¹¹ is optionally substituted C₂-C₆ alkenyl, e.g.,

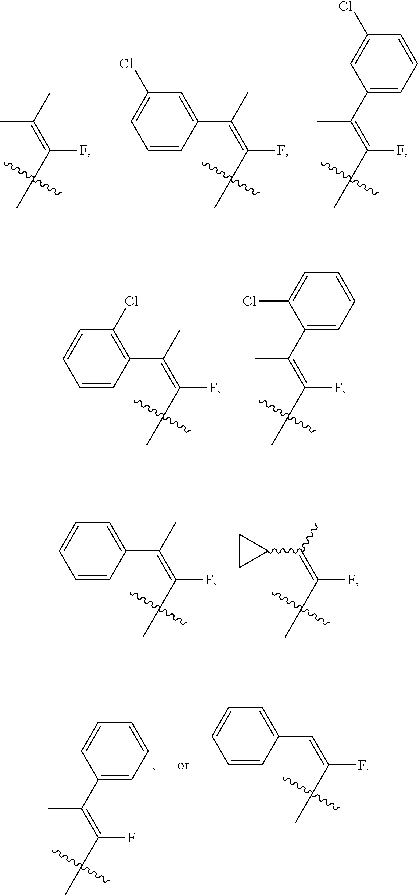

In some embodiments, BR¹¹ is optionally substituted C₁-C₆ alkyl, e.g.,

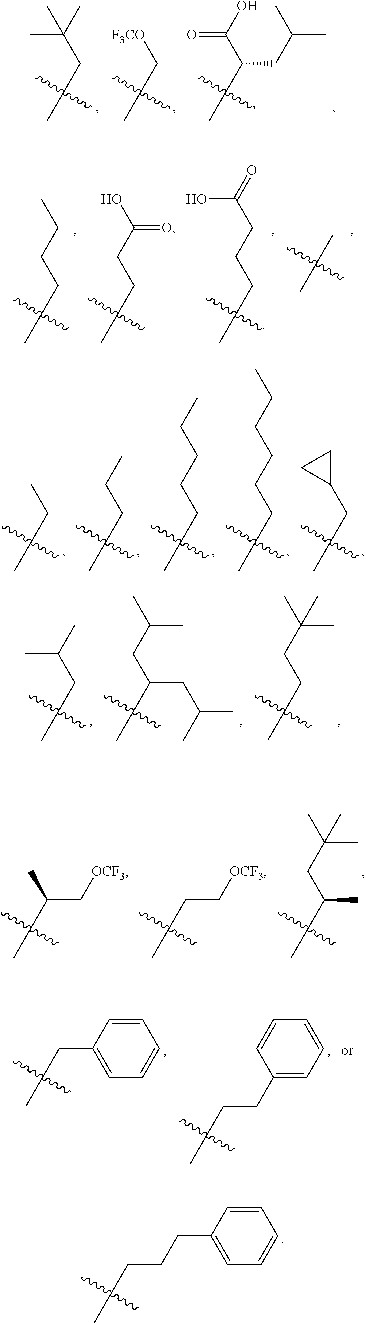

In some embodiments, R⁵ and R¹¹ combine to form a group of formula —Y¹-Y²-Y³—, e.g.,

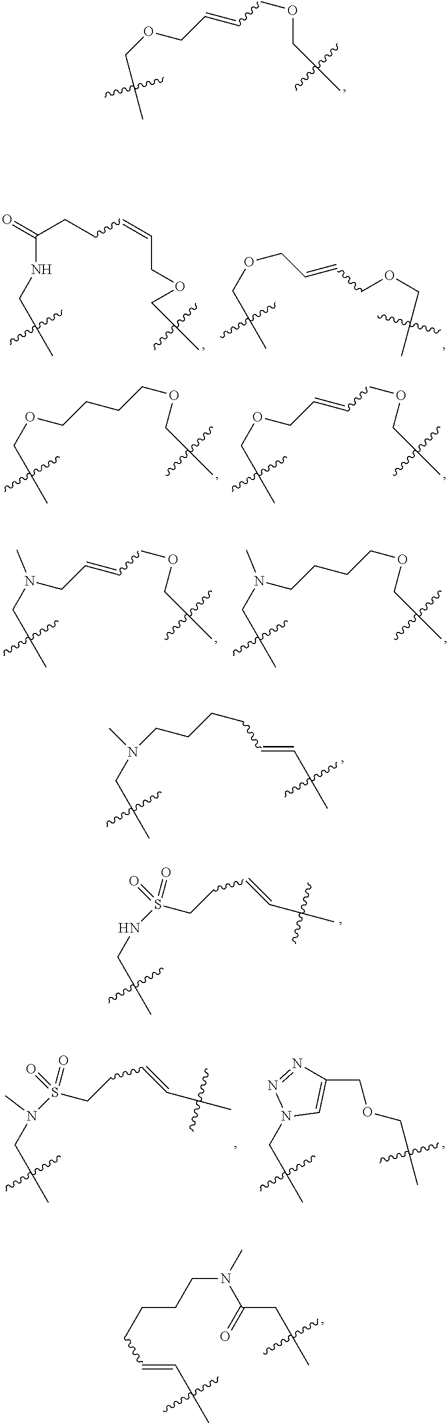

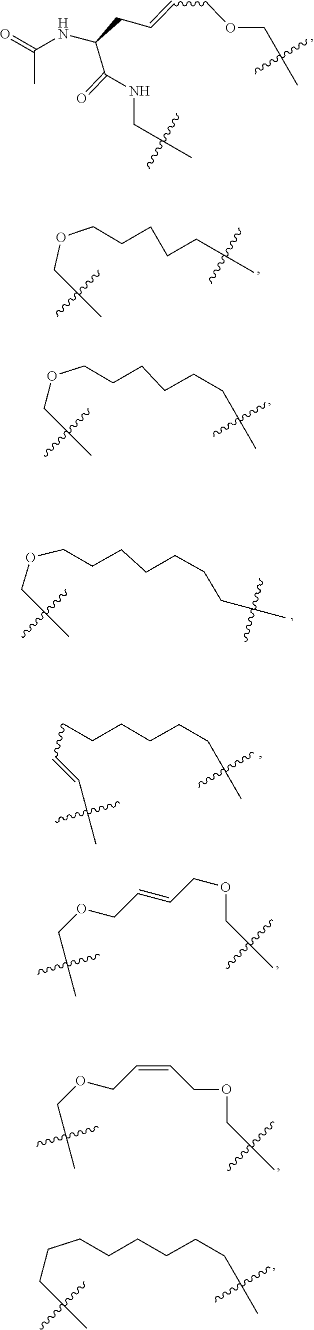

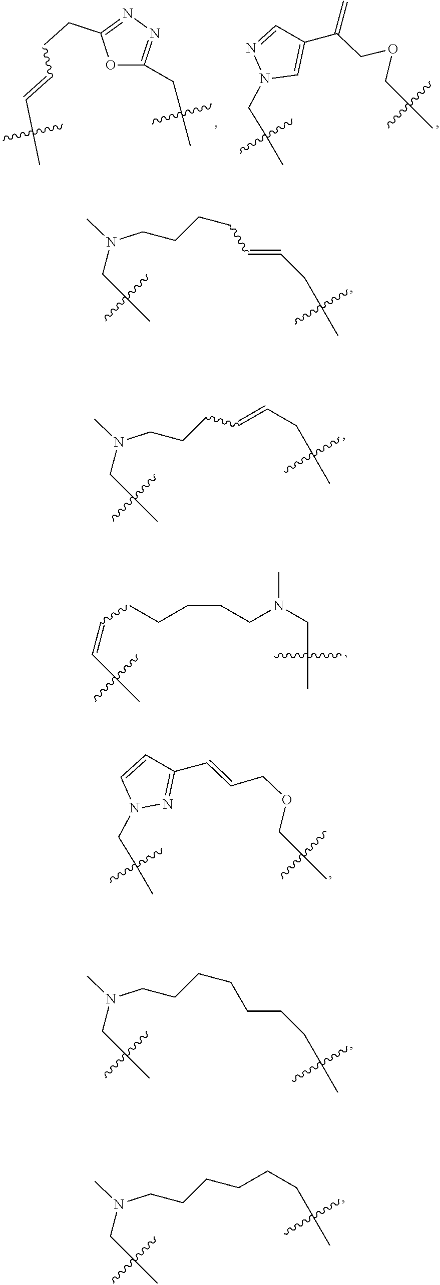

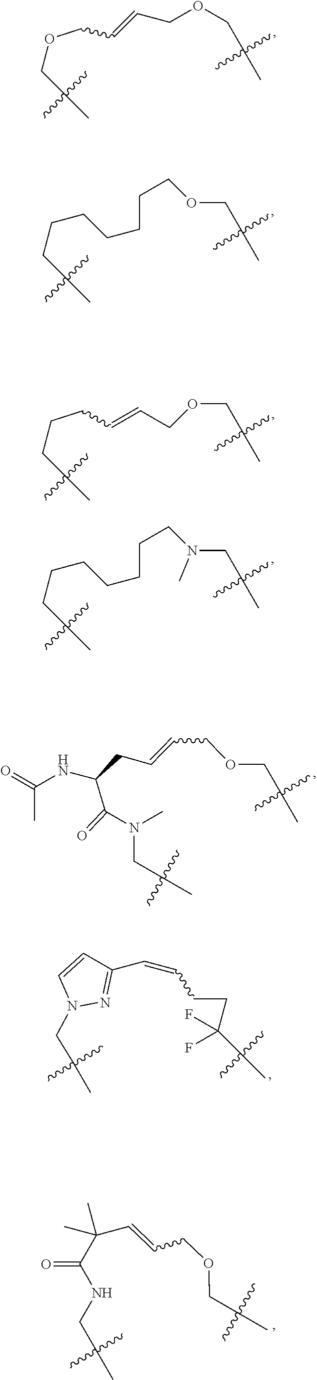

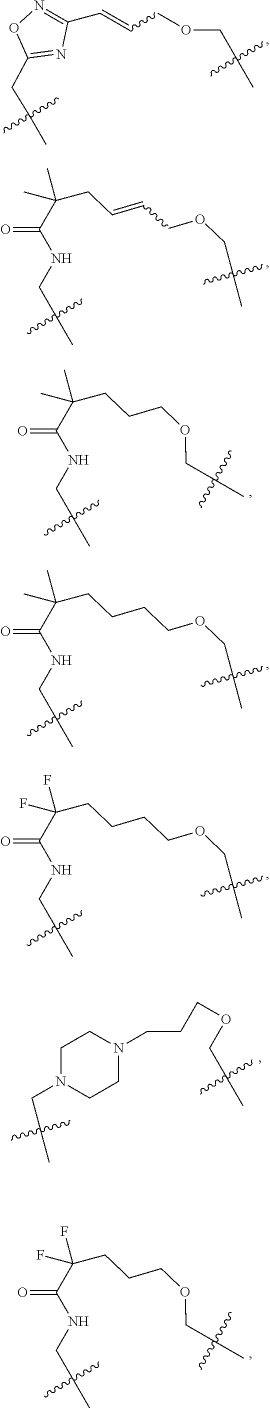

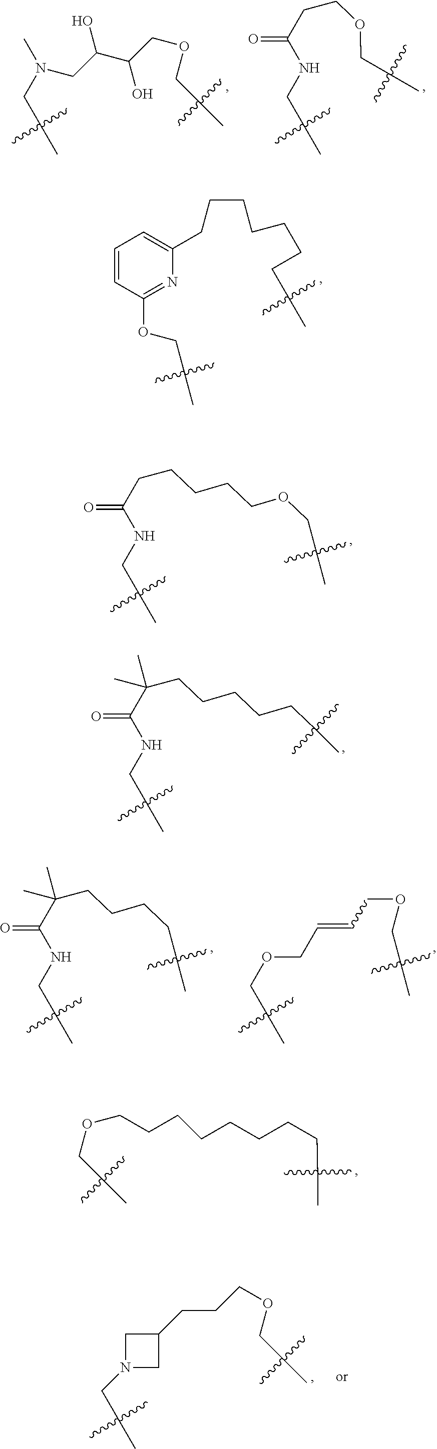

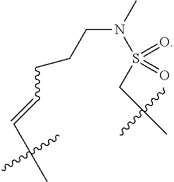 [SMILES: *C=CCCCN(C)S(=O)(=O)C*]

In some embodiments,

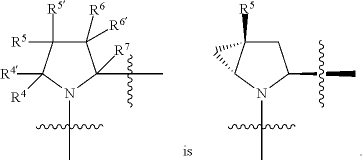

In some embodiments, is

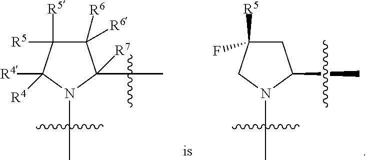

In some embodiments, BR¹¹ is

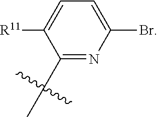

In some embodiments, BR¹¹ is

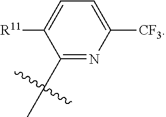

In some embodiments, X¹ is N.

In some embodiments, X¹ is CR_{c}, e.g., C(CH₃) or CH.

In some embodiments, X² is CR_{d}. In some embodiments, R_{d} is H or optionally substituted C₁-C₆ alkyl, e.g., X² is CH or C(CH₃).

In some embodiments, X⁵ is CR_{d}, e.g., CH.

In some embodiments, X³ is CR_{f}, e.g., X³ is CR_{f} and X⁴ is N or X³ is CR_{f} and X⁴ is CRₑ, e.g., CH.

In some embodiments, X⁴ is CR_{f}, e.g., X⁴ is CR_{f} and X³ is N or X⁴ is CR_{f} and X³ is CRₑ, e.g., CH.

In some embodiments, R_{f} is optionally substituted 5- to 10-membered heteroaryl containing 1, 2, or 3 heteroatoms selected from N, O, and S, e.g., 6-membered heteroaryl containing 1, 2, or 3 heteroatoms selected from N, O, and S.

In some embodiments, R_{f} is optionally substituted pyrimidinyl, e.g.,

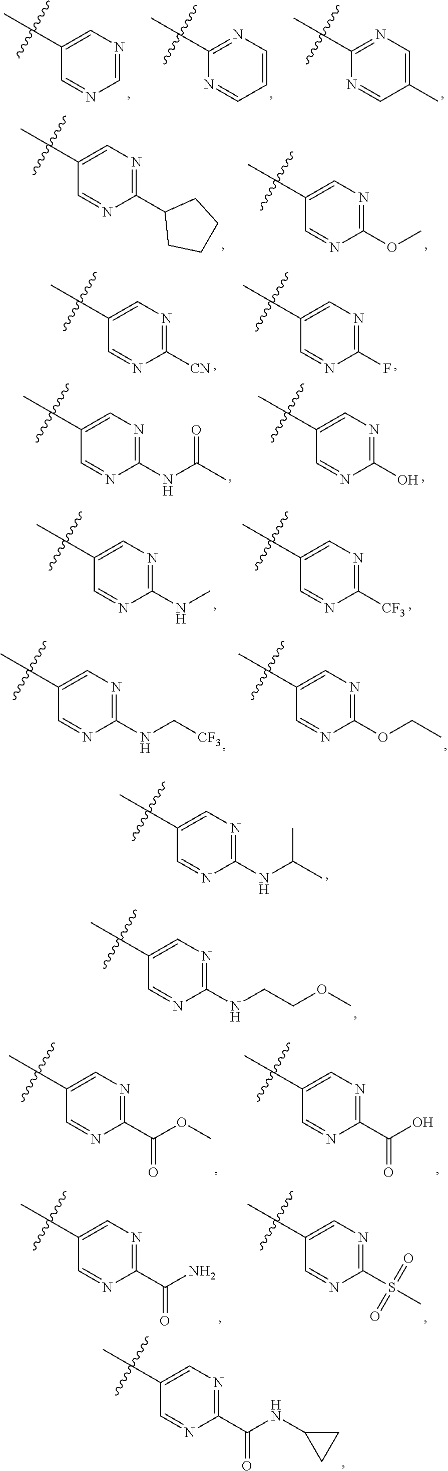

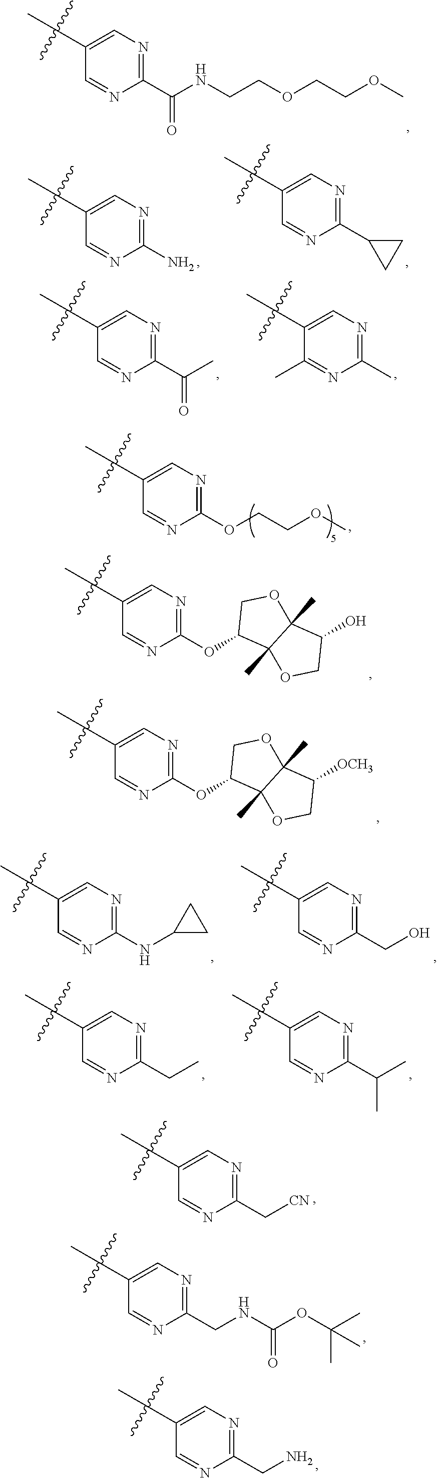

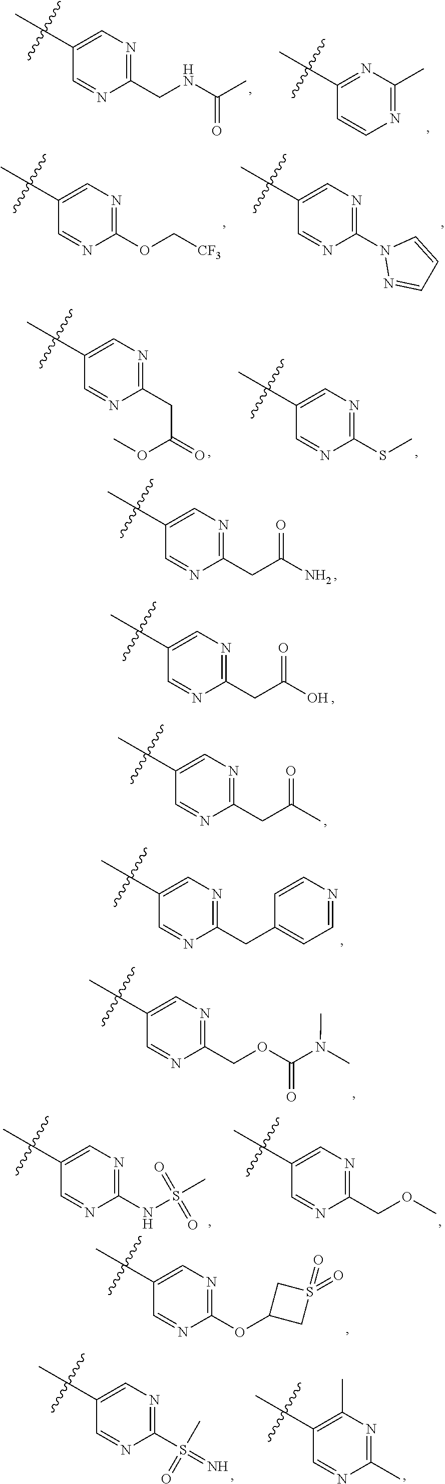

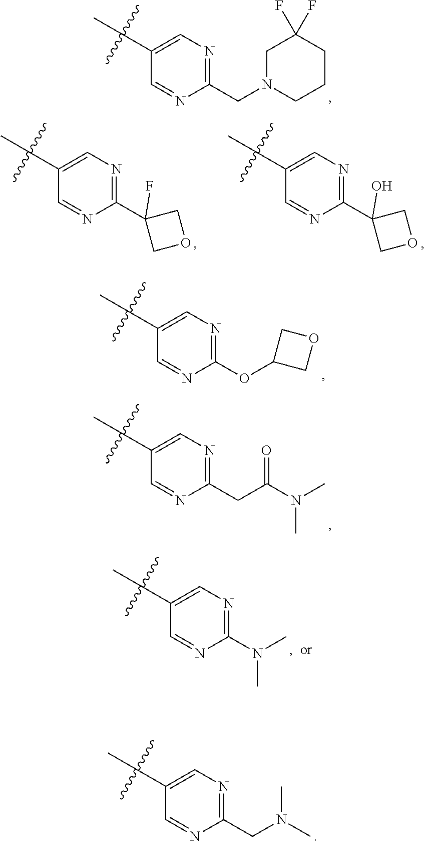

In some embodiments, R_{f} is

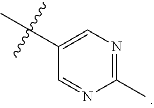 [SMILES: *c1cnc(C)nc1]

In some embodiments, R_{f} is

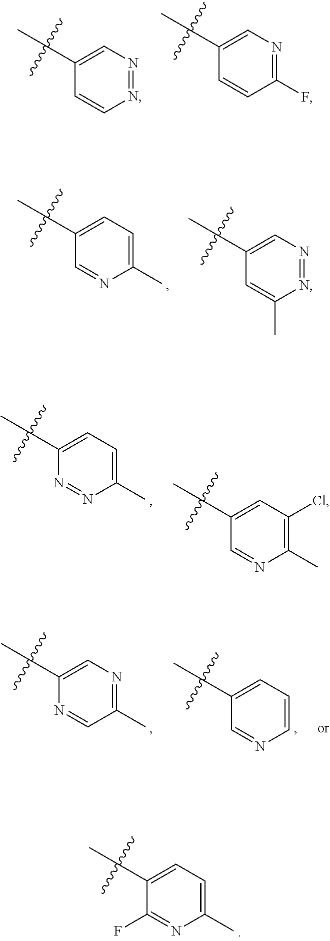

In some embodiments, R_{f} is optionally substituted 8- to 10-membered bicyclic heteroaryl containing 1, 2, or 3 heteroatoms selected from N, O, and S, e.g., R_{f} is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl, e.g.,

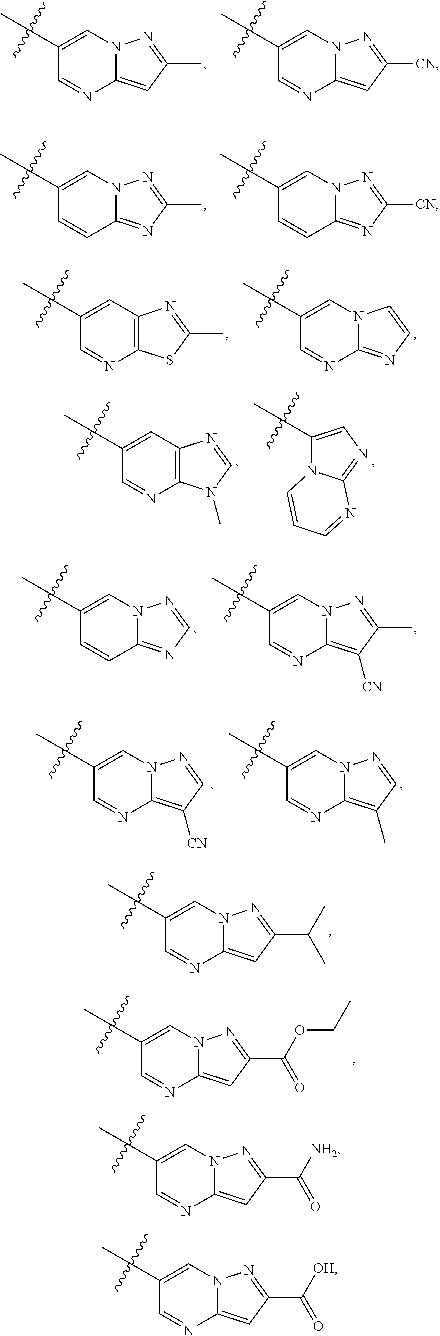

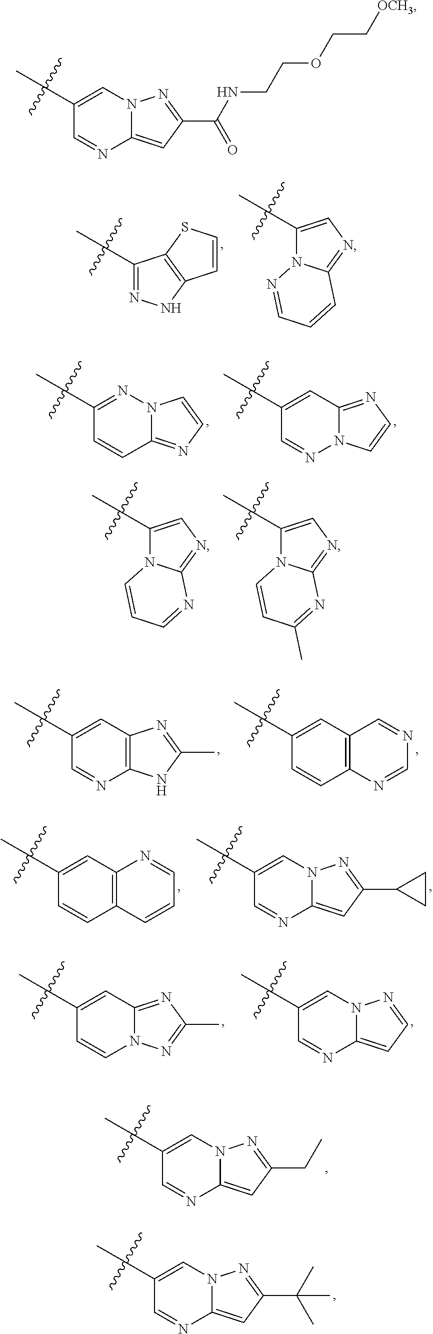

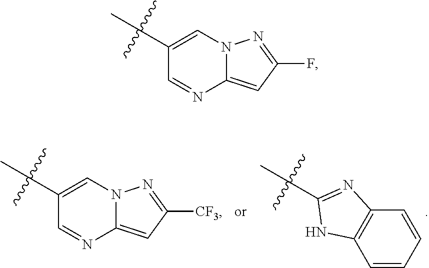 [SMILES: *c1cnc2cc(F)nn2c1 ; *c1cnc2cc(C(F)(F)F)nn2c1 ; *c1nc2ccccc2[nH]1]

In some embodiments, R_{f} is

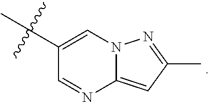 [SMILES: *c1cnc2cc(C)nn2c1]

In some embodiments, R_{f} is

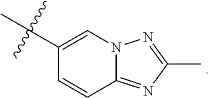 [SMILES: *c1ccc2nc(C)nn2c1]

In some embodiments, R_{f} is optionally substituted C₆-C₁₄ aryl. For example, R_{f} is optionally substituted phenyl, e.g.,

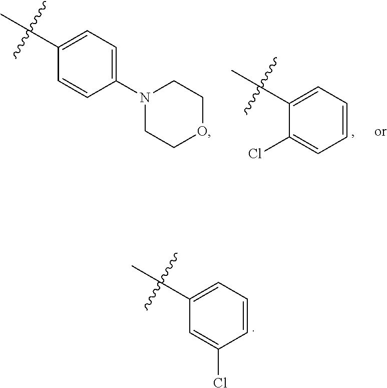 [SMILES: *c1ccc(N2CCOCC2)cc1 ; *c1ccccc1Cl ; *c1cccc(Cl)c1]

In some embodiments, R_{f} is optionally substituted 6- to 9-membered unsaturated heterocyclyl containing 1-4 heteroatoms selected from N, O, or S. In some embodiments, R_{f} is bonded to the carbon atom to which it is attached through a carbon ring atom contained therein, e.g., R_{f} is

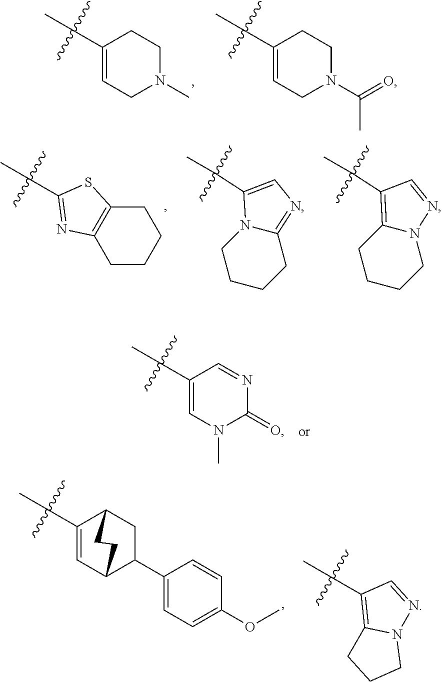

In some embodiments, R_{f} is

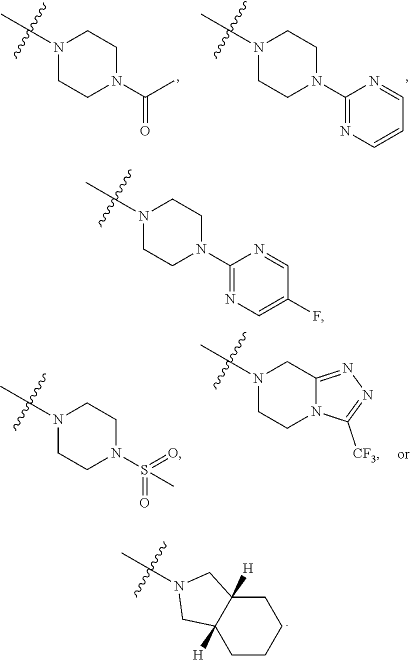

In some embodiments, R_{f} is optionally substituted 5-membered heteroaryl containing 1, 2, or 3 heteroatoms selected from N, O, and S, e.g.,

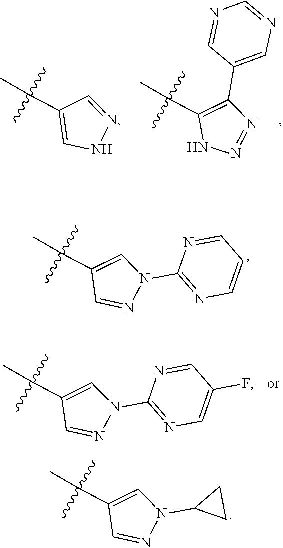 [SMILES: *c1cn[nH]c1 ; *c1[nH]nnc1-c1cncnc1 ; *c1cnn(-c2ncccn2)c1 ; *c1cnn(-c2ncc(F)cn2)c1 ; *c1cnn(C2CC2)c1]

In some embodiments, R¹ is —C(O)R_{b}, e.g.,

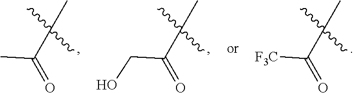

In some embodiments, R¹ is

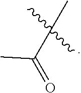 [SMILES: *C(C)=O]

In some embodiments, R¹ is —C(O)NRₐRₐ′, e.g.,

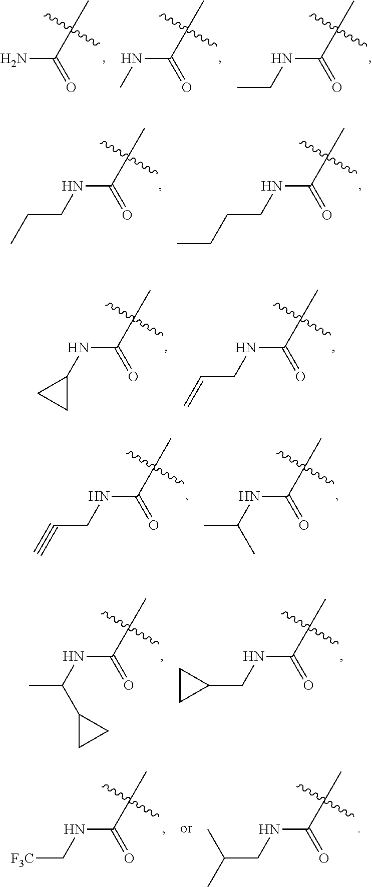

In some embodiments, R¹ is

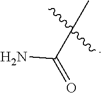 [SMILES: *C(N)=O]

In some embodiments, R¹ is —C(O)OR_{b}, e.g., —C(O)OCH₃ or —C(O)OH.

In some embodiments, R¹ is optionally substituted C₁-C₆ alkyl, e.g.,

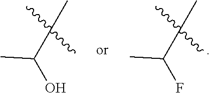

In some embodiments, R¹ is

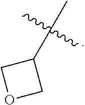 [SMILES: *C1COC1]

In some embodiments, R¹ is cyano.

In some embodiments, R¹ is halo.

In some embodiments, R² is H.

In some embodiments, R³ is H.

In some embodiments, the compound of formula (III) is:

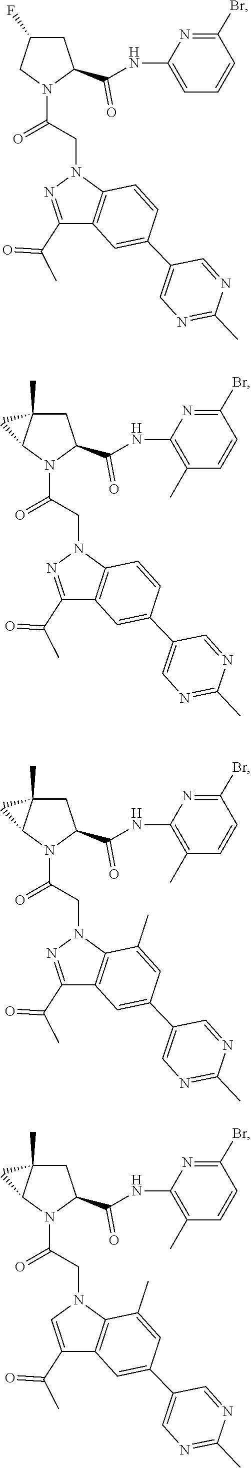

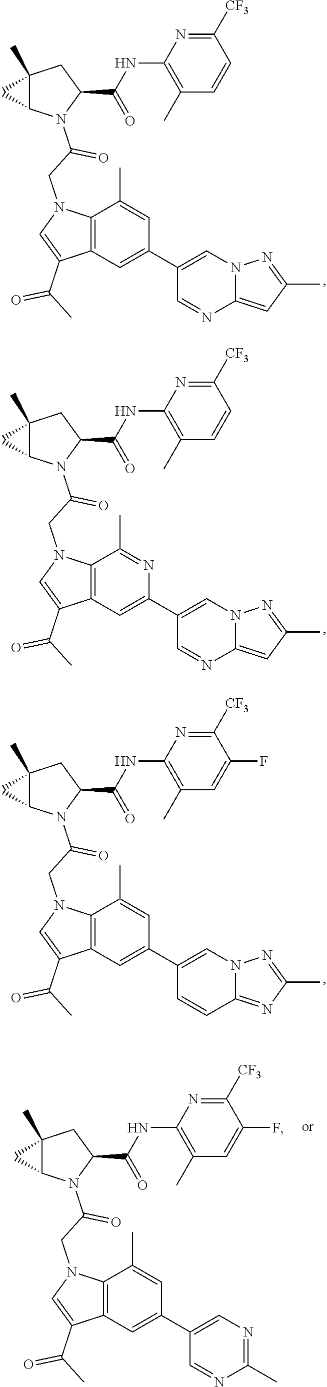

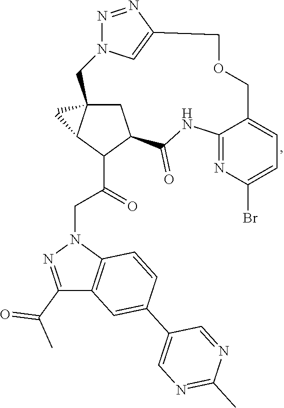 [SMILES: CC(=O)c1nn(CC(=O)C2[C@H]3C[C@]4(C[C@@H]24)Cn2cc(nn2)COCc2ccc(Br)nc2NC3=O)c2ccc(-c3cnc(C)nc3)cc12]

pharmaceutically acceptable salt thereof.

In some embodiments, the weak base is at least one of potassium carbonate or cesium carbonate. For example, the weak base is potassium carbonate.

In some embodiments, the water miscible organic solvent is selected from 1,2-propanediol, dimethylformamide, di-isopropylethylamine, or dimethyl sulfoxide. For example, the water miscible organic solvent is 1,2-propanediol.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-12, 1-10, 1-8, 1-6, 1-4, or 1-2 carbon atoms (e.g., C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl.

The term “alkylene,” as used herein, refers to a divalent radical obtained by removing a hydrogen atom from a carbon atom of an alkyl group. The divalency of an alkylene group does not include the optional substituents on the alkylene group.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkenylene,” as used herein, refers to a divalent radical obtained by removing a hydrogen atom from a carbon atom of an alkenyl group. The divalency of an alkenylene group does not include the optional substituents on the alkenylene group.

The term “alkenyloxy,” as used here, refers to a monovalent radical having the structure —O-alkenyl, in which “alkenyl” is as defined herein. Examples include, but are not limited to ethenyloxy, propenyloxy, and the like.

The term “alkoxy,” as used here, refers to a monovalent radical having the structure —O-alkyl, in which “alkyl” is as defined herein. Examples include, but are not limited to methoxy, ethoxy, and n-butoxy, i-butoxy, t-butoxy, and the like.

The term “alkynyl,” as used herein, refers to a branched or straight-chain monovalent unsaturated aliphatic radical containing at least one carbon-carbon triple bond and only C and H when unsubstituted. Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethynyl, 1-propynyl, and 3-butynyl.

The term “aryl,” as used herein, refers to a monocyclic or fused ring bicyclic or polycyclic system which has the characteristics of aromaticity in terms of electron distribution throughout the ring system, e.g., phenyl, naphthyl, or phenanthryl. An aryl group may have, e.g., six to sixteen carbons (e.g., C₆-C₁₆ aryl, C₆-C₁₄ aryl, C₆-C₁₃ aryl, or C₆-C₁₀ aryl).

The term “arylene,” as used herein, refers to a divalent radical obtained by removing a hydrogen atom from a carbon atom of an aryl group. The divalency of an arylene group does not include the optional substituents on the alkenylene group.

The term “carbocyclyl,” as used herein, represents a monovalent, saturated or unsaturated non-aromatic cyclic group containing only C and H when unsubstituted. A carbocyclyl (e.g., a cycloalkyl or a cycloalkenyl) may have, e.g., three to fourteen carbons (e.g., a C₃-C₇, C₃-C₈, C₃-C₉, C₃-C₁₀, C₃-C₁₁, C₃-C₁₂, C₃-C₁₄ carbocyclyl). The term “carbocyclyl” also includes bicyclic and polycyclic (e.g., tricyclic and tetracyclic) fused ring structures.

The term “carbocyclyene,” as used herein, refers to a divalent radical obtained by removing a hydrogen atom from a carbon atom of a carbocyclyl group. The divalency of a carbocyclylene group does not include the optional substituents on the carbocyclylene group

The term “carbocyclyloxy,” as used herein, refers to a monovalent radical having the structure —O-carbocyclyl, e.g., a —O-cycloalkyl or a —O-cycloalkenyl radical. The terms “carbocyclyl,” “cycloalkyl,” and “cycloalkenyl” included in —O-carbocyclyl, —O-cycloalkyl, and —O-cycloalkenyl are as defined herein.

The term “cycloalkyl”, as used herein refers to a saturated carbocyclyl. Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spirocyclic compounds.

The term “cycloalkylene,” as used herein, refers to a divalent radical obtained by removing a hydrogen atom from a carbon atom of a cycloalkylene group. The divalency of a cycloalkylene group does not include the optional substituents on the cycloalkylene group

The term “cyano,” as used herein, refers to a monovalent radical having the structure —CN.

The term “cycloalkenyl,” as used herein, represents a monovalent, unsaturated carbocyclyl group that includes at least one carbon-carbon double bond, no carbon-carbon triple bond, only C and H when unsubstituted, and is not fully aromatic. A cycloalkenyl may have, e.g., four to fourteen carbons (e.g., a C₄-C₇, C₄-C₈, C₄-C₉, C₄-C₁₀, C₄-C₁₁, C₄-C₁₂, C₄-C₁₃, or C₄-C₁₄ cycloalkenyl). Exemplary cycloalkenyl groups include, but are not limited to, cyclopentenyl, cyclohexenyl, and cycloheptenyl. The term “cycloalkenyl” also includes cyclic groups having a bridged multicyclic structure in which one or more carbons bridges two non-adjacent members of a monocyclic ring, e.g., bicyclo[2.2.2]oct-2-ene. The term “cycloalkenyl” also includes fused ring bicyclic and multicyclic systems containing one or more double bonds, e.g., fluorene.

The term “cycloalkenylene,” as used herein, refers to a divalent radical obtained by removing a hydrogen atom from a carbon atom of a cycloalkenylene group. The divalency of a cycloalkenylene group does not include the optional substituents on the cycloalkenylene group

The term “halo,” as used herein, refers to a fluorine (fluoro), chlorine (chloro), bromine (bromo), or iodine (iodo) radical.

The term “heterocyclyl,” as used herein, represents a saturated or unsaturated monocyclic or fused ring bicyclic or polycyclic system having one or more carbon atoms and at least one heteroatom, e.g., one to four heteroatoms (e.g., one to four, one to three, one or two, one, two, three, or four heteroatoms), selected from N, O, and S. Heterocyclyl groups include both non-aromatic and aromatic systems. An aromatic heterocyclyl group is referred to as a “heteroaryl” group. In some embodiments, a heterocyclyl group is a 3- to 8-membered ring system, a 3- to 6-membered ring system, a 4- to 6-membered ring system, a 4- to 10-membered ring system, a 6- to 10-membered ring system, a 6- to 12-membered ring system, a 5-membered ring, or a 6-membered ring, or a ring or ring system having a number of ring atoms that fall within any of the above-mentioned ranges. Exemplary 5-membered heterocyclyl groups may have zero to two double bonds, and exemplary 6-membered heterocyclyl groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocyclyl groups include, but are not limited to, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocyclyl groups include, but are not limited to, optionally substituted azepine, optionally substituted 1,4-diazepine, optionally substituted thiepine, and optionally substituted 1,4-thiazepine. Exemplary 8- to 10-membered bicyclic groups include, but are not limited to, optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl.

The term “heterocyclylene,” as used herein, refers to a divalent radical obtained by removing a hydrogen atom from a ring atom of a heterocyclylene group. The divalency of a heterocyclylene group does not include the optional substituents on the heterocyclylene group. An aromatic heterocyclylene group is referred to as a “heteroarylene” group.

The term “N-protecting group,” as used herein, refers to a group protecting a nitrogen atom in a molecule from participating in one or more undesirable reactions during chemical synthesis (e.g., oxidation reactions, or certain nucleophilic and electrophilic substitutions). Commonly used N-protecting groups are disclosed in Wuts, Greene's Protective Groups in Organic Synthesis, Wiley-Interscience, 4th Edition, 2006. Exemplary N-protecting groups include acyl (e.g., formyl, acetyl, trifluoroacetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, and 4-bromobenzoyl); sulfonyl-containing groups (e.g., benzenesulfonyl, p-toluenesulfonyl, o-nitrobenzenesulfonyl, and p-nitrobenzenesulfonyl); carbamate forming groups (e.g., benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl), arylalkyl (e.g., triphenylmethyl); silyl groups (e.g., trimethylsilyl); and imine-forming groups (e.g., diphenylmethylene). Preferred N-protecting groups are acetyl, benzoyl, phenylsulfonyl, p-toluenesulfonyl, p-nitrobenzenesulfonyl, o-nitrobenzenesulfonyl, t-butyloxycarbonyl (Boc), and benzyloxycarbonyl (Cbz).

The term “oxo,” as used herein, refers to a divalent oxygen atom represented by the structure ═O.

The term “thioalkyl,” as used herein, refers to a monovalent radical having the structure —S-alkyl, in which “alkyl” is as defined herein.

The phrase “optionally substituted X,” as used herein, is intended to be equivalent to “X, wherein X is optionally substituted” (e.g., “alkyl, wherein said alkyl is optionally substituted”). It is not intended to mean that the feature “X” (e.g. alkyl) per se is optional. The term “optionally substituted,” as used herein, refers to having 0, 1, or more substituents (e.g., 0-10, 0-9, 0-8, 0-7, 0-6, 0-5, 0-4, 0-3, 0-2, 0 or 1, 0, 1, 2, 3, 4, 5, 6, 7, 8, 9, or 10 substituents).

Alkyl, alkylene, alkenyl, alkynyl, carbocyclyl, cycloalkyl, cycloalkenyl, aryl, heterocyclyl, and heterocyclylene groups may be substituted with one or more of carbocyclyl, cycloalkyl; cycloalkenyl; aryl; heterocyclyl; heteroaryl; halo; OH; cyano; alkoxy; alkenyloxy; thioalkyl; NO₂; N₃; NR^{b}R^{c}; wherein each of R^{b} and R^{c} is, independently, H, alkyl, alkenyl, alkynyl, cycloalkyl, cycloalkenyl, aryl, or heterocyclyl; SO₂R^{d}, wherein R^{d} is H, alkyl or aryl; SO₂NR^{e}R^{f}, wherein each of R^{e} and R^{f} is, independently, H, alkyl, or aryl; or NR^{b}SO₂R^{c}, wherein R^{b} and R^{c} are as defined above. Aryl, carbocyclyl, cycloalkyl, cycloalkenyl, heteroaryl, and heterocyclyl groups may also be substituted with alkyl, alkenyl, or alkynyl. Alkyl, alkoxy, carbocyclyl, cycloalkyl, cycloalkenyl, and unsaturated heterocyclyl groups may also be substituted with oxo. In some embodiments, a substituent is further substituted as described herein. For example, a C₆ aryl group, i.e., phenyl, may be substituted with an alkyl group, which may be further substituted with a heterocyclyl group.

The term “water miscible organic solvent,” as used herein, refers to an organic solvent that can form a homogenous mixture with water, and a weak base. Examples of such solvents include, but are not limited to, dimethyl sulfoxide, dimethylformamide, 1,2-propanediol, and other alcohol solvents such as methanol, ethanol, and 1,4-butanediol, or an alcohol solvent with a boiling point from about 90 to about 100° C. “Water miscible” means soluble in water up to 80%, 90%, 95%, or more organic solvent.

The term “weak base,” as used herein, refers to an organic or inorganic base of which the conjugate acid has a pKa of about 7 to about 12 in an aqueous solution. Exemplary inorganic weak bases include, but are not limited to, alkali metal carbonates (e.g., Na₂CO₃, K₂CO₃, Cs₂CO₃), alkali metal bicarbonates (e.g., NaHCO₃, KHCO₃), and alkali metal phosphates (e.g., Na₃PO₄, Na₂HPO₄, NaH₂PO₄, K₃PO₄, K₂HPO₄, KH₂PO₄). Exemplary organic weak bases include, but are not limited to, alkylamines (e.g., triethylamine, diethylamine, t-butylamine, n-butylamine, di-isopropylethylamine, and dimethylethylamine), pyridine, piperidine, morpholine, and DABCO.

### DETAILED DESCRIPTION OF THE DISCLOSURE

The present disclosure provides methods for the synthesis of small molecule of Factor D inhibitors and intermediates thereof. The small molecule inhibitors are compounds of formula (III):

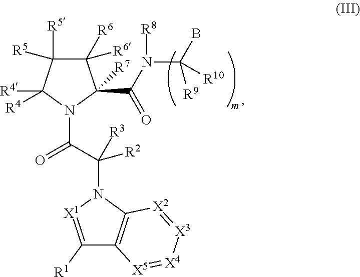

or pharmaceutically acceptable salt thereof. Exemplary compounds of formula (III) are described in, e.g., U.S. Pat. Nos. 9,796,741, 10,011,612, and 10,662,675 and U.S. Patent Publications Nos. 2019/0382376 A1, 2020/0002347 A1, and 2020/0071301 A1, the disclosures of which are incorporated herein by reference.

The method includes deprotecting a compound of formula (II) to form a compound of formula (I):

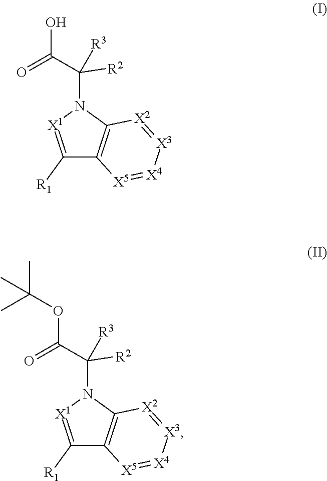

by reacting the compound of formula (II) with a weak base (e.g., potassium carbonate or cesium carbonate) in a water-miscible solvent (e.g., 1,2-propanediol), in which the variables in formulas (I), (II), and (Ill) are as defined above. The successful removal of the t-butyl group of the compounds of formula (I) is unexpected as it is well-known in the art that t-butyl esters are stable to mild basic hydrolysis and typically cleaved by moderately acidic hydrolysis (see, e.g., Chapter 5, pages 584-586 of Wuts, Greene's Protective Groups in Organic Synthesis, Wiley-Interscience, 4th Edition, 2006) or the use of a strong base such as KOH (See, e.g., E. Filali, et al., Synlett, 2009, 205-208). Indeed, prior to the present disclosure, deprotection of the compound for formula (II) was typically achieved by treating it with an acid, e.g., trifluoracetic acid in dichloromethane or methanesulfonic acid or sulfuric acid in a mixture of water and acetonitrile, and, in certain cases, strong bases such as NaOH in a mixture of tetrahydrofuran and water or LiOH in a mixture of methanol and water were used. Substituting the use of an acid or a strong base with deprotection with a weak base provides benefits including (i) the ability to use of safe, low-cost solvents, (ii) easier workup procedures, and/or (iii) higher yield and purity of the final product which may be less subject to degradation from weak bases compared to strong bases. These advantages provided by the methods described herein are beneficial to scaling up the synthetic process for preparing compounds of formula (III).

### Compounds of Formula (II)

Compounds of formula (II) can be prepared by first reacting a compound of formula (V):

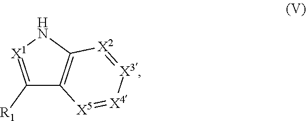

in which one of X³ and X^{4′} is N or CRₑ and the other is CBr, with potassium carbonate and a compound of formula (VI):

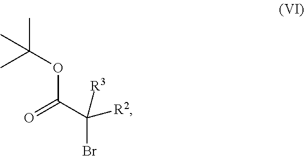

in refluxing acetonitrile to obtain a compound of formula (VII):

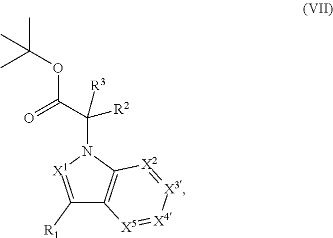

then reacting the compound of formula (VII) with an organoboron reagent containing the group Rr, such as a compound of formula (VIII):

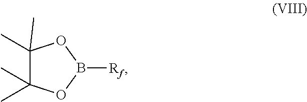

under Suzuki coupling reactions (e.g., in the presence of Pd(PPh₃)₄ and cesium carbonate in a 9:1 mixture of DMF and H₂O) to obtain a compound of formula (II). Suitable reagents for Suzuki coupling reactions (e.g., catalysts, solvents, and reagents such as organoboron reagents) are well-known in the art. Exemplary compounds of formula (II) are described in, e.g., U.S. Pat. Nos. 9,796,741, 10,011,612, and 10,662,675 and U.S. Patent Publications Nos. 2019/0382376 A1, 2020/0002347 A1, and 2020/0071301 A1, the disclosures of which are incorporated herein by reference.

### Compounds of Formula (III)

In some embodiments, the compound of formula (III) is prepared by coupling the compound of formula (I) to a compound of formula (IV):

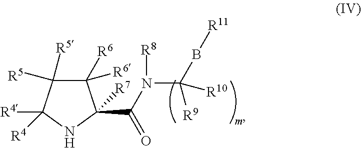

or a salt thereof, in which all variables are as defined for formula (III), under amidation reaction conditions.

In some embodiments, the hydrochloride salt of the compound of formula (IV) is coupled to the compound of formula (I). The reaction may be performed in dimethylformamide in the presence of 1-[bis(dimethylamino)methylene]-1H-1,2,3-triazolo[4,5-b]pyridinium 3-oxide hexafluorophosphate and N,N-diisopropylethylamine.

In some embodiments, the hydrobromide salt of the compound of formula (IV) is coupled to the compound of formula (I). The reaction may be performed in acetonitrile in the presence of propanephosphonic acid anhydride and N,N-diisopropylethylamine.

In some embodiments, the trifluoroacetic acid salt of the compound of formula (IV) is coupled to the compound of formula (I). The reaction may be performed in dimethylformamide in the presence of N,N-diisopropylethylamine and 1-[bis(dimethylamino)methylene]-1H-1,2,3-triazolo[4,5-b]pyridinium 3-oxide hexafluorophosphate or 2-(1H0benzotriazole-1-yl)-1,1,3,3-tetramethylaminium tetrafluoroborate.

### Compounds of Formula (IV)

The compound of formula (IV) can be prepared using the following reaction scheme:

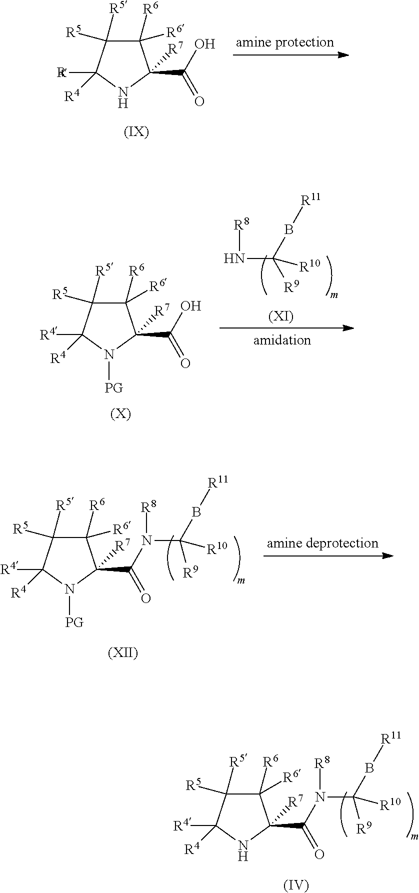

in which all variables of formulas (IX)—(XII) are as defined in formula (IV), and variable PG is an N-protecting group. Briefly, the compound of formula (IX) is treated with an amine protecting agent to form the compound of formula (X), which is then coupled to the compound of (XI) via an amidation reaction to form a compound of formula (XII). The compound of formula (IV) is then obtained from the compound of formula (XII) by deprotecting the amine group thereof (i.e., removing the N-protecting group).

In some embodiments, the amine protecting reagent is di-tert-butyl dicarbonate (Boc₂O), and the amine protection reaction is performed in an organic solvent (e.g., acetonitrile) in the presence of a base (e.g., 4-dimethylaminopyridine), and the N-protecting group is tert-butylcarbonate (Boc). In some embodiments in which the N-protecting group is Boc, the amine deprotection reaction includes treating the compound of formula (XII) with an acid in the presence of an organic solvent. In some embodiments, the acid is hydrochloric acid. In some embodiments, the organic solvent is dioxane. Other suitable amine protecting reagents and reaction conditions required to install and remove N-protecting groups are well known in the art (see, e.g., Wuts, Greene's Protective Groups in Organic Synthesis, Wiley-Interscience, 4th Edition, 2006).

In some embodiments, the amidation reaction is performed in an organic solvent in the presence of a base and a coupling reagent. In some embodiments, the organic solvent is dimethylformamide. In some embodiments, the base is diisopropylethylamine. In some embodiments, the coupling reagent is (1-[bis(dimethylamino)methylene]-1H-1,2,3-triazolo[4,5-b]pyridinium 3-oxide hexafluorophosphate (HATU).

Exemplary compounds of formula (IV) and their preparation methods are described in, e.g., U.S. Pat. Nos. 9,796,741, 10,011,612, and 10,662,675 and U.S. Patent Publications Nos. 2019/0382376 A1, 2020/0002347 A1, and 2020/0071301 A1, the disclosures of which are incorporated herein by reference.

### EXAMPLES

The examples described herein serve to illustrate the present invention, and the invention is not limited to the examples given.

### Comparative Example 1. Synthesis of 2-(3-acetyl-5-(2-methylpyrimidin-5-yl)-1H-indazol-1-yl)acetic acid

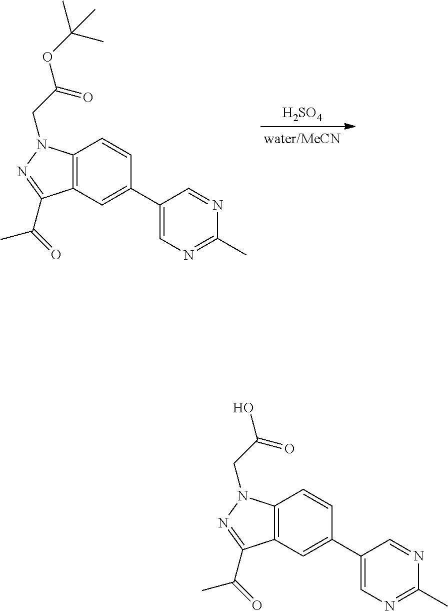 [SMILES: CC(=O)c1nn(CC(=O)OC(C)(C)C)c2ccc(-c3cnc(C)nc3)cc12 ; "H2SO4" ; CC(=O)c1nn(CC(=O)O)c2ccc(-c3cnc(C)nc3)cc12]

To a 250 mL three neck round bottom flask equipped with heating mantle, overhead stirring, thermocouple, and distillation head was charged tert-butyl 2-(3-acetyl-5-(2-methylpyrimidin-5-yl)-1H-indazol-1-yl)acetate (6 g, 16 mmol), acetonitrile (8 vol., 48 ml), and 2.6 wt. % sulfuric acid in water (10 vol., 60 mL). The mixture was heated to 75° C. until complete dissolution was achieved and then further heated to 82° C. to begin distillation. A total of 21 mL of distillate was removed and the condenser was switched from distillation to reflux. The mixture was held at 82° C. for an additional 3 h, during which in-process control (IPC) samples were pulled to monitor the reaction progress. After the reaction was complete, the mixture was cooled to 50° C. and additional water (24 mL) was added. The mixture was held at 50° C. for 30 min and then cooled to room temperature over 1.5 h. The mixture was held at room temperature for 1 h, filtered, and the solids was washed with water (30 mL) to afford 2-(3-acetyl-5-(2-methylpyrimidin-5-yl)-1H-indazol-1-yl)acetic acid (4.66 g, 91% yield) as a white solid. Water content was determined to be 0.09% by Karl Fischer titration. The compositions, as determined using ESI-MS, of the IPC samples, the filtered solids obtained after reaction completion, and the filtrate (or mother liquor; ML) are shown in the table below.

| | Dimer 1 | Dimer 2 | Product | SM |
|---|---|---|---|---|
| IPC 1 | ND | ND | 42.85 | 56.69 |
| IPC 2 | 1.55 | 1.37 | 96.18 | .76 |
| IPC 3 | 1.83 | 1.54 | 96.10 | .23 |
| Solids | ND | ND | 99.77 | .23 |
| ML | 18.83 | 16.51 | 63.01 | ND |

### Example 1. Synthesis of 2-(3-acetyl-5-(2-methylpyrimidin-5-yl)-1H-indazol-1-yl)acetic acid

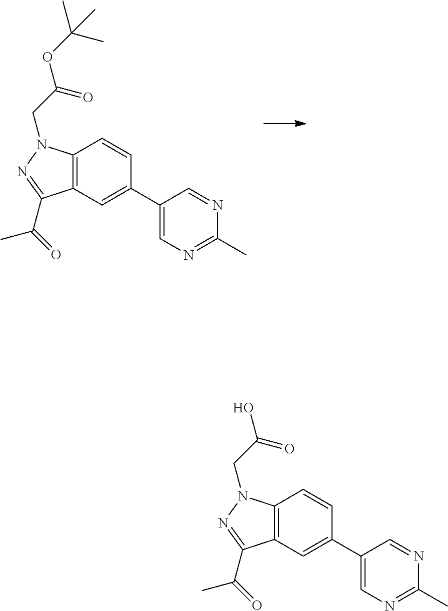 [SMILES: CC(=O)c1nn(CC(=O)OC(C)(C)C)c2ccc(-c3cnc(C)nc3)cc12 ; CC(=O)c1nn(CC(=O)O)c2ccc(-c3cnc(C)nc3)cc12]

### Experiment 1

A mixture of tert-butyl 2-(3-acetyl-5-(2-methylpyrimidin-5-yl)-1H-indazol-1-yl)acetate (0.5 g, 1.365 mmol) and potassium carbonate (0.566, 4.094 mmol) in a mixture of 1,2-propanediol (5 mL) and water (0.5 mL) was heated to approximately 90° C. for 1 hour. The reaction mixture was cooled to room temperature, and 6 N HCl(aq) was added until to pH 2. The resulting precipitate was collected and washed with water on a disposable polymer filter (Chemrus), then dried under vacuum with heating (˜30-50 mmHg at 40° C.) to afford 2-(3-acetyl-5-(2-methylpyrimidin-5-yl)-1H-indazol-1-yl)acetic acid (0.40 g, 1.290 mmol, 94.5% yield, 100% purity). The identity of the product and reaction completion were confirmed via ¹H NMR and HPLC/MS compared to a known sample. ¹H NMR (300 MHz, DMSO-d₆) δ 13.27 (bs, 1H), 9.04 (s, 2H), 8.43 (s, 1H), 7.94 (m, 1H), 7.88 (m, 1H) 5.50 (s, 2H), 2.66 (m, 6H).

The HPLC/MS analysis was carried out on an Acquity UPLC BEH C18 column (50 mm long×2.1 mm; 1.7 μm particle size) at room temperature under the following conditions:

| Time | Flow Rate | Water + 0.05% | Acetonitrile + 0.05% |
|---|---|---|---|
| (min) | (mL/min) | formic acid | formic acid |
| Initial | 0.800 | 85.0 | 15.0 |
| 0.24 | 0.800 | 85.0 | 15.0 |
| 3.50 | 0.800 | 15.0 | 85.0 |
| 4.00 | 0.800 | 15.0 | 85.0 |
| 4.50 | 0.800 | 85.0 | 15.0 |
| 4.80 | 0.800 | 85.0 | 15.0 |

### Experiment 2

A mixture of tert-butyl 2-(3-acetyl-5-(2-methylpyrimidin-5-yl)-1H-indazol-1-yl)acetate (1.0 g, 2.729 mmol) and potassium carbonate (1.132, 8.187 mmol) in a solvent mixture of n-propanol (10 mL) and water (2.0 mL) was heated under reflux overnight. The reaction mixture was cooled to room temperature, and 6 N HCl(aq) was added to about pH 2. The resulting precipitate was collected and washed with water on a disposable polymer filter (Chemrus), then dried under vacuum with heating (˜30-50 mmHg at 40° C.) to afford 2-(3-acetyl-5-(2-methylpyrimidin-5-yl)-1H-indazol-1-yl)acetic acid (0.83 g, 2.674 mmol, 98.0% yield, 100% purity). The identity of the product and reaction completion were confirmed via HPLC/MS compared to a known sample. The analysis was carried out on an Acquity UPLC BEH C18 column (50 mm long×2.1 mm; 1.7 μm particle size) at room temperature under the conditions set forth under Experiment 1 above.

### Example 2. 2-(3-acetyl-7-methyl-5-(2-methylpyrazolo[1,5-a]pyrimidin-6-yl)-1H-indol-1-yl)acetic acid

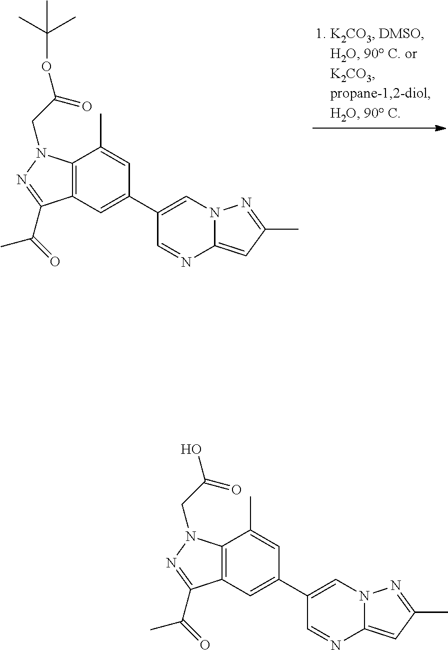

### Experiment 1

A mixture of tert-butyl 2-(3-acetyl-7-methyl-5-(2-methylpyrazolo[1,5-a]pyrimidin-6-yl)-1H-indol-1-yl)acetate (0.5 g, 1.195 mmol) and potassium carbonate (0.495, 3.584 mmol) in a mixture of DMSO (5 mL) and water (0.5 mL) was heated to approximately 90° C. for 4 hours. The reaction mixture was then cooled to room temperature, and 6 N HCl(aq) was added to about pH 2. The resulting precipitate was collected and washed with water on a disposable polymer filter (Chemrus), then dried under vacuum with heating (˜30-50 mmHg at 40° C.), to afford 2-(3-acetyl-7-methyl-5-(2-methylpyrazolo[1,5-a]pyrimidin-6-yl)-1H-indol-1-yl)acetic acid (0.41 g, 3.394 mmol, 94.7% yield, 100% purity). The identity of the product and reaction completion were confirmed via HPLC/MS compared to a known sample. The analysis was carried out on an Acquity UPLC BEH C18 column (50 mm long×2.1 mm; 1.7 μm particle size) at room temperature ° C. under the conditions set forth under Example 1, Experiment 1 above.

### Experiment 2

A mixture of tert-butyl 2-(3-acetyl-7-methyl-5-(2-methylpyrazolo[1,5-a]pyrimidin-6-yl)-1H-indol-1-yl)acetate (0.5 g, 1.195 mmol) and potassium carbonate (0.495, 3.584 mmol) in a mixture of 1,2-propanediol (5 mL) and water (0.5 mL) was heated to approximately 90° C. for 1 hour. The reaction mixture was then cooled to room temperature, and 6 N HCl(aq) was added to about pH 2. The resulting precipitate was collected and washed with water on a disposable polymer filter (Chemrus), then dried under vacuum with heating (˜30-50 mmHg at 40° C.) to afford 2-(3-acetyl-7-methyl-5-(2-methylpyrazolo[1,5-a]pyrimidin-6-yl)-1H-indol-1-yl)acetic acid (0.43 g, 3.559 mmol, 99.3% yield, 100% purity). The identity of the product and reaction completion were confirmed via HPLC/MS compared to a known sample. The analysis was carried out on an Acquity UPLC BEH C18 column (50 mm long×2.1 mm; 1.7 μm particle size) at room temperature under the conditions set forth under Example 1, Experiment 1 above.

### Example 3. Synthesis of 2-(3-acetyl-5-(2-methylpyrimidin-5-yl)-1H-indol-1-yl)acetic acid

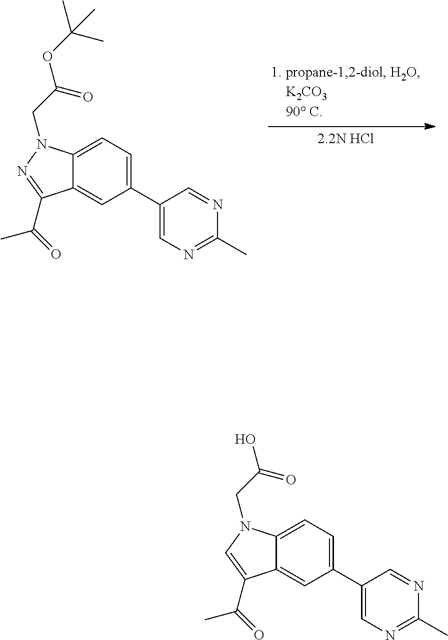 [SMILES: CC(=O)c1nn(CC(=O)OC(C)(C)C)c2ccc(-c3cnc(C)nc3)cc12 ; CC(=O)c1cn(CC(=O)O)c2ccc(-c3cnc(C)nc3)cc12]

To a 250 mL four-neck round bottom flask out fitted with a reflux condenser, stir bar, thermocouple, and a gas inlet adapter was charged 2.8 g of the tert-butyl 2-(3-acetyl-5-(2-methylpyrimidin-5-yl)-1H-indol-1-yl)acetate (7.4 mmol), followed by 3.1 g (22.4 mmol) potassium carbonate, 28 mL of propane-1,2-diol, and 2.8 mL water. The slurry was heated to an internal temperature between 85-90° C. and dissolution was observed. The reaction was held for 1 hr. IPC showed less than 1% starting material. The reaction was cooled to 25° C. and the pH was adjusted by the slow addition of 2 N HCl to a final pH of 2-2.5. The solids were then collected by filtration and dried at 50° C. in a vacuum oven overnight to obtain the title compound (2.47 g, 6.86 mmol, 93% yield, 100% purity as determined by reverse phase HPLC with gradient elution). ¹H NMR (300 MHz, DMSO-d₆) δ 13.39 (bs, 1H), 8.97 (s, 2H), 8.37 (m, 2H), 7.38 (s, 1H), 5.31 (s, 2H), 3.37-2.45 (m, 9H).

### Other Embodiments

Various modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of preparing a compound of formula (I), comprising reacting a compound of formula (II) with a weak base in a water-miscible organic solvent: 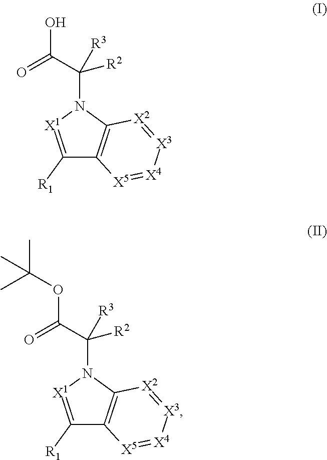
wherein
R¹ is H; halo; OH; NH₂; cyano; optionally substituted C₁-C₆ alkyl; optionally substituted C₂-C₆ alkenyl; optionally substituted 3- to 8-membered heterocyclyl; —C(O)NRₐRₐ′, wherein each of Rₐ and Rₐ′ is, independently, H, optionally substituted C₁-C₆ alkyl, optionally substituted C₂-C₆ alkenyl, optionally substituted C₂-C₆ alkynyl, or optionally substituted C₃-C₈ cycloalkyl; —C(O)R_{b}; —OC(O)R_{b}; or —C(O)OR_{b}; wherein R_{b}, in each instance, is selected from H, optionally substituted C₁-C₆ alkyl, optionally substituted C₁-C₆ alkoxy, and optionally substituted C₃-C₈ carbocyclyl;
each of R² and R³ is, independently, H or optionally substituted C₁-C₆ alkyl;
X¹ is N or CR_{c}, wherein R_{c} is H, halo, optionally substituted C₁-C₆ alkyl, or optionally substituted C₁-C₆ alkoxy;
each of X² and X⁵ is independently N or CR_{d}, wherein each R_{d} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and
each of X³ and X⁴ is independently selected from N, CRₑ, and CR_{f}, wherein Rₑ is selected from H, halo, cyano, optionally substituted C₁-C₆ alkyl, optionally substituted C₁-C₆ alkoxy, and —C(O)OR_{g}, wherein R_{g} is H or optionally substituted C₁-C₆ alkyl; and R_{f} is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S; wherein at least one of X³ and X⁴ is CR_{f}.

2. The method of claim 1, further comprising preparing a compound of formula (III): 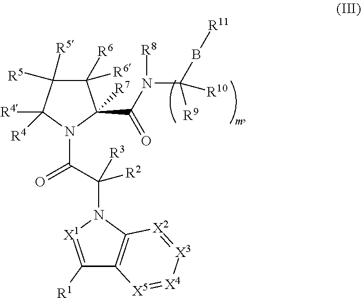
or a pharmaceutically acceptable salt thereof from the compound of formula (I),
wherein
each of R⁴, R^{4′}, R⁵, R^{5′}, R⁶, R^{6′}, and R⁷ is, independently, H; cyano; halo; OH; nitro; optionally substituted C₁-C₆ alkyl; optionally substituted C₂-C₆ alkenyl; C₁-C₆ alkoxy; C₁-C₆ thioalkyl; optionally substituted C₃-C₈ carbocyclyl; optionally substituted C₃-C₈ carbocyclyloxy; —NR_{g}R_{g}′; —C(O)NR_{g}R_{g}′; —OC(O)NR_{g}R_{g}′; —NR_{g}C(O)Rₕ; —NR_{g}C(O)ORₕ; —C(O)Rₕ; —C(O)ORₕ; or —C═NRₕ, wherein each of R_{g}, R_{g}′, and R^{h}, in each instance, is independently selected from H, optionally substituted C₁-C₆ alkyl, optionally substituted C₂-C₆ alkenyl, optionally substituted C₂-C₆ alkynyl, and optionally substituted C₃-C₈ carbocyclyl; or
R⁴ and R⁵, together with the atoms to which each is attached, form an optionally substituted C₃-C₆ cycloalkyl; or
R⁵ and R⁶, together with the atoms to which each is attached, form an optionally substituted C₃-C₆ cycloalkyl; or
R⁴ and R⁶ or R⁵ and R⁷ combine to form an optionally substituted C₁-C₂ alkylene; or
R⁴ and R^{4′}, R⁵ and R^{5′}, or R⁶ and R^{6′} combine to form oxo;
R⁸ is H or optionally substituted C₁-C₆ alkyl;
each of R⁹ and R¹⁰ is, independently, H or methyl;
R¹¹ is H; or
R⁵ and R¹¹ combine to form a group of formula —Y¹-Y²-Y³—;
each of Y¹ and Y² is selected from optionally substituted methylene; optionally substituted ethylene; —CH₂O—; —CH₂NRᵢ; —CH₂NRᵢC(O)—; —CH₂NRᵢS(O)₂—; —CH₂S(O)₂NRᵢ—; —CH₂(4- to 6-membered heterocyclylene)-; —CH₂O(4- to 6-membered heterocyclylene)-, wherein Rᵢ, in each instance, is H or optionally substituted C₁-C₆ alkyl;
Y³ is optionally substituted C₁-C₆ alkylene or optionally substituted C₂-C₆ alkenylene;
m is 0, 1, or 2;
B is optionally substituted C₁-C₆ alkylene, optionally substituted C₂-C₆ alkenylene, optionally substituted C₃-C₁₀ carbocyclylene, optionally substituted C₆-C₁₄ arylene, or optionally substituted 5- to 10-membered heterocyclylene; and
all other variables are as defined for formula (I) above.

3. The method of claim 2, wherein said preparing the compound of formula (III) or the pharmaceutically acceptable salt thereof comprises coupling the compound for formula (I) to the compound of formula (IV): 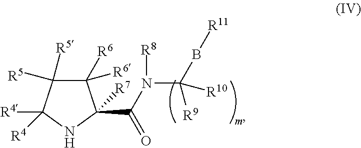
or a salt thereof, wherein all variables are as defined for formula (III).

4. The method of claim 3, wherein said preparing the compound of formula (III) or the pharmaceutically acceptable salt thereof comprises coupling the compound of formula (I) to the hydrochloride salt of the compound of formula (IV).

5. The method of claim 4, wherein said coupling the compound of formula (I) to the hydrochloride salt of the compound of formula (IV) occurs in dimethylformamide in the presence of 1-[bis(dimethylamino)methylene]-1H-1,2,3-triazolo[4,5-b]pyridinium 3-oxide hexafluorophosphate and N,N-diisopropylethylamine.

6. The method of claim 3, wherein said preparing the compound of formula (III) or the pharmaceutically acceptable salt thereof comprises coupling the compound of formula (I) to the hydrobromide salt of the compound of formula (IV).

7. The method of claim 6, wherein said coupling the compound of formula (I) to the hydrobromide salt of the compound of formula (IV) occurs in acetonitrile in the presence of propanephosphonic acid anhydride and N,N-diisopropylethylamine.

8. The method of claim 3, wherein said preparing the compound of formula (III) or the pharmaceutically acceptable salt thereof comprises coupling the compound of formula (I) to the trifluoroacetic acid salt of the compound of formula (IV).

9. The method of claim 8, wherein said coupling the compound of formula (I) to the trifluoroacetic acid salt of the compound of formula (IV) occurs in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

10. The method of any one of claims 2-9, wherein R⁸ is H.

11. The method of any one of claims 2-9, wherein R⁸ is CH₃.

12. The method of any one of claims 2-11, wherein m is 1.

13. The method of any one of claims 2-11, wherein m is 2.

14. The method of any one of claims 2-11, wherein m is 0.

15. The method of any one of claims 2-13, wherein each of R⁹ and R¹⁰ is H.

16. The method of any one of claims 2-13, wherein R⁹ is H and R¹⁰ is CH₃.

17. The method of any one of claims 2-13, wherein each of R⁹ and R¹⁰ is CH₃.

18. The method of any one of claims 2-17, wherein R⁵ is fluoro.

19. The method of claim 18, wherein each of R⁴, R^{4′}, R⁶, R^{6′}, and R⁷ is hydrogen.

20. The method of claim 19, wherein

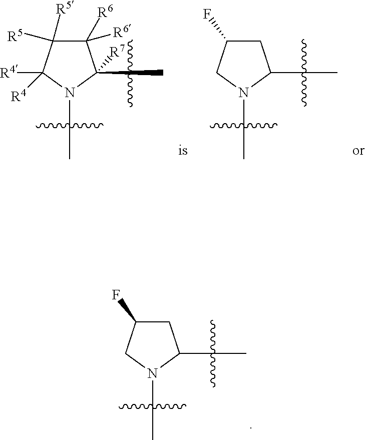

21. The method of claim 20, wherein

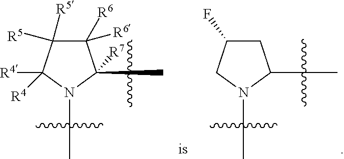

22. The method of claim 19, wherein R^{5′} is optionally substituted C₁-C₆ alkyl.

23. The method of claim 22, wherein

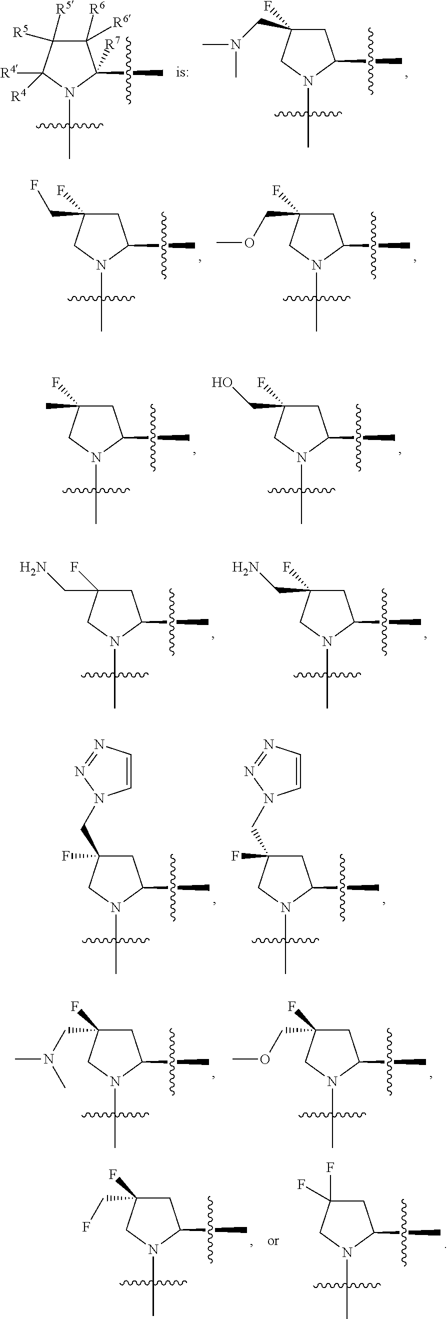

24. The method of claim 18, wherein

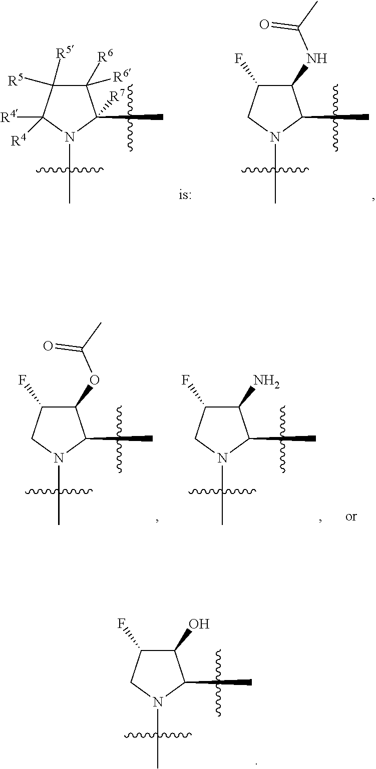

25. The method of any one of claims 2-17, wherein R⁴ and R⁵, together with the atoms to which each is attached, form an optionally substituted C₃-C₆ cycloalkyl; and each of R^{4′}, R⁶, R^{6′}, and R⁷ is H.

26. The method of claim 25, wherein R⁴ and R⁵, together with the atoms to which each is attached, form an optionally substituted cyclopropyl.

27. The method of claim 25 or 26, wherein R^{5′} is H.

28. The method of claim 27, wherein

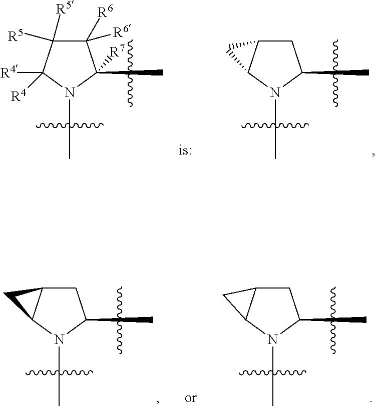

29. The method of claim 25 or 26, wherein R^{5′} is optionally substituted C₁-C₆ alkyl.

30. The method of claim 29, wherein

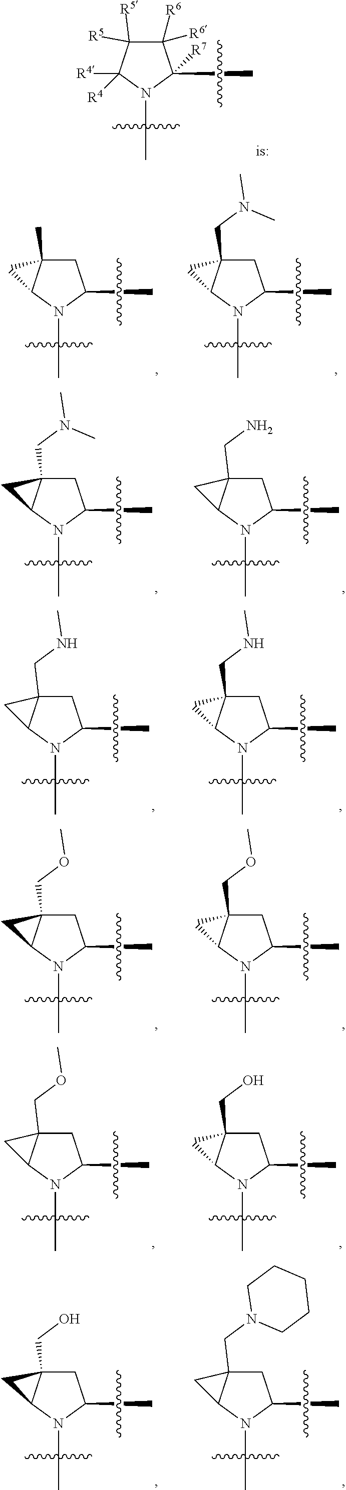

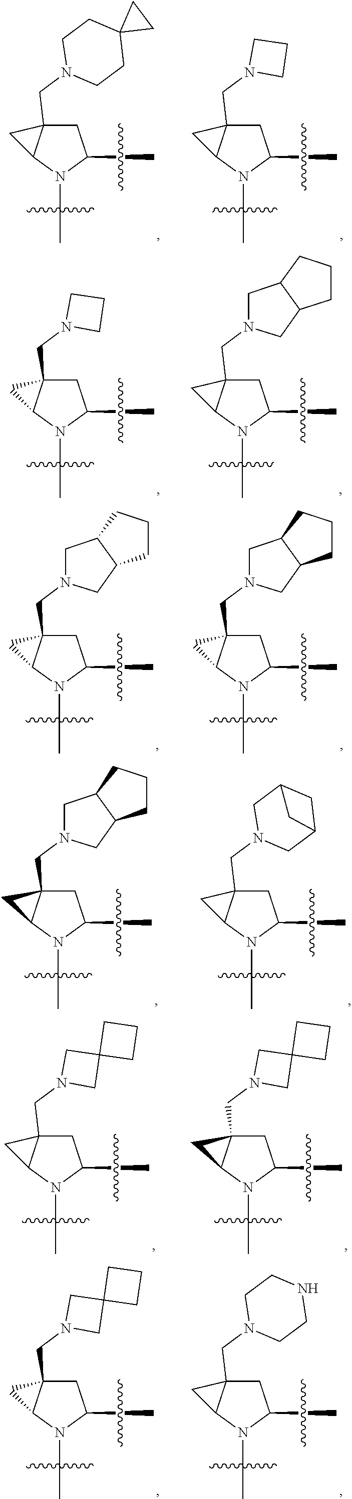

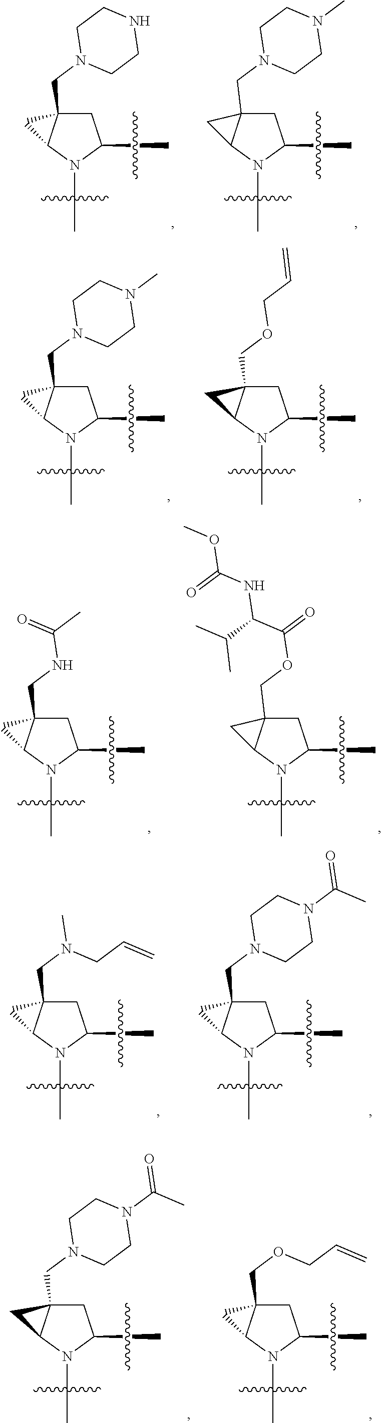

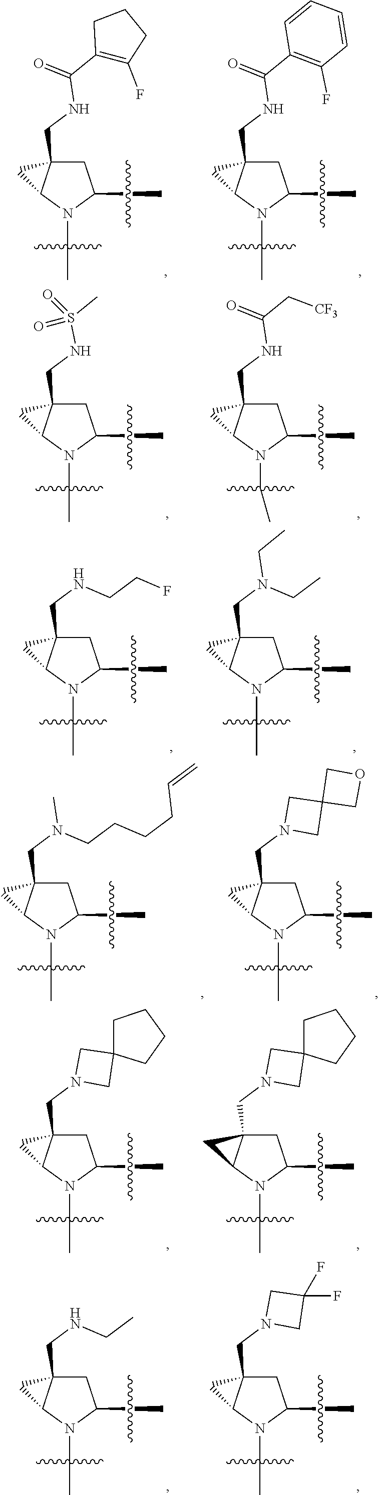

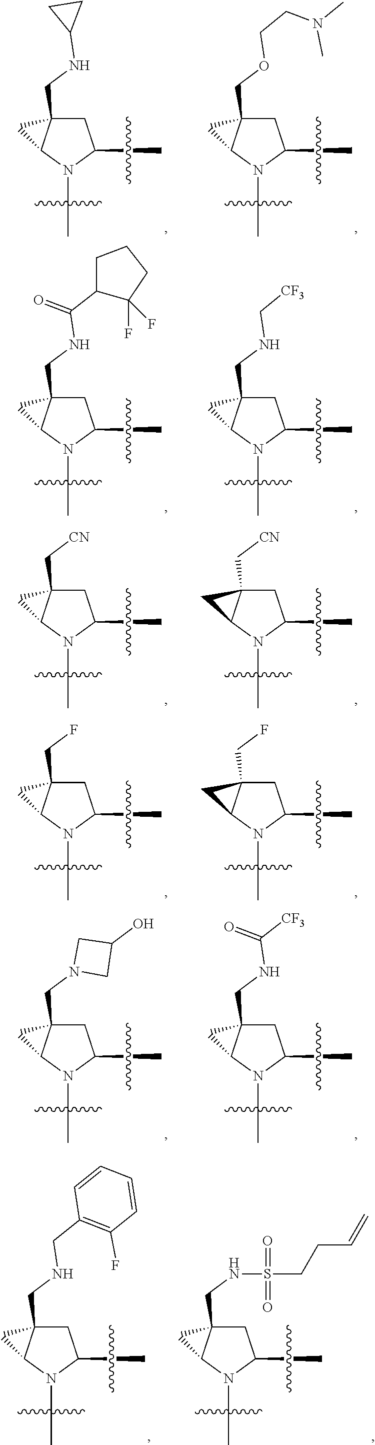

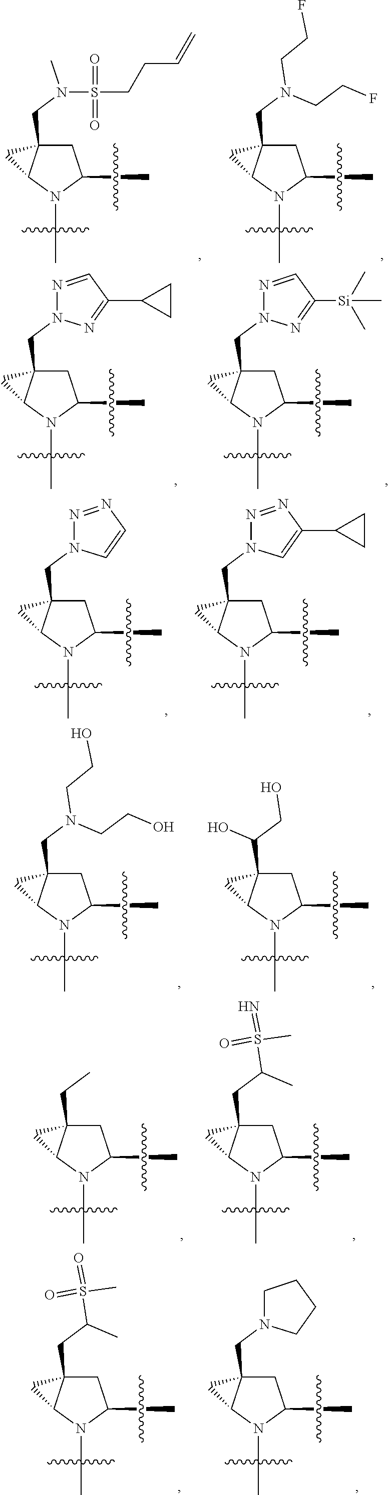

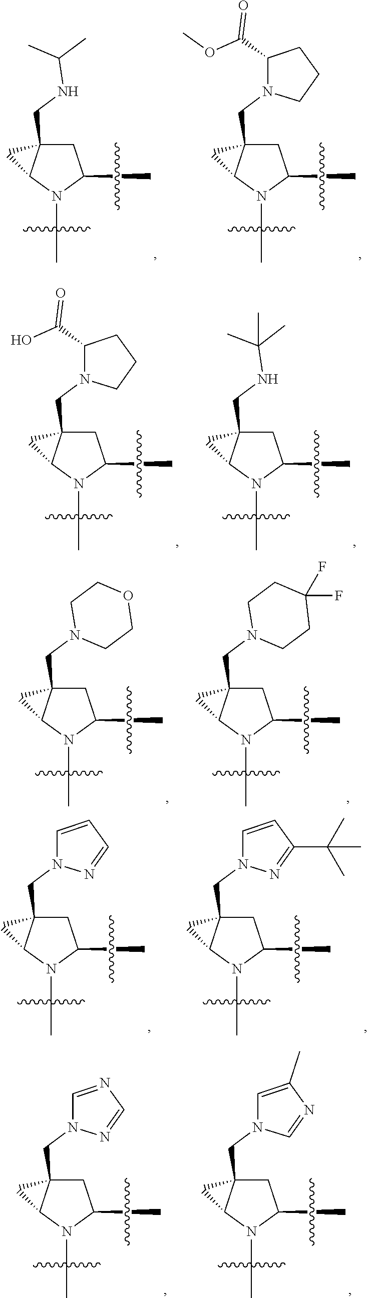

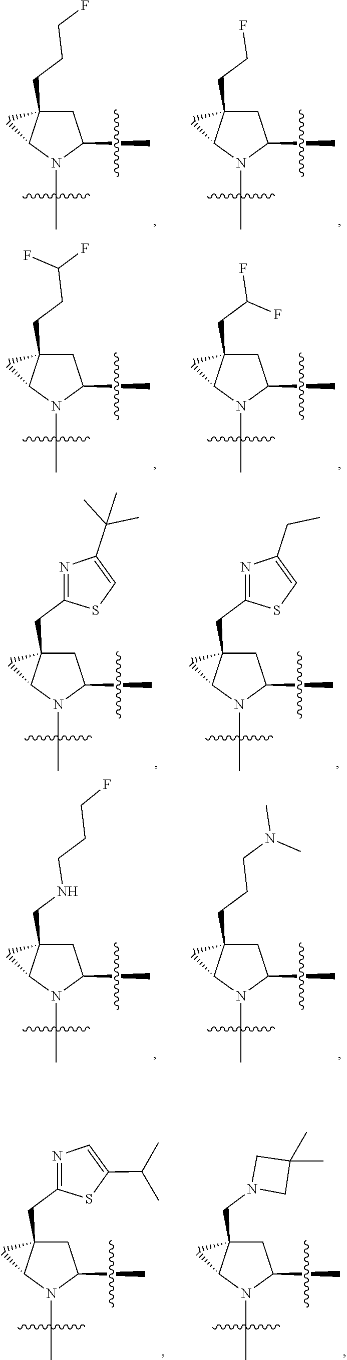

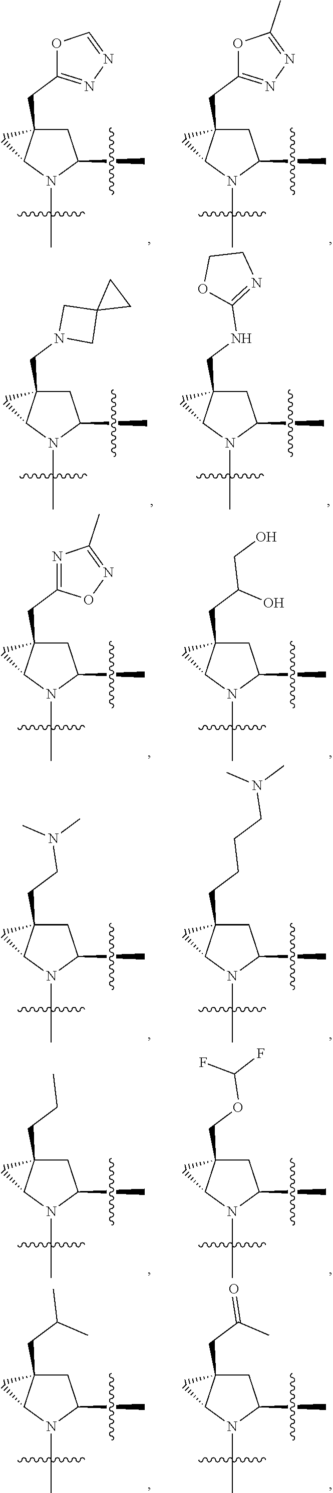

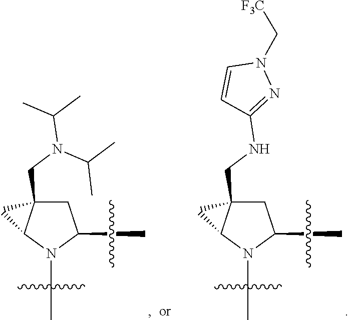

31. The method of claim 29 or 30, wherein

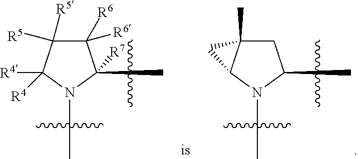

32. The method of claim 25 or 26, wherein

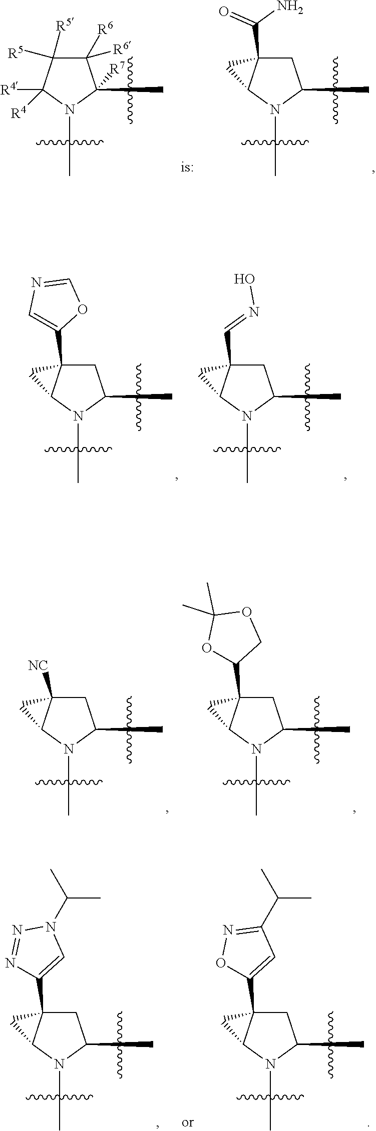

33. The method of any one of claims 2-17, wherein R⁵ and R⁶, together with the atoms to which each is attached, form an optionally substituted C₃-C₆ cycloalkyl, and each of R⁴, R^{4′}, R^{5′}, R^{6′}, and R⁷ is H.

34. The method of claim 33, wherein R⁵ and R⁶, together with the atoms to which each is attached, form an optionally substituted cyclopropyl.

35. The method of claim 34, wherein

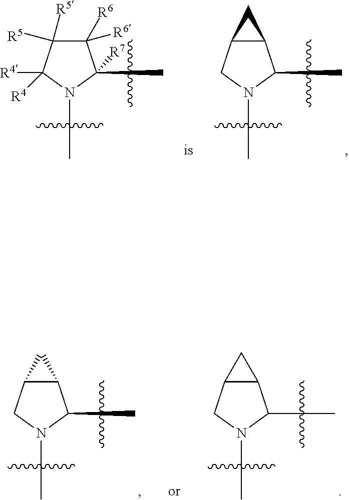

36. The method of any one of claims 2-17, wherein R⁵ and R⁷ combine to form optionally substituted C₁-C₂ alkylene.

37. The method of claim 36, wherein

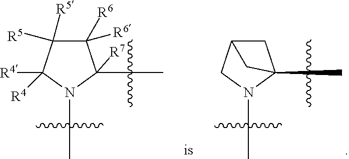

38. The method of any one of claims 2-17, wherein

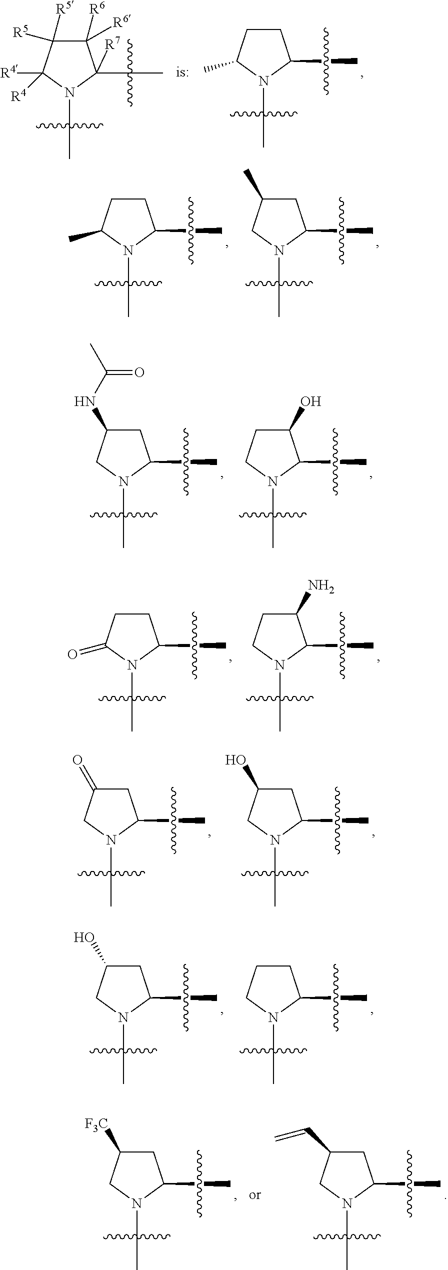

39. The method of any one of claims 2-38, wherein BR¹¹ is optionally substituted 5- to 10-membered heteroaryl.

40. The method of claim 39, wherein BR¹¹ is optionally substituted 6-membered heteroaryl.

41. The method of claim 40, wherein BR¹¹ is optionally substituted pyridyl, optionally substituted pyridazinyl, optionally substituted pyrimidinyl, or optionally substituted pyrazinyl.

42. The method of claim 41, wherein BR¹¹ is optionally substituted pyridyl.

43. The method of any one of claims 39-42, wherein BR¹¹ is:

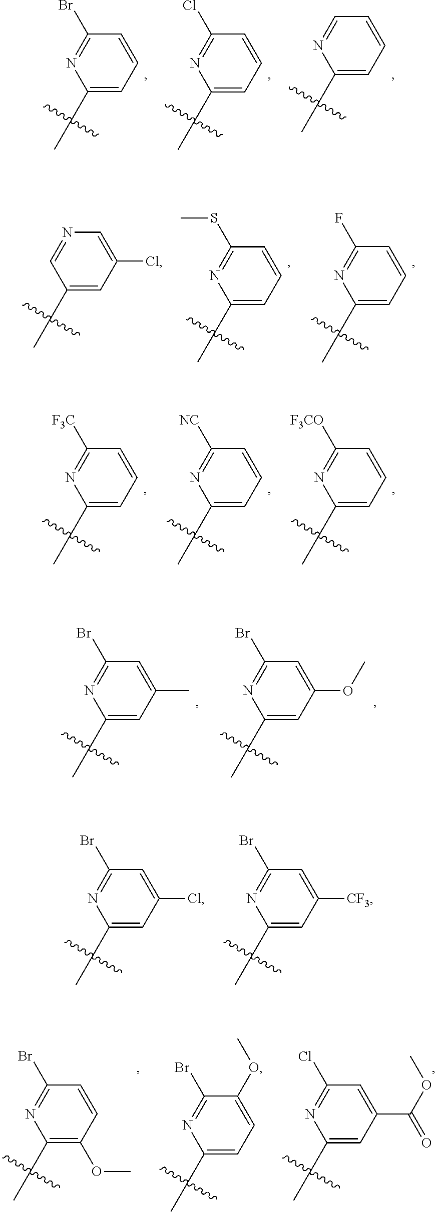

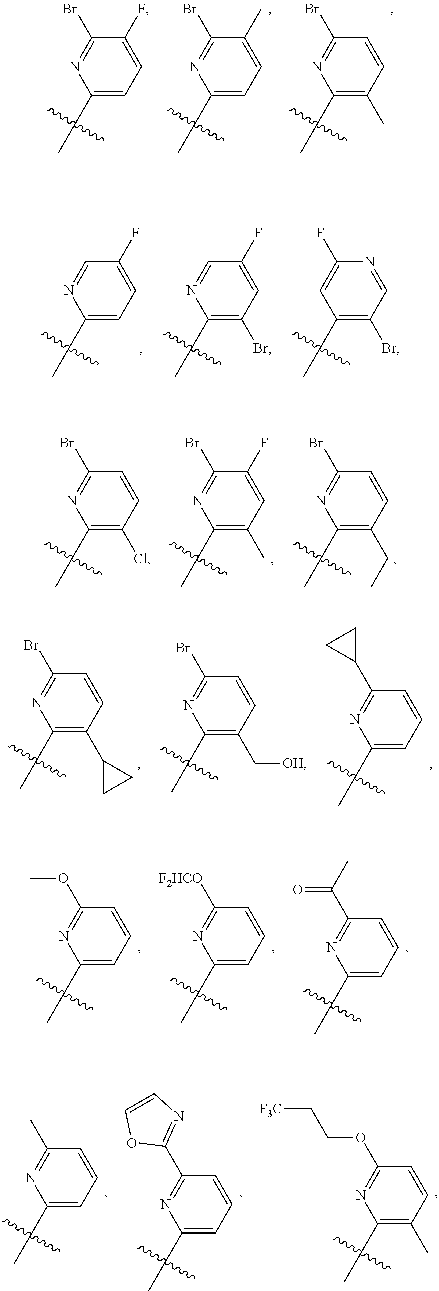

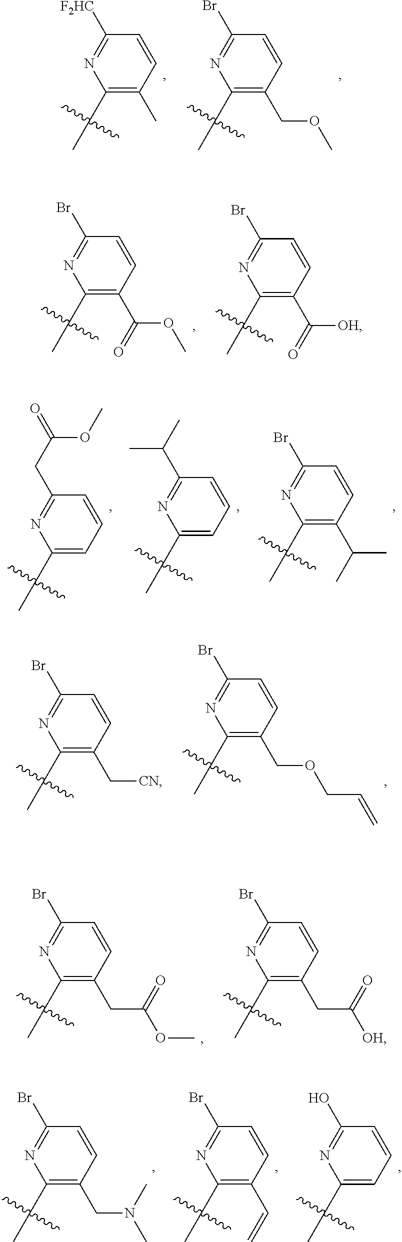

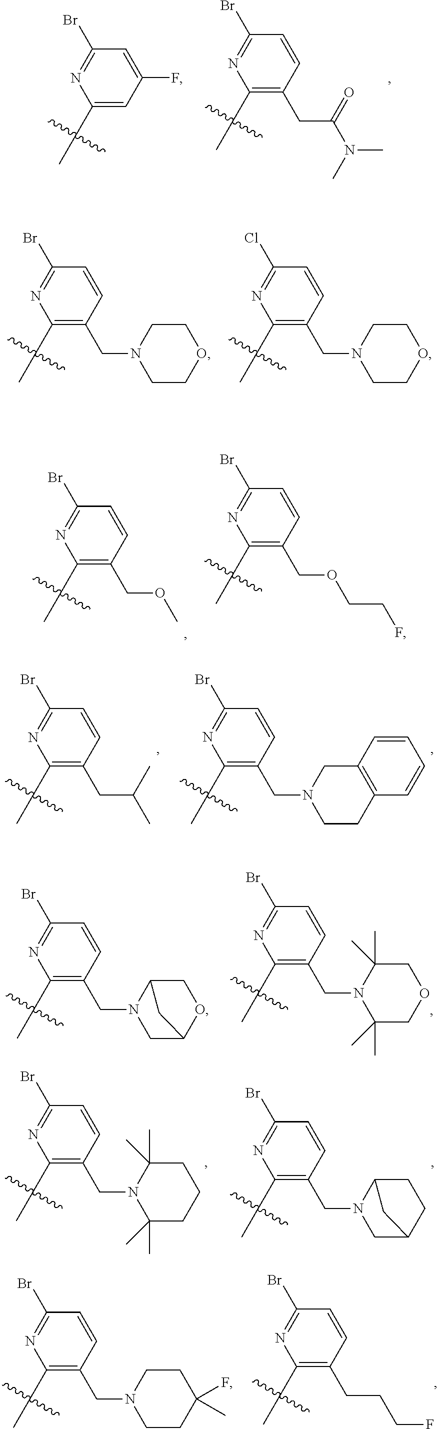

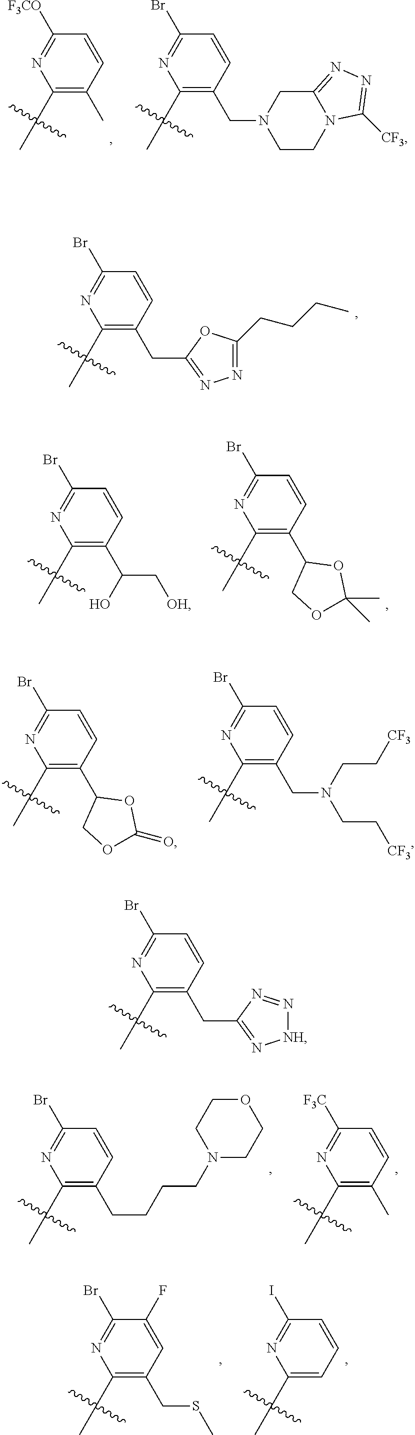

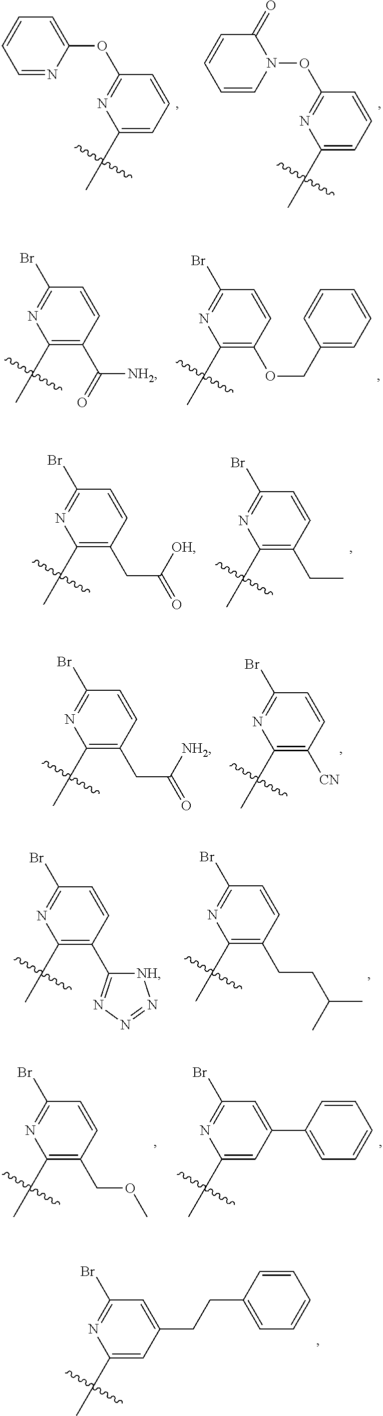

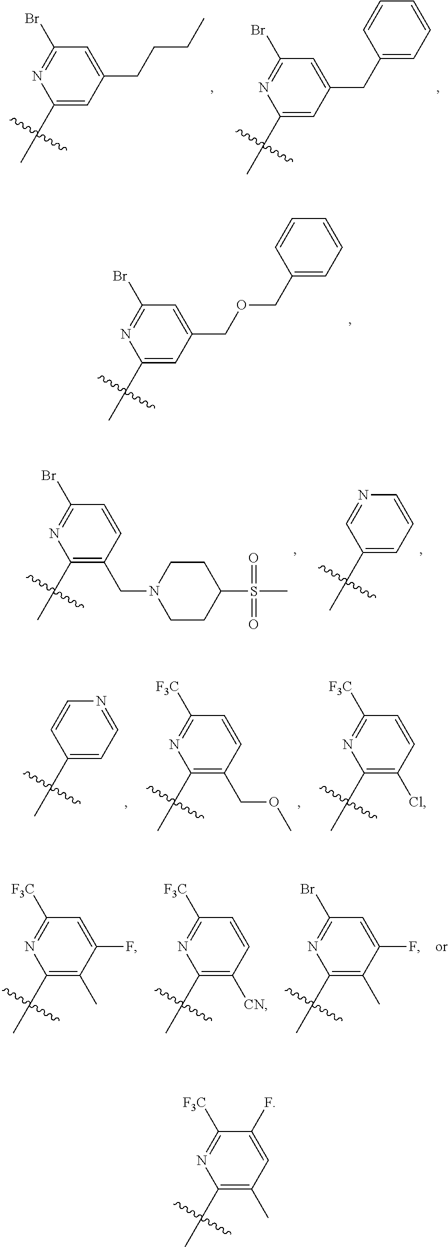

44. The method of claim 43, wherein BR¹¹ is

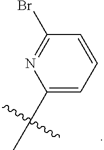 [SMILES: *c1cccc(Br)n1]

45. The method of claim 43, wherein BR¹¹ is

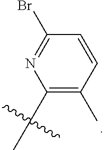 [SMILES: *c1nc(Br)ccc1C]

46. The method of claim 41, wherein BR¹¹ is optionally substituted pyrazinyl.

47. The method of claim 46, wherein BR¹¹ is:

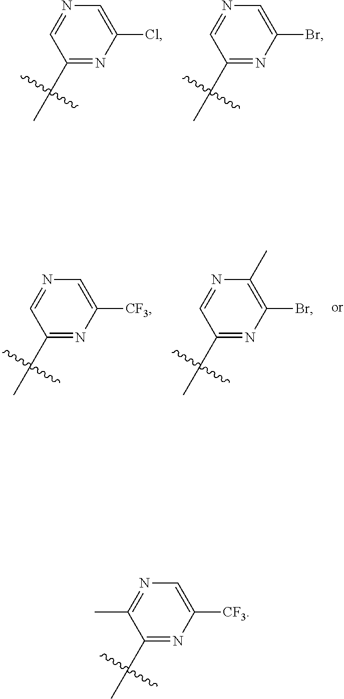

48. The method of claim 41, wherein BR¹¹ is optionally substituted pyrimidinyl.

49. The method of claim 48, wherein BR¹¹ is:

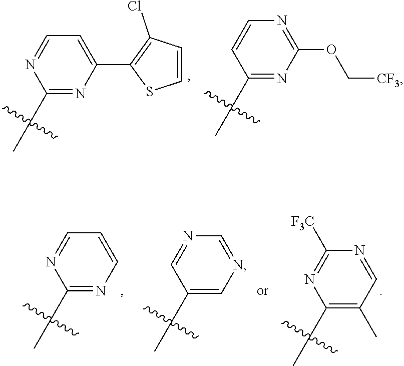 [SMILES: *c1nccc(-c2sccc2Cl)n1 ; *c1ccnc(OCC(F)(F)F)n1 ; *c1ncccn1 ; *c1cncnc1 ; *c1nc(C(F)(F)F)ncc1C]

50. The method of claim 41, wherein BR¹¹ is optionally substituted pyridazinyl.

51. The method of claim 50, wherein BR¹¹ is

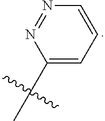 [SMILES: *c1cccnn1]

52. The method of claim 39, wherein BR¹¹ is optionally substituted five-membered heteroaryl.

53. The method of claim 52, wherein BR¹¹ is:

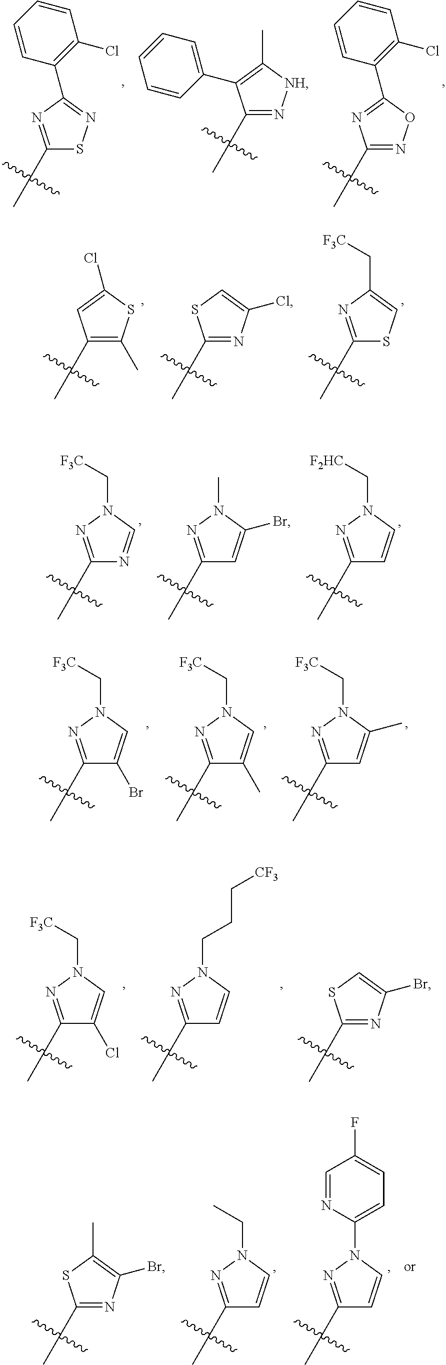

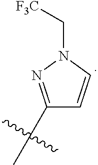 [SMILES: *c1ccn(CC(F)(F)F)n1]

54. The method of claim 39, wherein BR¹¹ is bicyclic 9- or 10-membered bicyclic heteroaryl.

55. The method of claim 54, wherein BR¹¹ is

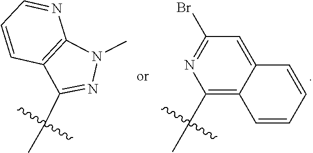 [SMILES: *c1nn(C)c2ncccc12 ; *c1nc(Br)cc2ccccc12]

56. The method of any one of claims 2-38, wherein BR¹¹ is optionally substituted C₆-C₁₄ aryl.

57. The method of claim 56, wherein BR¹¹ is optionally substituted phenyl.

58. The method of claim 57, wherein BR¹¹ is:

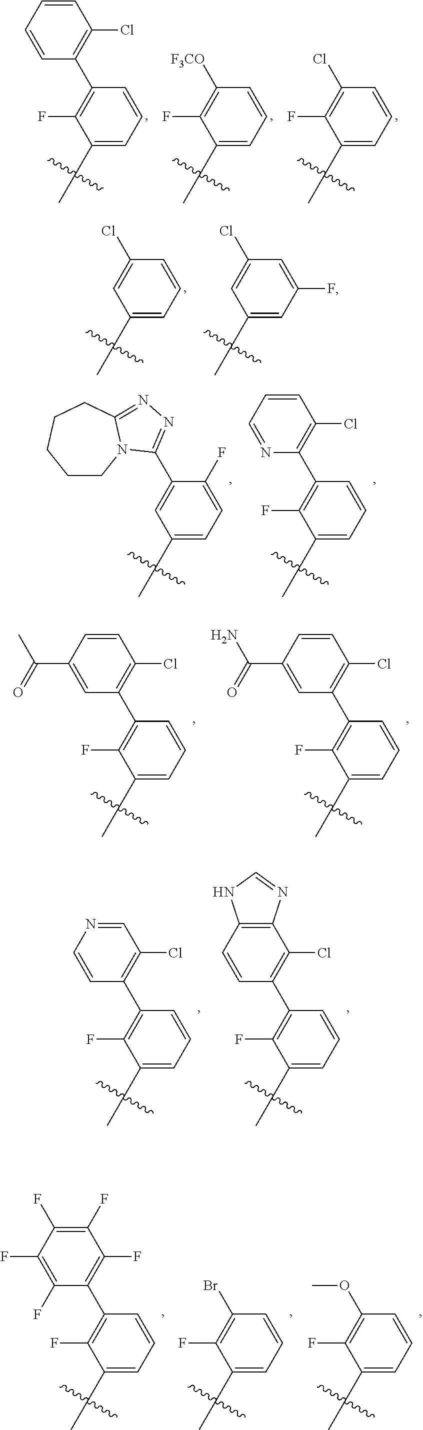

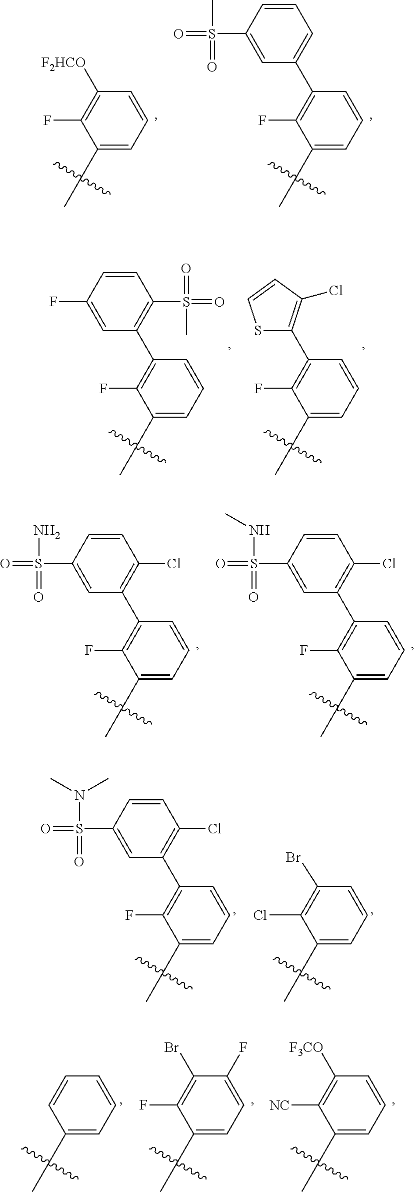

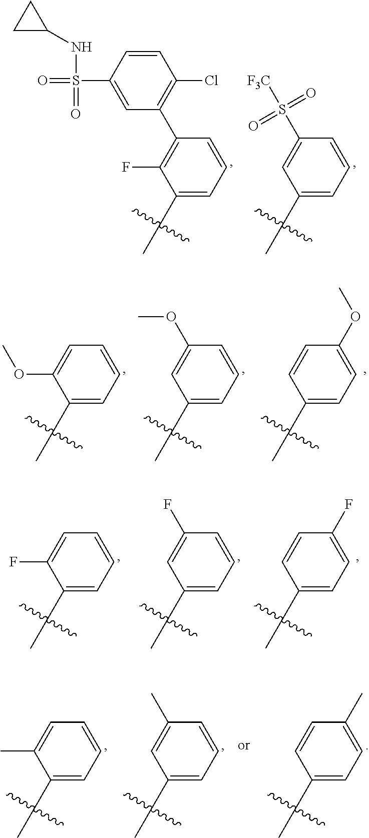

59. The method of any one of claims 2-38, wherein BR¹¹ is optionally substituted 5- to 9-membered unsaturated heterocyclyl.

60. The method of claim 59, wherein BR¹¹ is:

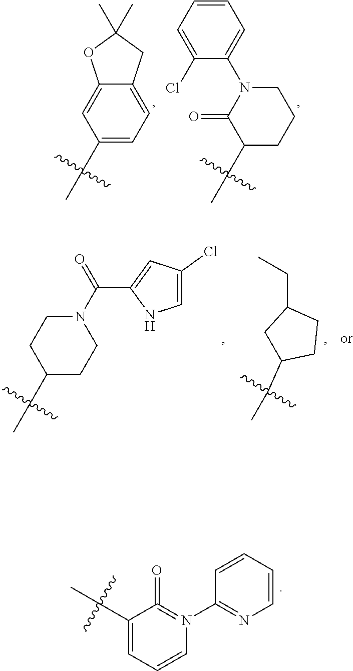

61. The method of any one of claims 2-38, wherein BR¹¹ is optionally substituted C₃-C₁₀ cycloalkyl.

62. The method of claim 61, wherein BR¹¹ is:

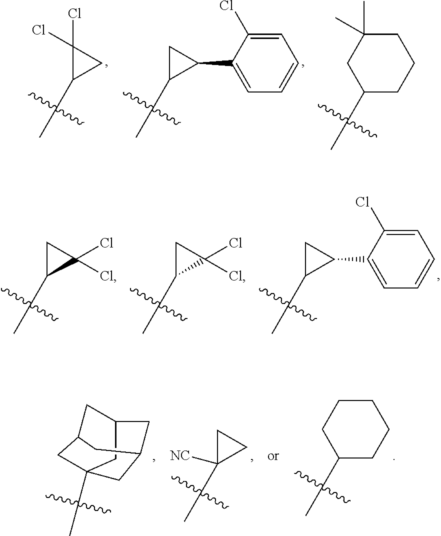

63. The method of any one of claims 2-38, wherein BR¹¹ is optionally substituted C₂-C₆ alkenyl.

64. The method of claim 63, wherein BR¹¹ is:

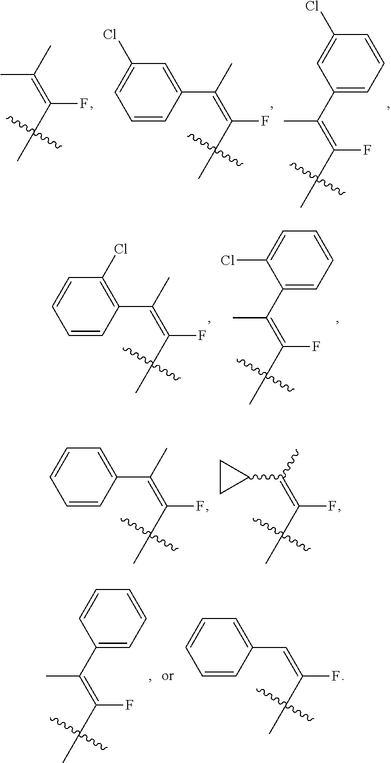

65. The method of any one of claims 2-38, wherein BR¹¹ is optionally substituted C₁-C₆ alkyl.

66. The method of claim 65, wherein BR¹¹ is:

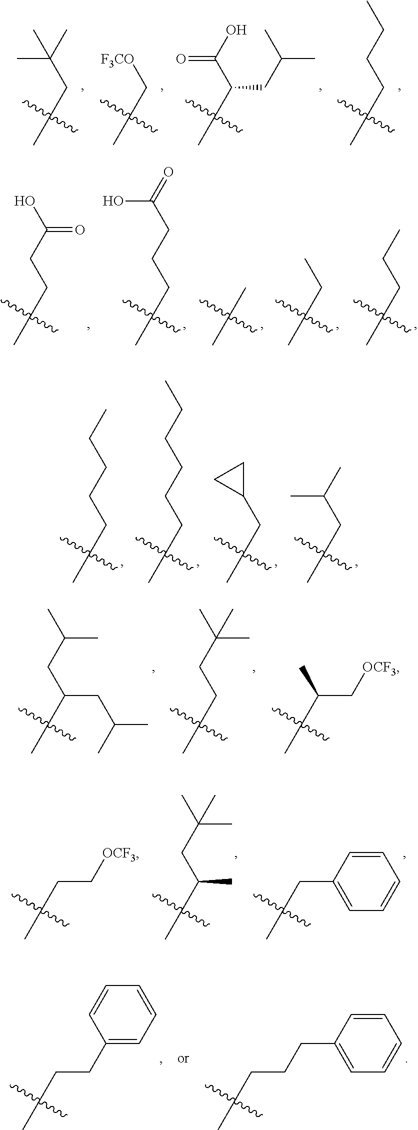

67. The method of any one of claims 2-17, wherein R⁵ and R¹¹ combine to form a group of formula —Y¹-Y²-Y³—.

68. The method of claim 67, wherein

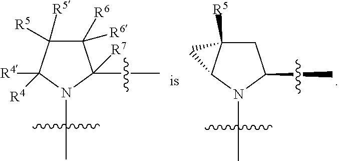

69. The method of claim 67, wherein is

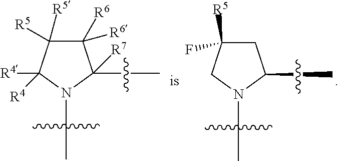

70. The method of any one of claims 67-69, wherein BR¹¹ is

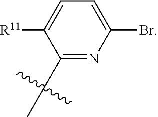

71. The method of any one of claims 69-69, wherein BR¹¹ is

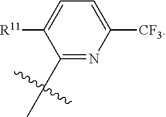

72. The method of any one of claims 67-71, wherein —Y¹-Y²-Y³— is:

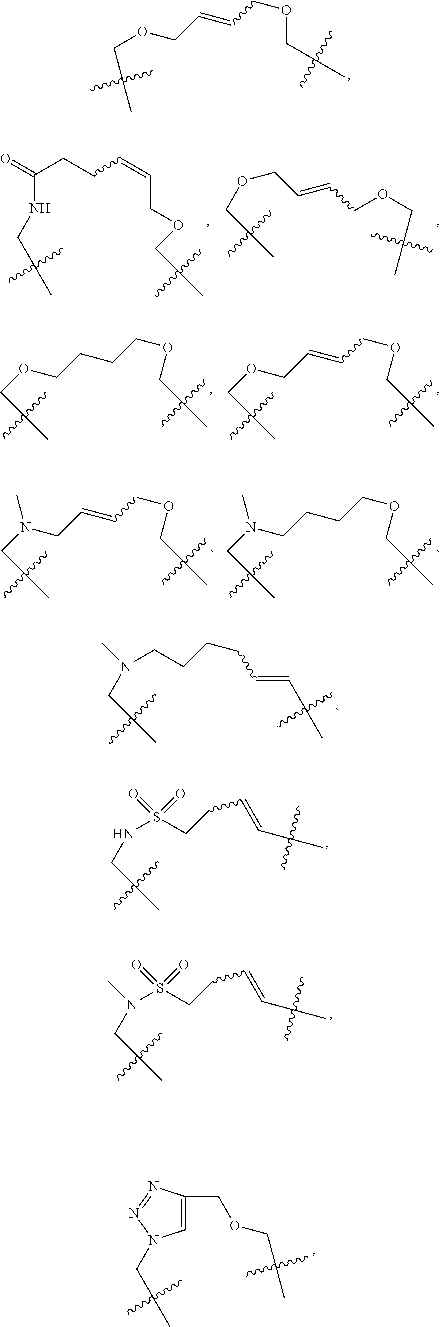

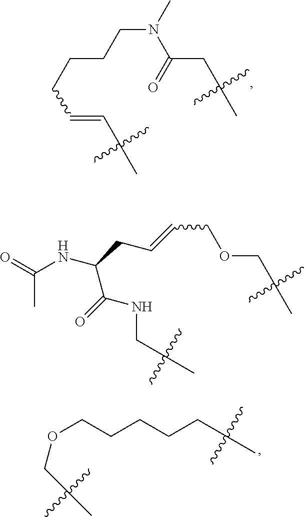 [SMILES: *C=CCCCCN(C)C(=O)C* ; *CNC(=O)[C@H](CC=CCOC*)NC(C)=O ; *CCCCCOC*]

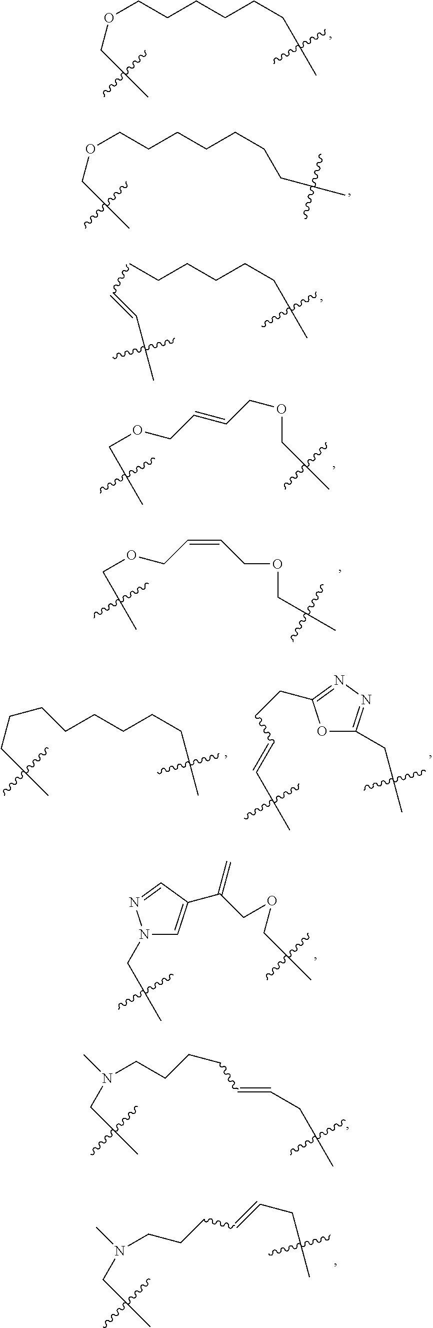

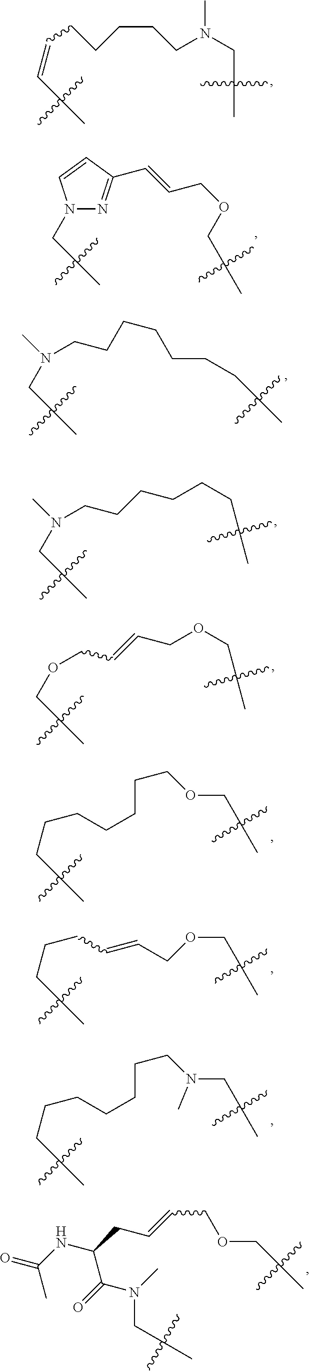

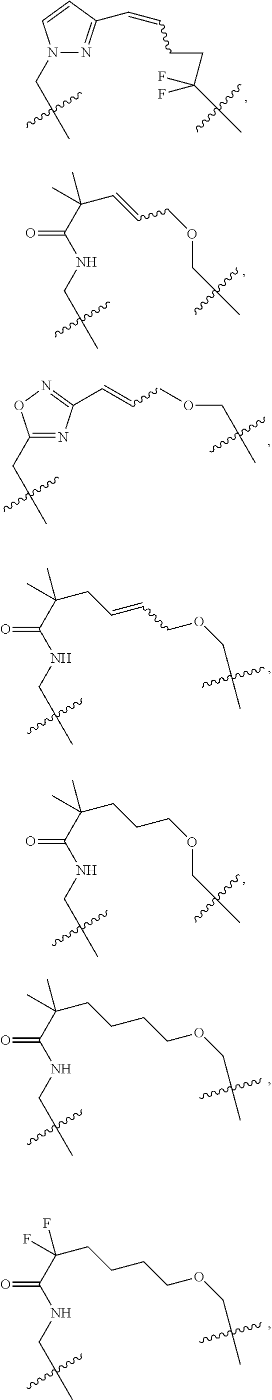

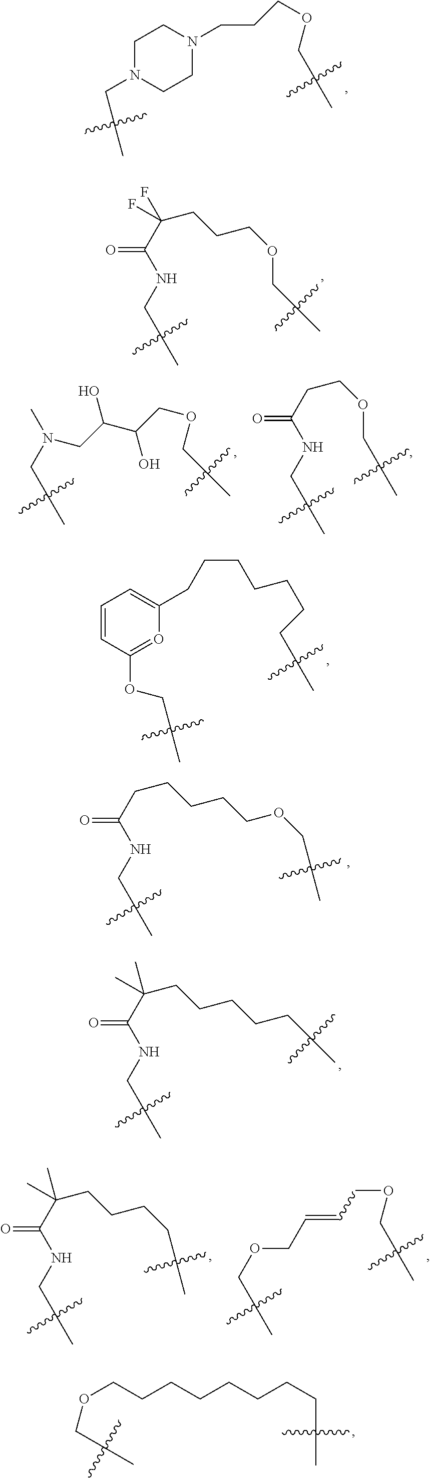

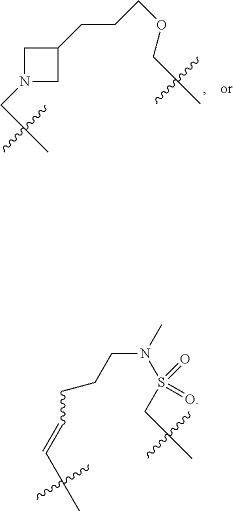 [SMILES: *COCCCC1CN(C*)C1 ; *C=CCCCN(C)S(=O)(=O)C*]

73. The method of any one of claims 1-72, wherein X¹ is N.

74. The method of any one of claims 1-72, wherein X¹ is CR_{c}.

75. The method of claim 74, wherein X¹ is C(CH₃).

76. The method of claim 74, wherein X¹ is CH.

77. The method of any one of claims 1-76, wherein X² is CR_{d}.

78. The method of claim 77, wherein R^{d} is H or optionally substituted C₁-C₆ alkyl.

79. The method of claim 77 or 78, wherein X² is CH.

80. The method of claim 77 or 78 wherein X² is C(CH₃).

81. The method of any one of claims 1-80, wherein X⁵ is CR_{d}.

82. The method of claim 81, wherein X⁵ is CH.

83. The method of any one of claims 1-82, wherein X³ is CR_{f}.

84. The method of claim 83, wherein X⁴ is N.

85. The method of claim 83, wherein X⁴ is CH.

86. The method of any one of claims 1-82, wherein X⁴ is CR_{f}.

87. The method of claim 86, wherein X³ is N.

88. The method of claim 86, wherein X³ is CH.

89. The method of any one of claims 1-88, wherein R_{f} is optionally substituted 5- to 10-membered heteroaryl containing 1, 2, or 3 heteroatoms selected from N, O, and S.

90. The method of claim 89, wherein R_{f} is 6-membered heteroaryl containing 1, 2, or 3 heteroatoms selected from N, O, and S.

91. The method of claim 90, wherein R_{f} is optionally substituted pyrimidinyl.

92. The method of claim 91, wherein R_{f} is:

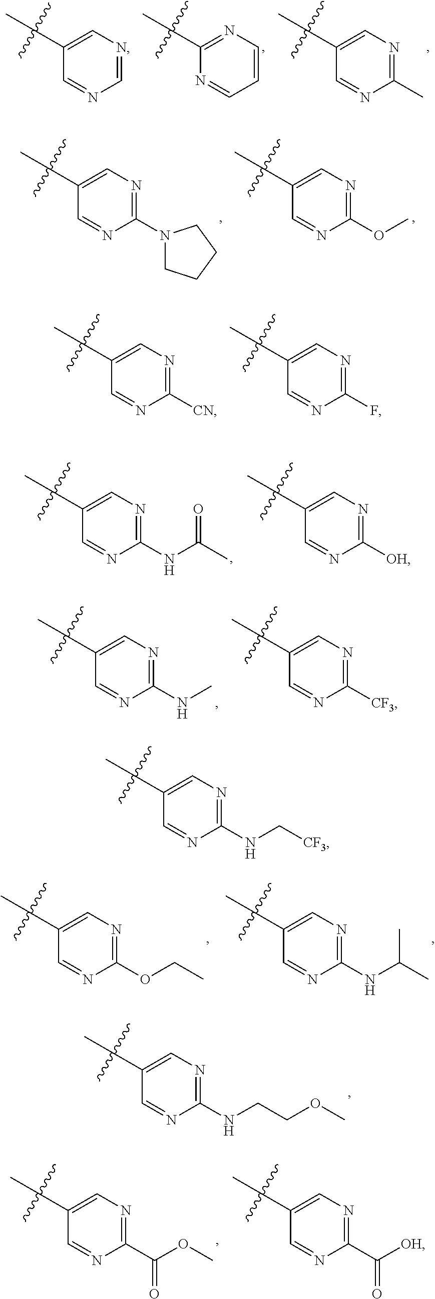

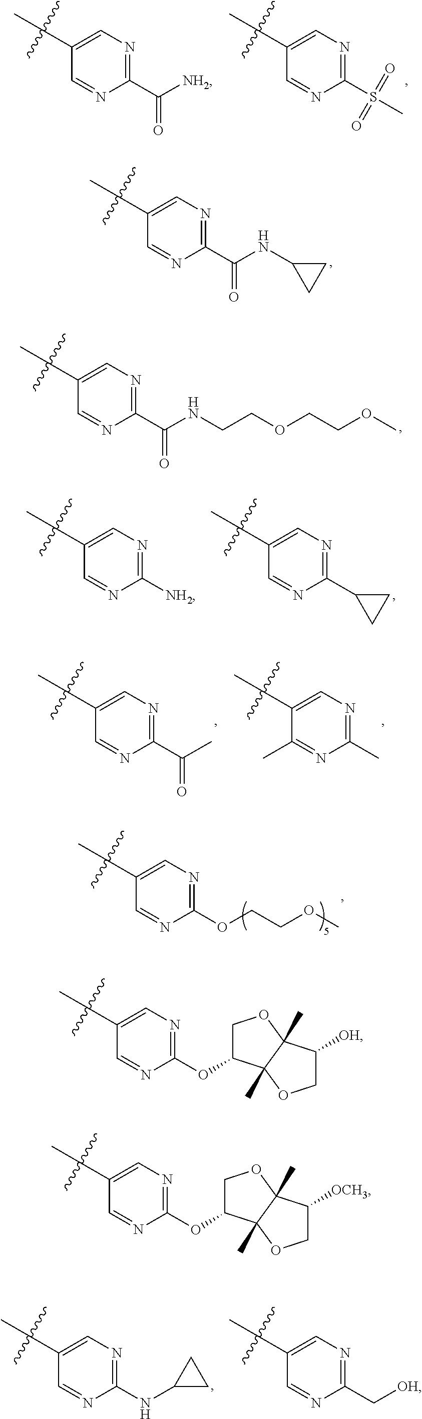

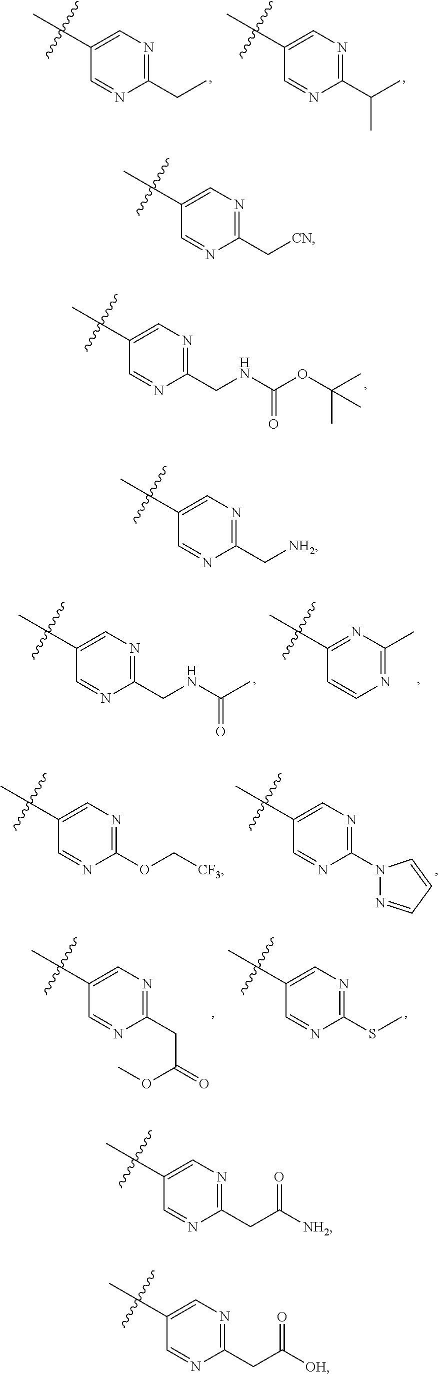

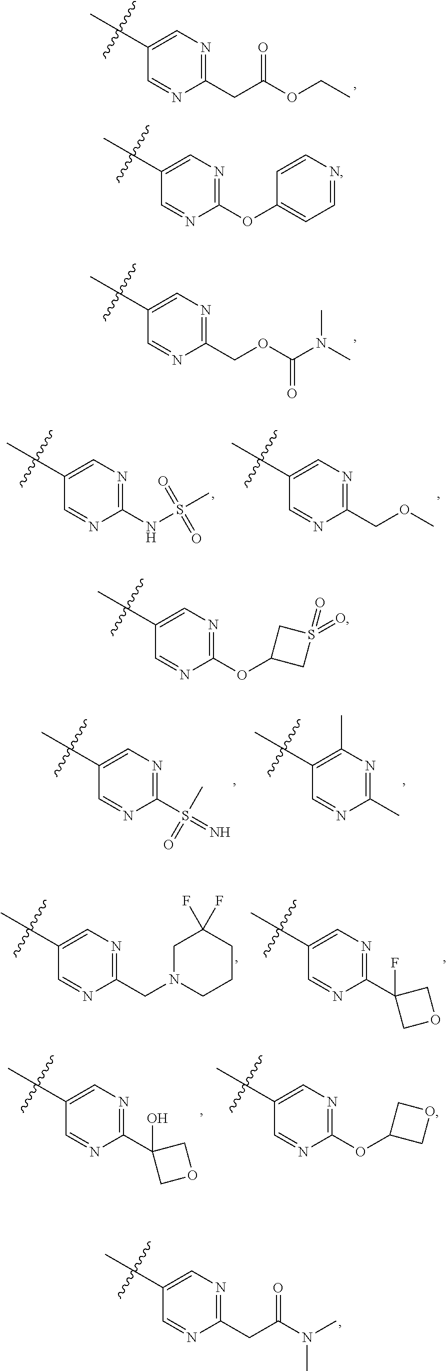

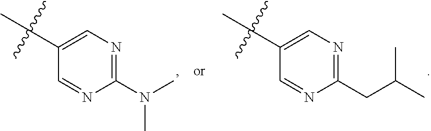

93. The method of claim 92, wherein R_{f} is

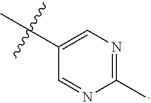 [SMILES: *c1cnc(C)nc1]

94. The method of claim 90, wherein R_{f} is:

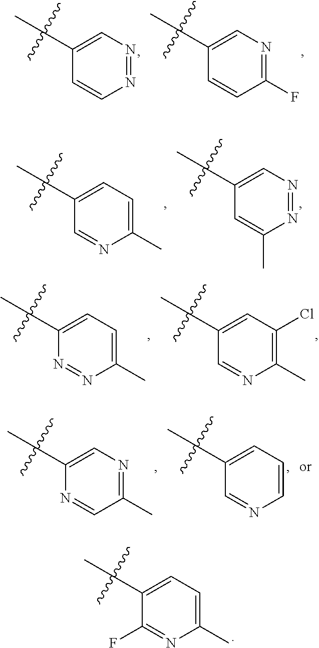

95. The method of claim 89, wherein R_{f} is optionally substituted 8- to 10-membered bicyclic heteroaryl containing 1, 2, or 3 heteroatoms selected from N, O, and S.

96. The method of claim 95, wherein R_{f} is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl.

97. The method of claim 96, wherein R_{f} is:

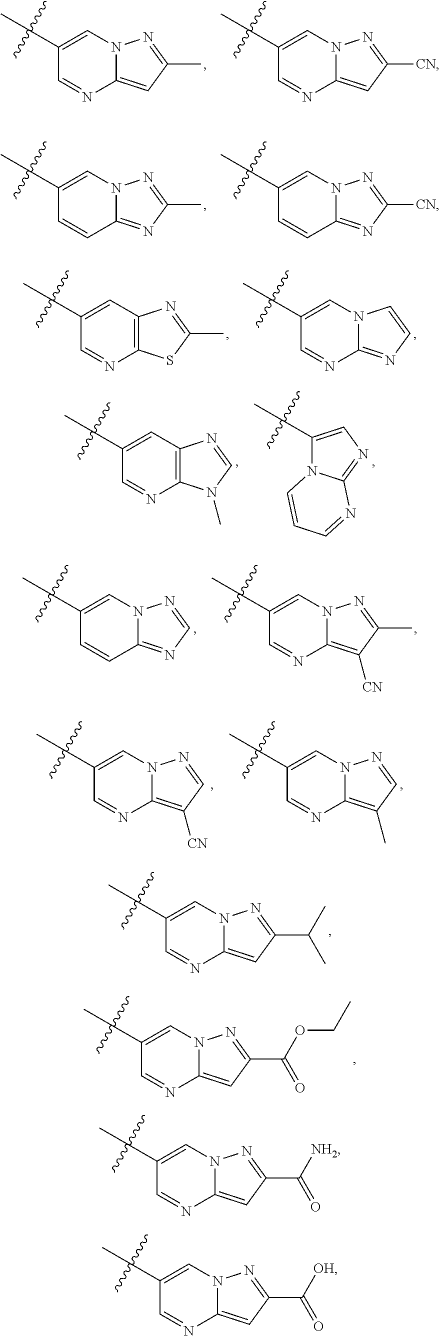

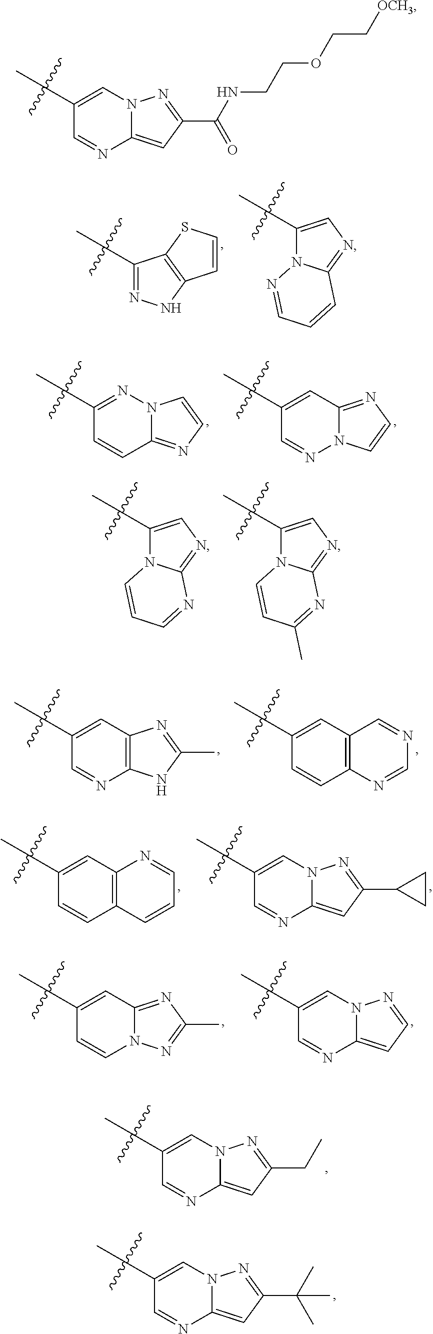

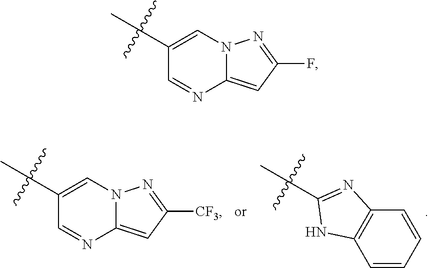 [SMILES: *c1cnc2cc(F)nn2c1 ; *c1cnc2cc(C(F)(F)F)nn2c1 ; *c1nc2ccccc2[nH]1]

98. The method of claim 97, wherein R_{f} is

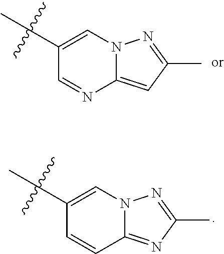 [SMILES: *c1cnc2cc(C)nn2c1 ; *c1ccc2nc(C)nn2c1]

99. The method of any one of claims 1-88, wherein R_{f} is optionally substituted C₆-C₁₄ aryl.

100. The method of claim 99, wherein R_{f} is optionally substituted phenyl.

101. The method of claim 100, wherein R_{f} is:

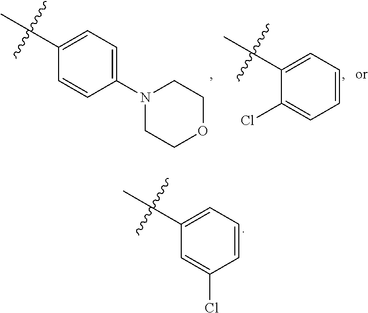 [SMILES: *c1ccc(N2CCOCC2)cc1 ; *c1ccccc1Cl ; *c1cccc(Cl)c1]

102. The method of any one of claims 1-88, wherein R_{f} is optionally substituted 6- to 9-membered unsaturated heterocyclyl containing 1-4 heteroatoms selected from N, O, or S.

103. The method of claim 102, wherein the R_{f} is bonded to the carbon atom to which it is attached through a carbon ring atom contained therein.

104. The method of claim 103, wherein R_{f} is:

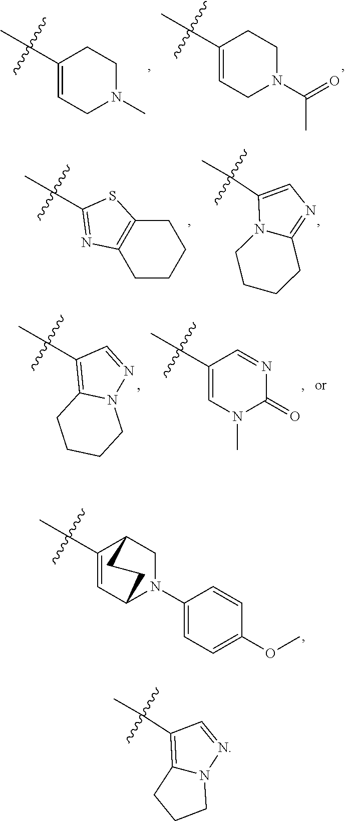

105. The method of claim 102, wherein R_{f} is:

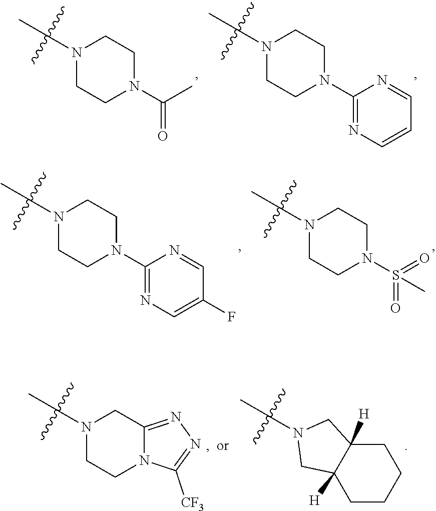

106. The method of claim 89, wherein R_{f} is optionally substituted 5-membered heteroaryl containing 1, 2, or 3 heteroatoms selected from N, O, and S.

107. The method of claim 106, wherein R_{f} is:

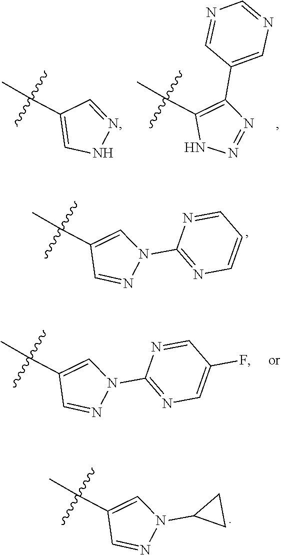 [SMILES: *c1cn[nH]c1 ; *c1[nH]nnc1-c1cncnc1 ; *c1cnn(-c2ncccn2)c1 ; *c1cnn(-c2ncc(F)cn2)c1 ; *c1cnn(C2CC2)c1]

108. The method of any one of claims 1-107, wherein R¹ is —C(O)R_{b}.

109. The method of claim 108, wherein R¹ is

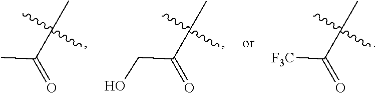

110. The method of claim 109, wherein R¹ is

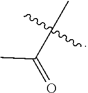 [SMILES: *C(C)=O]

111. The method of any one of claims 1-107, wherein R¹ is —C(O)NRₐRₐ′.

112. The method of claim 111, wherein R¹ is:

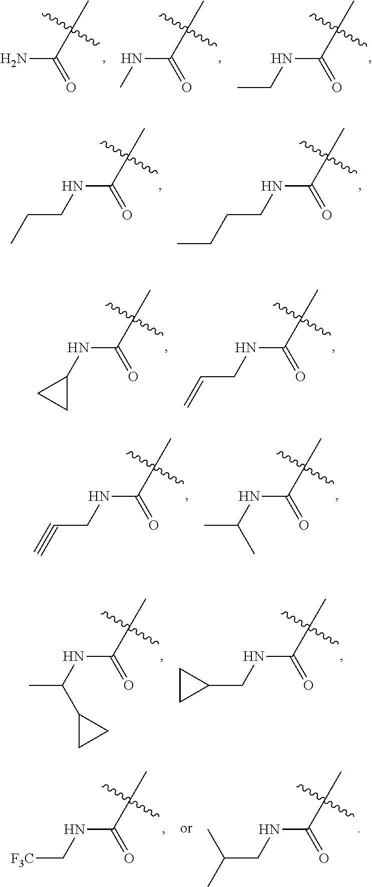

113. The method of claim 112, wherein R¹ is

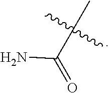 [SMILES: *C(N)=O]

114. The method of any one of claims 1-107, wherein R¹ is —C(O)OR_{b}.

115. The method of claim 114, wherein R¹ is —C(O)OCH₃ or —C(O)OH.

116. The method of any one of claims 1-107, wherein R¹ is optionally substituted C₁-C₆ alkyl.

117. The method of claim 116, wherein R¹ is

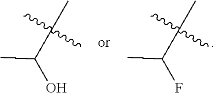

118. The method of any one of claims 1-107, wherein R¹ is

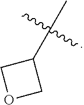 [SMILES: *C1COC1]

119. The method of any one of claims 1-107, wherein R¹ is cyano.

120. The method of any one of claims 1-107, wherein R¹ is halo.

121. The method of any one of claims 1-120, wherein R² is H.

122. The method of any one of claims 1-121, wherein R³ is H.

123. The method of any one of claims 2-9, wherein the compound of formula (III) is: 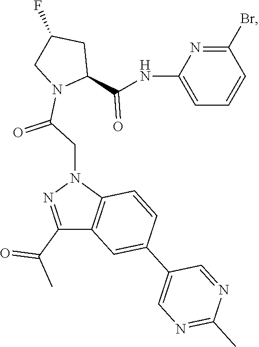 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

124. The method of any one of claims 2-9, wherein the compound of formula (III) is: 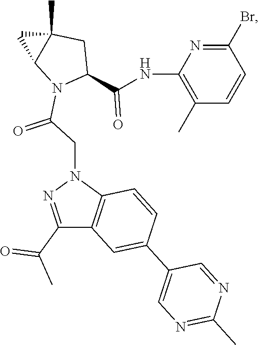 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof.

125. The method of any one of claims 2-9, wherein the compound of formula (III) is: 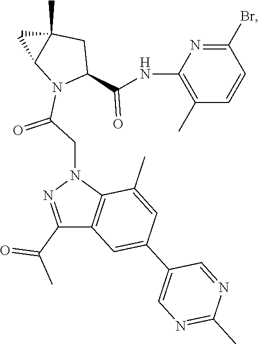 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]
or a pharmaceutically acceptable salt thereof.

126. The method of any one of claims 2-9, wherein the compound of formula (III) is: 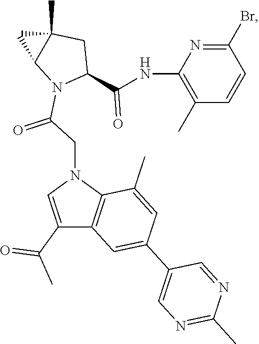 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]
or a pharmaceutically acceptable salt thereof.

127. The method of any one of claims 2-9, wherein the compound of formula (III) is: 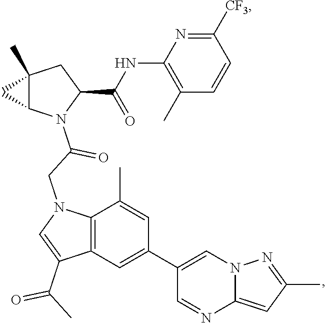 [SMILES: CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12]
or a pharmaceutically acceptable salt thereof.

128. The method of any one of claims 2-9, wherein the compound of formula (III) is: 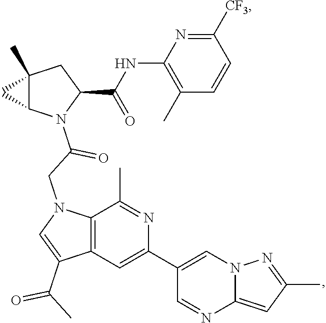 [SMILES: CC(=O)c1cn(CC(=O)N2[C@H](C(=O)Nc3nc(C(F)(F)F)ccc3C)C[C@@]3(C)C[C@@H]23)c2c(C)nc(-c3cnc4cc(C)nn4c3)cc12]
or a pharmaceutically acceptable salt thereof.

129. The method of any one of claims 2-9, wherein the compound of formula (III) is: 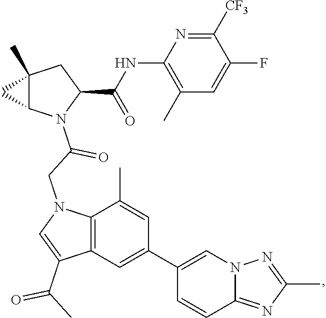 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3ccc4nc(C)nn4c3)cc12]
or a pharmaceutically acceptable salt thereof.

130. The method of any one of claims 2-9, wherein the compound of formula (III) is: 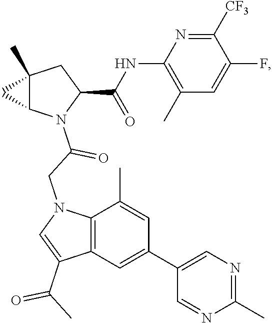 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3cnc(C)nc3)cc12]
or a pharmaceutically acceptable salt thereof.

131. The method of any one of claims 2-9, wherein the compound of formula (III) is: 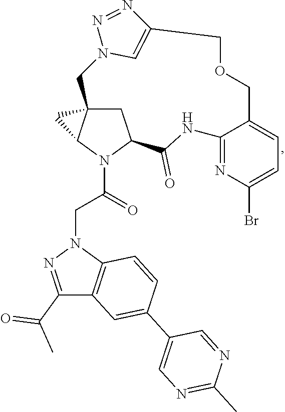 [SMILES: CC(=O)c1nn(CC(=O)N2[C@H]3C[C@]4(C[C@@H]24)Cn2cc(nn2)COCc2ccc(Br)nc2NC3=O)c2ccc(-c3cnc(C)nc3)cc12]
or a pharmaceutically acceptable salt thereof.

132. The method of any one of claims 1-131, wherein the weak base is at least one of potassium carbonate or cesium carbonate.

133. The method of claim 132, wherein the weak base is potassium carbonate.

134. The method of any one of claims 1-133, wherein the water miscible organic solvent is selected from 1,2-propanediol, dimethylformamide, di-isopropylethylamine, or dimethyl sulfoxide.

135. The method of claim 134, wherein the inert, water miscible organic solvent is 1,2-propanediol.
